(19) United States
(12) Patent Application Publication
(10) Pub. No.: US 2023/0242940 A1
(43) Pub. Date: Aug. 3, 2023

(54) METHODS OF MAKING AND USING A VACCINE AGAINST CORONAVIRUS

(71) Applicants: Massachusetts Eye and Ear Infirmary, Boston, MA (US); The Schepens Eye Research Institute, Inc., Boston, MA (US)

(72) Inventors: Luc H. Vandenberghe, Basel (CH); Urja Achal Bhatt, Boston, MA (US); Nerea Zabaleta Lasarte, Boston, MA (US); Wenlong Dai, Boston, MA (US)

(73) Assignees: Massachusetts Eye and Ear Infirmary, Boston, MA (US); The Schepens Eye Research Institute, Inc., Boston, MA (US)

(21) Appl. No.: 17/918,878

(22) PCT Filed: Apr. 13, 2021
(86) PCT No.: PCT/US2021/027153

§ 371 Date: Oct. 13, 2022

Related U.S. Application Data
(60) Provisional application No. 63/033,754, filed on Jun. 2, 2020.
(60) Provisional application No. 63/020,533, filed on May 5, 2020.
(60) Provisional application No. 63/009,319, filed on Apr. 13, 2020.

(51) Int. Cl. C12N 15/86; A61K 39/215; A61P 31/14

(52) U.S. Cl. CPC C12N 15/86; A61K 39/215; A61P 31/14; A61K 2039/54; C12N 2750/14143; C12N 2750/14171

(57) ABSTRACT

Provided herein are vaccines against coronavirus that utilize adeno-associated virus (AAV) for delivery.

### TECHNICAL FIELD

This disclosure generally relates to methods of making and using viral vectors in vaccines against coronavirus.

### BACKGROUND

The spread of SARS-nCoV-2 has reached pandemic proportions, putting at risk healthcare systems. The establishment of population immunity through vaccination is likely the only tool currently available that can stem an epidemic of this proportion without major loss of life. Genetic vaccine strategies have a benefit over traditional vaccines as they can be tested, manufactured, and scaled more rapidly.

Adeno-associated virus (AAV) is a gene transfer platform with an exceptional safety profile in over 25 years and hundreds of interventional clinical trials in gene therapy. This disclosure describes an AAV viral vector that can be used in a vaccine against coronavirus.

### SUMMARY

In one aspect, viral vectors are provided that include an adeno-associated virus (AAV) vector that includes an antigenic region of a coronavirus.

In some embodiments, the AAV vector is naturally occurring primate AAV. In some embodiments, the AAV is an engineered or synthetic AAV. In some embodiments, the AAV vector is selected from AAV1, AAV4, AAV5, AAV6, AAV8, AAV11 and rh32.33. In some embodiments, the AAV vector is AAV11 or Rh32.33.

In some embodiments, the coronavirus is SARS-nCoV-2019. In some embodiments, the antigenic region of a coronavirus comprises one or more SPIKE regions or a portion thereof. In some embodiments, the SPIKE region or a portion thereof comprises an S1 domain or a RBD domain. In some embodiments, the SPIKE region or a portion thereof is stabilized. In some embodiments, the stabilization comprises mutagenesis or codon optimization, cross-linking, or heteromerization or homomerization. In some embodiments, the stabilization comprises removal of a furin cleavage site. In some embodiments, the stabilization comprises the addition of a trimerization C-terminal domain.

In some embodiments, the viral vector is configured for intramuscular delivery. In some embodiments, the viral vector further includes an adjuvant. Representative adjuvants include, without limitation, IL-2, IL-12, IL-18, IFN-gamma, or Niv G, a nucleic acid encoding the adjuvant, Freund’s adjuvant or montanide.

In some embodiments, the viral vector further comprises a nucleic acid sequence encoding kanamycin resistance.

In another aspect, methods of vaccinating a subject against coronavirus are provided. Such methods typically include: providing a viral vector that includes an adeno-associated virus (AAV) vector that includes an antigenic region of a coronavirus; and delivering the viral vector to a subject.

In some embodiments, the subject is a human, a companion animal, an exotic animal, or a livestock animal. In some embodiments, the viral vector is delivered intramuscularly. In some embodiments, the viral vector is delivered intranasally or subcutaneously.

In some embodiments, the viral vector is delivered prior to exposure or infection. In some embodiments, the viral vector is delivered following exposure or infection.

In some embodiments, the subject exhibits a protective immune response. In some embodiments, the protective immune response comprises an increase in Th1 cells. In some embodiments, the protective immune response comprises an increase in Treg cell ratios. In some embodiments, the protective immune response comprises an amelioration of cytokine storms, ARDS and/or myocardial damage severity. In some embodiments, the subject exhibits decreased lymphocyte counts, decreased erythrocyte sedimentation rates following delivery, and/or decreased C-reactive protein levels.

In some embodiments, the methods can further include delivering the viral vector with: one or more antibodies or peptides that block the interaction of the coronavirus with ACE2; one or more antibodies or peptides that promote proteolysis or enzyme deactivation of ACE2; gene editing components (e.g., CRISPR-Cas9, CRISPR-Cas13, ADAR, etc.) to edit ACE2 nucleic acid sequences to reduce or block the interaction of the coronavirus with ACE2; one or more agents that enhance the immunogenicity of the capsid of a virus produced from the viral vector; one or more agents that reduce the expression of the coronavirus (e.g., Remdesivir); one or more agents that promote proteolysis or enzymatic deactivation of the SPIKE protein; one or more agents that degrade or deactivate the TMPRSS2 enzyme of the coronavirus to prevent entry of the virus into the host cell (e.g., Camostat).

In yet another aspect, methods of producing a viral vaccine are provided. Such methods typically include providing a population of adherent or suspension cells; infecting the adherent cells with the viral vector; and culturing the infected cells under conditions in which the virus replicates. In some embodiments, the cells are baculovirus cells. In some embodiments, the culturing step is performed in a bioreactor.

In still yet another aspect, viral vectors are provided that include a sequence having at least 95% sequence identity to SEQ ID NOs: 1, 2, 3, 4, 5, 9, 11, or 13. In some embodiments, the viral vector has at least 99% sequence identity to SEQ ID NOs: 1, 2, 3, 4, 5, 9, 11, or 13. In some embodiments, the viral vector has the sequence shown in SEQ ID NOs: 1, 2, 3, 4, 5, 9, 11, or 13.

In another aspect, viral vectors as shown in Construct 1, Construct 2, Construct 3, Construct 4, Construct 5, Construct 6, Construct 7, Construct 8, Construct 9, or Construct 10 are provided.

In yet another aspect, viral vectors selected from the following are provided: (a) rh32.33 AAV containing the full-length SPIKE protein (AAVrh.32.33.FL-S); (b) rh32.33 AAV containing the S1 domain of the SPIKE protein (AAVrh.32.33.S1) (see, e.g., COVID19-3 (SEQ ID NOs: 13 and 14)); (c) rh32.33 AAV containing the RBD of the SPIKE protein (AAVrh.32.33 RBD); (d) self complementary rh32.33 AAV containing the RBD of the SPIKE protein (scAAVrh32.33.RBD) (see, e.g., AAVCOVID19-2 (SEQ ID NOs: 11 and 12)); (e) rh32.33 AAV containing the full-length SPIKE protein containing at least one set of furin or proline stabilization mutations or combinations thereof (AAVrh.32.33.FL-S stable version 1, 2, 3, etc.) (see, e.g., AAVCOVID19-1 (SEQ ID NOs: 9 and 10)); (f) rh32.33 AAV containing the ectodomain of the SPIKE protein containing at least one set of furin or proline stabilization mutations or combinations thereof (AAVrh.32.33.ectodomain S version 1, 2, 3, etc.); (g) rh32.33 AAV containing the full-length SPIKE protein containing at least one set of furin or proline stabilization mutations or combinations thereof with trimerization modifications (AAVrh.32.33.FL-S Tri stable version 1, 2, 3, etc.); or (h) rh32.33 AAV containing the ectodomain of the SPIKE protein containing at least set of furin or proline one stabilization mutations or combinations thereof with trimerization modifications (AAVrh.32.33.ectodomain S Tri version 1, 2, 3, etc.). It is noted that the rh32.33 AAV in any of the constructs above can be replaced with AAV 11.

In another aspect, a viral vector is provided that includes an amino acid sequence having at least 95% sequence identity to SEQ ID NOs: 5, 10, 12, 14, 22, 24, 26, or 28 (e.g., at least 99% sequence identity to SEQ ID NOs: 5, 10, 12, 14, 22, 24, 26, or 28; the amino acid sequence shown in SEQ ID NOs: 5, 10, 12, 14, 22, 24, 26, or 28).

In still another aspect, a viral vector is provided that includes a nucleic acid sequence having at least 95% sequence identity to SEQ ID NOs: 1, 2, 3, 4, 9, 11, 13, 15, 16, 17, 18, 23, 25, 27, or 29 (e.g., at least 99% sequence identity to SEQ ID NOs: 1, 2, 3, 4, 9, 11, 13, 15, 16, 17, 18, 23, 25, 27, or 29; the nucleic acid sequence shown in SEQ ID NOs: 1, 2, 3, 4, 9, 11, 13, 15, 16, 17, 18, 23, 25, 27, or 29).

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 shows the sequence (SEQ ID NO:1) and a structural schematic of Construct 1, designated “pK.S1-2”.

FIG. 2 shows the sequence (SEQ ID NO:2) and a structural schematic of Construct 2, designated “pK.sc-RBD-2”.

FIG. 3 shows the sequence (SEQ ID NO:3) and a structural schematic of Construct 3, designated “pK.ss-RBD-2”.

FIG. 4 shows the sequence (SEQ ID NO:4) and a structural schematic of Construct 4, designated “pK.FL-5”.

FIGS. 5A-5E are cartoons that show the generalized schematic of Constructs 1-4 and production of Construct 5, respectively.

FIG. 6 shows a schematic of the AAVCOVID19-1 vector.

FIG. 7 shows a schematic of the AAVCOVID19-2 vector.

FIG. 8 shows a schematic of the AAVCOVID19-3 vector.

FIG. 9A is a schematic representation of the recombinant genome of AAVCOVID19-1 (AC1) and AAVCOVID19-3 (AC3) vaccine candidates. SV40: Simian virus 40 promoter. RBD: receptor binding domain. S1: SARS-CoV-2 Spike subunit 1. S2: SARS-CoV-2 Spike subunit 2. CMV: cytomegalovirus promoter. tPA-SP: tissue plasminogen activator signal peptide. WPRE: woodchuck hepatitis virus posttranscriptional regulatory element. bGH: bovine growth hormone. ITR: inverted terminal repeat.

FIG. 9B is a phylogenetic tree of several AAV clades and their percent sequence identity with AAVrh32.33.

FIG. 9C is a schematic showing the percentage of seropositivity of neutralizing antibodies and titer range against AAV2, AAV8 and AAVrh32.33 among 50 donor plasma samples.

FIG. 9D is a graph that shows the productivity of several AC1 and AC3 (vector genome copies produced per producer cell or Gc/cell) compared to various AAV serotypes carrying a CMV-EGFP-WPRE transgene in small-scale production and purification. Data are represented as mean ± SD. One-way ANOVA and Tukey’s tests were used to compare groups between them. * p<0.05, ** p<0.01.

FIG. 9E is a graph that shows AAV-ID analysis of capsid identity and stability of AC1 and AC3 compared to AAVrh32.33 and other serotypes.

FIG. 9F is a photograph that shows the detection of SARS-CoV-2 Spike antigens by Western blot in HEK293 cells transfected with 1 µg of ITR-containing pAC1 or pAC3 plasmids or Huh7 cells transduced with 5 × 10e5 gc/cell of AC1 and AC3 72 h after treatment. Recombinant S ectodomain (S ecto, lane 1) and S1 subunit (S1, His-tagged, lane 2) were used as positive control and size reference.

FIGS. 10A-10B are graphs that show the results of monthly monitoring of SARS-CoV-2 RBD-binding IgG titers in 6-10 week-old BALB/c (FIG. 10A) and C57BL/6 (FIG. 10B) mice injected IM with two doses (10e10 gc and 10e11 gc) of AC1 or AC3, n=20 (10 females and 10 males). Mean geometric titers (MGT) shown above each group. * p<0.05, ** p<0.01, *** p<0.001, **** p<0.0001.

FIGS. 10C-10D are graphs that show pseudovirus neutralizing titers of a subset of BALB/c (FIG. 10C) and C57BL/6 (FIG. 10D) animals (6 females and 6 males per group) from the studies described in FIG. 10A and FIG. 10B. The GMT are shown above each group. * p<0.05, ** p<0.01, *** p<0.001, **** p<0.0001.

FIGS. 10E-10F are graphs that show the correlation of pseudovirus neutralizing titers and RBD-binding IgG titers in BALB/c (FIG. 10E) and C57BL/6 (FIG. 10F).

FIG. 10G is a graph that shows live SARS-CoV-2 neutralizing titers measured on a PRNT assay on week 4 samples harvested from BALB/c animals (n≥8, both genders). The GMT is shown above each group. * p<0.05, ** p<0.01, *** p<0.001, **** p<0.0001.

FIG. 10H is a graph that shows the correlation of SARS-CoV-2 neutralizing and pseudovirus neutralizing titers.

FIG. 10I is a graph that shows the titer of binding antibodies against SARS-CoV-2 RBD (SARS2 RBD), SARS-CoV-2 Spike ectodomain (SARS2 Ecto) and SARS-CoV RBD (SARS RBD) in female BALB/c sera 28 days after AC1 or AC3 injection.

FIG. 10J is a graph that shows RBD-binding antibody titers in BALB/c male animals (n=5) vaccinated with 10e11 gc of AC1 or AAV1-S (same genomic sequence packaged in different capsids), which were naïve (0 mg IVIG) or passively pre-immunized with 15 mg of human IVIG 24 h and 2 h prior to the vaccination. Ctr: unvaccinated control.

FIG. 11A is a graphical representation that shows several RBD-binding antibody isotype titers (IgG, IgG1, IgG2a, IgG2b, IgG3, IgA and IgM) measured weekly in 6-10 week-old BALB/c (n=10, 5 females and 5 males) treated IM with two doses of AC1 and AC3.

FIG. 11B is a graph that shows the ratio of RBD-binding IgG2a and IgG1 antibody titers in serum samples harvested 28 days after vaccination of BALB/c mice as described in FIG. 11A. The Geometric Mean Titer (GMT) is shown above each group.

FIGS. 11C and 11F are graphs that show the cytokine concentration (pg/mL) in supernatants harvested from splenocytes stimulated for 48 h with peptides spanning SARS-CoV-2 Spike protein. Splenocytes were extracted from BALB/c (FIG. 11C) and C57BL/6 (FIG. 11F) animals 4 and 6 weeks, respectively, after vaccination with 10e11 gc of AC1 or AC3.

FIGS. 11D-11E shows spot forming units (SFU) detected by IFN-gamma (FIG. 11D) or IL-4 (FIG. 11E) ELISpot in splenocytes extracted from BALB/c animals 4 weeks after vaccination with 10e11 gc of AC1 or AC3 and stimulated with peptides spanning SARS-CoV-2 Spike protein for 48 h.

FIGS. 11G-11H shows spot forming units (SFU) detected by IFN-gamma (FIG. 11G) or IL-4 (FIG. 11H) ELISpot in splenocytes extracted from C57BL/6 animals 6 weeks after vaccination with 10e10 gc of AC1 or AC3 and stimulated with peptides spanning SARS-CoV-2 Spike protein for 48 h.

FIG. 12A is a graph that shows the RBD-binding antibody titers measured on weeks 2, 4 and 6 in 18 week-old C57BL/6 animals (n ≥ 9, both genders) vaccinated with two doses (10e10 gc and 10e11 gc) of AC1 and AC3 intramuscularly. Mean geometric titers (MGT) shown above each group.

FIG. 12B is a graph that shows the pseudovirus neutralizing titers on week 4 in animals described in FIG. 12A. The Geometric Mean Titer (GMT) is shown above each group.

FIG. 12C is a graph that shows the RBD-binding antibody titers measured on weeks 4, 7 and 13 in 2 year-old C57BL/6 animals (n ≥ 7, both genders) vaccinated with two doses (10e10 gc and 10e11 gc) of AC1 and AC3 intramuscularly. GMT is shown above each group.

FIG. 12D is a graph showing the pseudovirus neutralizing titers on weeks 7 and 13 in animals described in FIG. 12C. GMT is shown above each group.

FIG. 12E is a graph that shows seroconversion rates in RBD-binding antibodies 4 weeks after vaccination of C57BL/6 mice at different ages.

FIG. 12F is a graph that shows RBD-binding antibody titers measured on weeks 2, 3 and 4 in 12-week-old lean and obese C57BL/6 animals (n=10 males) vaccinated with two doses (10e10 gc and 10e11 gc) of AC1 and AC3 intramuscularly. * p<0.05, ** p<0.01, *** p<0.001, **** p<0.0001.

FIG. 12G is a graph that shows pseudovirus neutralizing titers on week 4 in animals described in FIG. 4F. * p<0.05, ** p<0.01, *** p<0.001, **** p<0.0001.

FIG. 13A is a graph showing the pseudovirus neutralizing antibody titers (international units (IU)/mL in the right y axis and reciprocal serum dilution in the left y axis) 36-week follow up in Rhesus macaques (n=2, 1 female and 1 male) treated IM with 10e12 gc of AC1 or AC3. The dotted line indicates the lower detection limit of the assay.

FIG. 13B is a graph showing the pseudovirus neutralizing antibody titers (IU/mL) in 60 convalescent human plasma samples of patients with different disease severity. The Geometric Mean Titer (GMT) is shown for each cohort of convalescent plasma. The dotted line indicates the lower detection limit of the assay.

FIG. 13C is a graph showing the correlation between pseudovirus neutralization (IU/mL) and live SARS-CoV-2 neutralization titers. Pearson’s correlation coefficient was calculated to assess correlation.

FIG. 13D is a graph showing the correlation between pseudovirus neutralization (IU/mL) and VSV pseudovirus neutralization titers measured at the reference lab Nexelis (Laval, Canada). Pearson’s correlation coefficient was calculated to assess correlation.

FIG. 13E shows the SARS-CoV-2 RBD-binding IgG titers 36-week follow up in Rhesus macaques (n=2, 1 female and 1 male) treated IM with 10e12 gc of AC1 or AC3. The dotted line indicates the lower detection limit of the assay.

FIG. 13F is a graph showing the frequency of RBD-binding B cells with a memory phenotype (CD27+ or CD27-IgD-) in the peripheral blood B cell compartment as measured by flow cytometry

FIG. 13G is a graph showing the frequency of isotype-switched (IgD-IgM-) phenotype within RBD-binding memory B cells as measured by flow cytometry.

FIG. 13H is a graph showing the RBD-binding IgG and IgA and pseudovirus neutralizing titers in bronchoalveolar lavage (BAL) samples harvested on week 20 after vaccination, in comparison with IgG and neutralizing titers detected in serum at the same timepoint. Dotted lines indicate the lower detection limit for each measurement.

FIG. 13I is a graph showing quantification of IFN-gamma spot forming units (SFU) by ELISPOT in PBMC samples collected at different timepoints in Rhesus macaques (n=2/vector) treated with AC1 or AC3 and stimulated with peptides specific for each transgene.

FIGS. 14A-14B are graphs that show the quantification of spot forming units (SFU) by ELISpot in PBMC samples collected at different time points in rhesus macaques (n = 2/vector) treated with AC1 (FIG. 14A) or AC3 (FIG. 14B) and stimulated with peptides specific for each transgene.

FIGS. 14C-14D are dot plots summarizing the background subtracted frequency of CD107a+ IFN-gamma⁺ or TNFalpha⁺ IFN-gamma⁺ cells responding to AC1/AC3 and AAVrh32.33 peptide pools at baseline and at different time points after vaccination. The dotted line indicates the cutoff for positive responses.

FIG. 14E shows flow cytometry plots from AC3 female indicating the frequency of Perforin, Granzyme B, Tbet, TNFalpha, IL2 and KI67-positive cells within CD107⁺ IFN-gamma⁺ memory CD8+ T cells responding to AC1/AC3 shared peptide pool B at day 42 and 98 post vaccination. In the flow plots, total CD107⁺ IFN-gamma⁺ cells were depicted as light dots overlayed on total memory CD8⁺ T cells shown as dark dots.

FIG. 14F show dot plots summarizing the frequency of background subtracted TNFalpha⁺ IL2⁺ cells responding to AC1/AC3 and AAVrh32.33 peptide pools at baseline and at different time points after vaccination (n = 4). The dotted line indicates the cutoff for positive responses.

FIG. 15A is a graph that shows the percentage of titer relative to the -80° C. stored control for AC1 and AC3 aliquots stored at 4° C. or room temperature (RT) for 1, 3, 7 or 28 days.

FIG. 15B is a graph that shows the measurement of RBD-binding IgG titers in BALB/c female animals vaccinated with AC1 aliquots kept at several temperatures for 1, 3, 7 or 28 days. Animals received 5 × 10e10 gc IM and antibodies were measured 24 days post-vaccination.

FIG. 16A shows coRBD mRNA expression relative to human 18S rRNA in HEK293 cells transfected with 1 µg of the ITR-containing pAC1 or pAC3 plasmids or transduced with 1×10e5 or 5×10e5 gc/cell of AC1 or AC3 24 h after treatment. Ctr: untreated cells.

FIG. 16B shows unedited Western blot image from FIG. 9G. Red rectangle indicate the part of the gel represented in FIG. 9G.

FIG. 17A shows RBD-binding IgG antibody titers measured weekly during the first month after vaccination in BALB/c (n=5 females and 5 males) treated IM with two doses of AC1 and AC3. Data are represented as mean ± SD. Groups were compared by one-way ANOVA and Tukey’s post-test. * p<0.05, ** p<0.01.

FIG. 17B shows the seroconversion rates of RBD-binding titers represented in FIG. 17A.

FIGS. 18A-18B shows spot forming units (SFU) detected by IFN-gamma (FIG. 18D) or IL-4 (FIG. 18E) ELISpot in splenocytes extracted from BALB/c animals 4 weeks after vaccination with 10e11 gc of AC1 or AC3 and stimulated with 2 µg/ml concanavalin A (positive control) for 48 h.

FIGS. 18C-18D show spot forming units (SFU) detected by IFN-gamma (FIG. 18A) or IL-4 (FIG. 18B) ELISpot in splenocytes extracted from C57BL/6 animals 6 weeks after vaccination with 10e10 gc of AC1 or AC3 and stimulated with 2 µg/ml concanavalin A (positive control) for 48 h.

FIG. 19 shows serum chemistry and complete blood counts in NHP. Measurement of total protein (mg/dL), albumin (g/dL), globulin (g/dL), albumin/globulin ratio (A/G ratio), aspartate aminotransferase (AST or SGOT, measured in IU/L), alanine aminotransferase (ALT or SGPT, measured in IU/L), alkaline phosphatase (ALP, measured in IU/L), gamma-glutamyltransferase (GGT, measured in IU/L), total bilirubin (mg/dL), blood urea (BUN, measured in mg/dL), creatinine (mg/dL), BUN/creatinine ratio, phosphorus (mg/dL), glucose (mg/dL), calcium (mg/dL), magnesium (mEq/dL), sodium (mEq/dL), potassium (mEq/dL), sodium to potassium ratio (Na/K ratio), chloride (mEq/dL), cholesterol (mg/dL), triglycerides (mg/dL), amylase (IU/L), creatine phosphokinase (CPK, measured in IU/L) in serum samples of NHP treated with 10e12 gc of AC1 or AC3 7 days before injection, on the day of injection and 1, 7 and 14 days post-treatment. Hematological analysis of white blood cells (WBC, x10e3 cells/µL), red blood cells (RBC, x10e6 cells/µL), hemoglobin (HB, g/dL), hematocrit (HCT, %), mean corpuscular volume (MCV, fL), mean corpuscular hemoglobin (MCH, pg), mean corpuscular hemoglobin concentration (MCHC, g/dL), platelets (x10e3 cells/µL), absolute neutrophils (cells/µL), absolute lymphocytes (cells/µL), absolute monocytes (cells/µL), absolute eosinophils (cells/µL), prothrombin (seconds), activated partial thromboplastin time (APTT, measured in seconds), fibrinogen (mg/dL) and D-dimers (ng/mL) in EDTA-blood samples of NHP treated with 10e12 gc of AC1 or AC3 7 days before injection, on the day of injection and 1, 7 and 14 days post-treatment.

FIG. 20 shows serum cytokine response to AC1 and AC3 in NHP. Concentration (pg/mL) of IL10, IL2, IL12/23, IL1Rα, IL13, granulocyte-macrophage colony-stimulating factor (GM-CSF, MCP1), IL15, vascular endothelial growth factor (VEGF), IL17alpha, IL18, transforming growth factor alpha (TGFalpha) and IL8 in serum samples of NHP treated with 10e12 gc of AC1 or AC3 7 days before injection, on the day of injection and 1, 7, 14, 21 and 28 days post-treatment.

FIG. 21A is a schematic representation of portion of the Spike protein represented in each peptide pool used for NHP PBMC stimulation.

FIG. 21B shows flow cytometry scatter plots from AC3 female animal showing the frequency of CD107⁺ IFN-gamma⁺ cells within blood Memory CD8⁺ T cells at baseline and at weeks 6 and 14 post-vaccination. The numbers indicate the frequency within the parent population.

FIG. 21C shows cytometry scatter plots from AC3 female animal showing the frequency of TNFalpha⁺ IL2⁺ cells within blood Memory CD4⁺ T cells at baseline and at weeks 6 and 14 post-vaccination. The numbers indicate the frequency within the parent population.

FIG. 22A shows neutralizing antibody titers against the injected vector (AAVrh32.33) and cross-reactive neutralizing against other serotypes (AAV1, AAV2, AAV5, AAV8, AAV9).

FIG. 22B shows quantification of spot forming units (SFU) by ELISpot in PBMC samples collected at different timepoints in animals treated with AC1 or AC3 and stimulated with peptides spanning AAVrh32.33 capsid sequence.

FIG. 23A shows quantification of vector genome copies (DNA) in the right gastrocnemius (right gastroc) or injection site, left gastrocnemius (left gastroc) or contralateral muscle, liver and spleen on weeks 1, 4 and 8 after the administration of 10e11 gc of AC1 or AC3 in C57BL/6 animals (n=6, 3/gender). Horizontal dotted lines indicate background levels for right and left gastrocnemius muscles. Liver and spleen had not detectable background. Data are represented as mean ± SD.

FIG. 23B shows quantification of transgene expression in the right gastrocnemius muscle on weeks 1, 4 and 8 in C57BL/6 animals (n=3/gender) injected with 10e11 gc of AC1 or AC3. Data are represented as mean ± SD.

FIG. 23C shows a table of transgene expression values in experiment describe in FIG. 23A. ND: not detected.

FIG. 23D shows biodistribution of AC3 in several organs 8 weeks after vaccination of BALB/c females treated with 10e11 gc. Data are represented as mean ± SD.

FIG. 24A is a graph showing SARS-CoV-2 Spike ectodomain-binding IgG (measured in absorbance units (AU)/mL) elicited by AC1 in Cynomolgus macaques (n=6) during 9 weeks after vaccination. The dotted line indicates the lower detection limit of the assay. Data are represented as geometric mean titer (GMT) ± geometric SD. Mann Whitney test was used to compare vaccinated and control groups.

FIG. 24B is a graph showing the measurement of antibodies that inhibit binding of Spike to ACE2 in an in vitro binding inhibition assay. Data are represented as geometric mean titer (GMT) ± geometric SD. Mann Whitney test was used to compare vaccinated and control groups.

FIG. 24C is a graph showing quantification of IFN-gamma spot forming units (SFU) by ELISpot in PBMC samples collected at different time points in unvaccinated (Control) and AC1-treated cynomolgus macaques (n=6) and stimulated with peptides specific for each transgene. Data are represented as median ± interquartile range. Mann Whitney test was used to compare vaccinated and control groups.

FIGS. 24D-24F are graphs showing SARS-CoV-2 viral RNA copies in nasopharyngeal (24D) and tracheal swab (24E) at several time points after 10e5 pfu SARS-CoV-2 challenge and in bronchoalveolar lavage (BAL) fluid (24F) at day 3 after challenge. Data are represented as median ± interquartile range. Mann Whitney test was used to compare vaccinated and control groups.

FIGS. 24G-24I are graphs showing SARS-CoV-2 subgenomic RNA quantification (copies/mL) in copies in nasopharyngeal (24G) and tracheal swab (24H) at several timepoints after 10e5 pfu SARS-CoV-2 challenge and in bronchoalveolar lavage (BAL) fluid (24I) at day 3 after challenge. Data are represented as median ± interquartile range. Mann Whitney test was used to compare vaccinated and control groups.

FIG. 24J is a graph showing CT score in lungs of control and vaccinated animals before and after challenge. Scores were calculated based on lesion type (scored from 0 to 3) and lesion volume (scored from 0 to 4) for each lobe. Data are represented as median ± interquartile range. Mann Whitney test was used to compare vaccinated and control groups.

FIG. 24K is a graph showing measurement of lung lymph node (LN) activation measured by PET as mean standardized uptake values (SUV mean) before and after challenge. Data are represented as median ± interquartile range. Mann Whitney test was used to compare vaccinated and control groups.

FIG. 25A is a graph showing the titer (gc/mL) of AC1 aliquots (n=5) stored at -80° C., 4° C. and 25° C. for 4 weeks. Data are represented as mean ± SD. One-way ANOVA was used to compare the three groups.

FIG. 25B is a graph showing RBD-binding antibody titers in C57BL/6 animals 21 days after vaccination with 10e1 1gc of AC1 aliquots (n=5) stored at -80° C., 4° C. and 25° C. for 4 weeks. Data are represented as mean ± SD. One-way ANOVA was used to compare the three groups.

FIG. 25C is a graph showing AAV-ID analysis of capsid identity in AC1 aliquots (n=5) stored at -80° C., 4° C. and 25° C. for 4 weeks. Data are represented as mean ± SD.

FIG. 26 is a graph showing the monitoring of SARS-CoV-2 RBD-binding IgG titers in 7-8 week-old C57BL/6 mice injected intramuscularly (IM), subcutaneously (SC) or intranasally (IN) with 5 × 10e10 gc AC3. n=3.

FIGS. 27A-27B are graphs showing MS21_Balb/c mice study in females comparing AAV11 and Rh32.33 vectors with antigen wild type stabilized Spike.

FIG. 28 is a graph showing MS21_Balb/c mice study in females comparing AAV 11 and Rh32.33 vectors with antigen wild type stabilized Spike.

FIGS. 29A-29B are graphs showing MS24_C57BL/6 mice study in both genders comparing AAV11 and Rh32.33 vectors with antigen wild type stabilized Spike.

FIGS. 30A-30B are graphs showing MS24-Elispot on low doses.

FIG. 31 shows additional AAVCOVID constructs.

TABLE OF SEQUENCES

| SEQ ID NO: | Description |
|---|---|
| 1 | Construct 1, designated “pK.S1-2” |
| 2 | Construct 2, designated “pK.sc-RBD-2” |
| 3 | Construct 3, designated “pK.ss-RBD-2” |
| 4 | Construct 4, designated “pK.FL-5” |
| 5 | amino acid sequence of Construct 4 |
| 6 | GCN4 trimerization domain |
| 7 | T4 fibritin trimerization domain |
| 8 | modified isoleucine zipper that has four glycosylation motif |
| 9 | nt sequence of AAVCOVID19-1 |
| 10 | aa sequence of AAVCOVID19-1 |
| 11 | nt sequence of AAVCOVID19-2 |
| 12 | aa sequence of AAVCOVID19-2 |
| 13 | nt sequence of AAVCOVID19-3 |
| 14 | aa sequence of AAVCOVID19-3 |
| 15 | pACE-B.1.351 |
| 16 | pACE-P.1 |
| 17 | pACM-B.1.351 |
| 18 | pACM-P.1 |
| 19 | Primer |
| 20 | Primer |
| 21 | Primer |
| 22 | SARS-nCOV-2 S full length WT: Construct 4 |
| 23 | AAVCOVID19-1 plasmid DNA sequence |
| 24 | AAVCOVID19-1 Translation Sequence |
| 25 | AAVCOVID19-2 plasmid DNA Sequence |
| 26 | AAVCOVID19-2 Translation Sequence |
| 27 | AAVCOVID19-3 plasmid Sequence |
| 28 | AAVCOVID19-3 Translation Sequence |
| 29 | AAV11 sequence in pKan backbone |

### DETAILED DESCRIPTION

AAV is a recombinant viral vector technology based on a 25 nm ssDNA dependovirus of the family of Parvoviridae. Decades of development have led to the FDA approval of two AAV-based drugs (voretigene neparvovec (LUXTERNA®) and onasemnogene abeparvovec (ZOLGENSMA®) for the treatment of an inherited form of blindness and spinal muscular atrophy type 1, respectively). Its favorable safety profile was established following thousands of clinical trial subjects and hundreds of clinical studies over the past 25 years. Moreover, the dose for a genetic immunization is generally orders of magnitude lower than in gene therapy, resulting in an extremely low safety risk for the AAV platform in line with that of a vaccine for broad use in the population.

Although any serotype of AAV can be used in the viral vector described herein (e.g., AAV1, AAV4, AAV5, AAV6, AAV8), AAV11, an AAV isolated from cynomolgus monkeys, or AAVrh32.33, an engineered hybrid of two AAV capsid PCR isolates from rhesus macaque, are particularly useful. See, for example, U.S. Pat. No. 10,301,648 and GenBank Accession No. ACB55318, as well as Mori et al. (2004, Virology, 330:375-83) and GenBank Accession No. AAT46339.1. AAV11 and AAVrh32.33 are highly divergent structurally and serologically from other primate AAVs, with the closest homology to AAV4 (having 65% sequence identity to AAV11 and 81% sequence identity to AAVrh32.33). AAV11 and AAVrh32.33 productively transduce myofibers following intramuscular (IM) injection in mice. Yet, unlike other AAVs, transduction with AAV11 or AAVrh32.33 leads to local inflammation and ultimately a loss of transduced fibers. This process is driven by a CD4, CD40L, and CD28 T-cell mediated killing that is specific to the transgene antigen. AAV11 and AAVrh32.33 gain cell entry from the acidifying endosome via a common entry factor on the host cell in mice and human, referred to as GPR108, yet, unlike other primate AAVs, does not depend on the ubiquitous receptor, AAVR, on the cell surface. Importantly, AAV11 and rh32.33 further differentiate themselves from other AAVs by their low level of pre-existing immunity in human populations based on a screen of a thousand serum samples from four different continents. The immunizing effect via IM is unaffected by high dose systemic IVIG (pooled human serum) in mice and in NHP, and AAV via IM injection is less subject to neutralization than mice. AAV11 and AAVrh32.33 are attractive vaccine candidates as they trigger a multifaceted pro-inflammatory activation that stimulates a strong antibody response that also engages Th1 pathways and promotes Treg homeostasis, generates viral titre high yields, which are essential for large-scale vaccine production, and has a very low seroprevalence in humans.

AAV viral vectors as described herein can contain a nucleic acid molecule that encodes an antigenic polypeptide. AAV viral vectors are commercially available or can be produced by recombinant technology. A viral vector can have one or more elements for expression operably linked to the nucleic acid molecule that encodes an antigenic polypeptide, and further can include sequences such as those encoding a selectable marker (e.g., an antibiotic resistance gene), and/or those that can be used in purification of a polypeptide (e.g., 6xHis tag). Elements for expression include nucleic acid sequences that direct and regulate expression of nucleic acid coding sequences. One example of an expression element is a promoter sequence. Expression elements also can include one or more of introns, enhancer sequences, response elements, or inducible elements that modulate expression of a nucleic acid molecule. Expression elements can be of bacterial, yeast, insect, mammalian, or viral origin and vectors can contain a combination of expression elements from different origins. As used herein, operably linked means that elements for expression are positioned in a vector relative to a coding sequence in such a way as to direct or regulate expression of the coding sequence.

An AAV viral vector can include the necessary components for assembling and packaging (e.g., rep sequences, cap sequences, inverted terminal repeat (ITR) sequences), or such components can be provided on a separate vector. The components of a virus particle can be introduced, transiently or stably, into a packaging host cell such that virus particles are produced. Such virus particles can be purified using routine methods. As used herein, “purified” virus particles refer to virus particles that are removed from components in the mixture in which they were made such as, but not limited to, viral components (e.g., rep sequences, cap sequences), packaging host cells, and partially- or incompletely-assembled virus particles.

Once assembled, virus particles can be screened, e.g., for the ability to replicate; receptor binding ability; and/or seroprevalence in a population (e.g., a human population). Determining whether a virus particle can replicate is routine in the art and typically includes infecting a host cell with an amount of virus particles and determining if the virus particles increase in number over time, and determining whether a virus particle binds to its receptor is routine in the art, and such methods can be performed in vitro or in vivo. Determining the seroprevalence of a virus particle is routinely performed in the art and typically includes using an immunoassay to determine the prevalence of one or more antibodies in samples (e.g., blood samples) from a particular population of individuals. Seroprevalence is understood in the art to refer to the proportion of subjects in a population that is seropositive (i.e., has been exposed to a particular pathogen or immunogen), and is calculated as the number of subjects in a population who produce an antibody against a particular pathogen or immunogen divided by the total number of individuals in the population examined. Similarly, several methods to determine the extent of neutralizing antibodies in a serum sample are available. For example, a neutralizing antibody assay measures the titer at which an experimental sample contains an antibody concentration that neutralizes infection by 50% or more as compared to a control sample without antibody. See, also, Fisher et al. (1997, Nature Med., 3:306-12); and Manning et al. (1998, Human Gene Ther., 9:477-85).

As used herein, Coronavirus refers to SARS-CoV-2 and variants of SARS-CoV-2. The sequence of SARS-nCoV-2 can be found, for example, at GenBank Accession No. MN908947.3, and a number of SARS-CoV-2 variants have been identified (e.g., South African, UK, and Brazil variants; see, e.g., van Oosterhout et al., 2021, Virulence, 12:507-8). Antigenic portions of coronavirus are known and include, for example the extracellular ectodomain portion, which includes the glycoprotein SPIKE region or a portion thereof (e.g., the globular S1 subunit or the receptor binding domain (RBD)). In some instances, more than one (e.g., a plurality of) antigenic sequences can be used in an AAV viral vector.

An AAV viral vector carrying an antigenic portion of a coronavirus can be used as a vaccine to immunize subjects against coronavirus infection, i.e., to elicit a protective immune response that reduces the risk of the subjects developing the infection, or reduces the risk of the subject developing a severe infection. Such a vaccine can be prepared as a vaccine composition, e.g., suspended in a physiologically compatible carrier and administered to a subject (e.g., a human, a companion animal, an exotic animal, and livestock). Suitable carriers include saline, which may be formulated with a variety of buffering solutions (e.g., phosphate buffered saline), lactose, sucrose, calcium phosphate, gelatin, dextran, agar, pectin, and water.

The vaccine composition can include one or more adjuvants. Some adjuvants contain a substance designed to protect the antigen from rapid catabolism, such as aluminum hydroxide or mineral oil, and a specific or nonspecific stimulator of immune responses, such as lipid A, or Bortadellapertussis. Suitable adjuvants are commercially available and include, for example, Freund’s Incomplete Adjuvant and Freund’s Complete Adjuvant (Difco Laboratories) and Merck Adjuvant 65 (Merck and Company, Inc., Rahway, N.J.). Other suitable adjuvants include alum, biodegradable microspheres, monophosphoryl lipid A, quil A, SBAS1c, SBAS2 (Ling et al., 1997, Vaccine 15:1562-1567), SBAS7, Al(OH)3 and CpG oligonucleotide (WO 96/02555).

In some embodiments of the vaccines described herein, the adjuvant may induce a Th1 type immune response. Suitable adjuvant systems can include, for example, a combination of monophosphoryl lipid A, preferably 3-de-O-acylated monophosphoryl lipid A (3D-MPL) together with an aluminum salt. An enhanced system involves the combination of a monophosphoryl lipid A and a saponin derivative, particularly the combination of 3D-MLP and the saponin QS21 as disclosed in WO 94/00153, or a less reactogenic composition where the QS21 is quenched with cholesterol as disclosed in WO 96/33739. Previous experiments have demonstrated a clear synergistic effect of combinations of 3D-MLP and QS21 in the induction of both humoral and Th1 type cellular immune responses. A particularly potent adjuvant formation involving QS21, 3D-MLP and tocopherol in an oil-in-water emulsion is described in WO 95/17210 and may comprise a formulation.

A vaccine is administered in sufficient amounts to transduce or infect the host cells and to provide sufficient levels of expression to provide an immunogenic benefit without undue adverse effects. Conventional and pharmaceutically acceptable routes of administration include, but are not limited to, intramuscular, intracranial or intraspinal injection. Additional routes of administration include, for example, orally, intranasally, intratracheally, by inhalation, intravenously, subcutaneously, intradermally, or transmucosally.

The dose of a viral vector described herein that can be administered to a subject will depend primarily on factors such as the condition being treated, and the age, weight, and health of the subject. For example, a therapeutically effective dosage of a viral vector to be administered to a human subject generally is in the range of from about 0.1 ml to about 10 ml of a solution containing concentrations of from about 1 × 10e1 to 1 × 10e12 genome copies (GCs) of viruses (e.g., about 1 × 10e9 to 1 × 10e12 GCs). One of the significant benefits of the viral vectors described herein is that a sufficient dose of antigen can be delivered to an individual in a single dose. As used herein, a sufficient dose of antigen refers to an amount of antigen that is sufficient to trigger an active acquired immune response in the individual. Further, another of the significant benefits of the viral vectors described herein is that they can be maintained (e.g., stored) at room temperature without losing efficacy.

The present methods can include administration of a prophylactically effective amount of a vaccine composition as described herein to a subject in need thereof, e.g., a subject who is at risk of developing an infection with SARS-nCoV-2. In some embodiments, the subject has not yet been, but will likely be, exposed to SARS-nCoV-2. In some embodiments, the subject has one or more risk factors associated with a severe infection with SARS-nCoV-2, e.g., pre-existing respiratory (e.g., asthma, COPD), cardiovascular (e.g., PAD, CAD, heart failure), or other (e.g., diabetes) condition that increase the likelihood that if the subject develops a SARS-nCoV-2 infection, that subject is likely to experience a more severe form of the disease, e.g., acute respiratory failure or need for intubation.

A vaccine as described herein can be provided in an article of manufacture (e.g., a kit). An article of manufacture can include a vaccine in a single-dose format or in a multi-dose format. For example, an article of manufacture can include a vaccine in a container (e.g., a vial) or in a vehicle for direct delivery (e.g., a nasal inhaler, an injection syringe). Typically, an article of manufacture also includes instructions for storing the vaccine (e.g., at room temperature) and for delivering or administering the vaccine (e.g., in a single dose).

AAVCOVID novel nucleic acids are provided herein (see, for example, SEQ ID NOs: 1, 2, 3, 4, 9, 11, 13, 15, 16, 17, 18, 23, 25, 27, or 29). As used herein, nucleic acids can include DNA and RNA, and includes nucleic acids that contain one or more nucleotide analogs or backbone modifications. A nucleic acid can be single stranded or double stranded, which usually depends upon its intended use. Also provided herein are novel AAVCOVID polypeptides (see, for example, SEQ ID NOs: 5, 10, 12, 14, 22, 24, 26, or 28).

Also provided are nucleic acids and polypeptides that differ from SEQ ID NOs: 1, 2, 3, 4, 9, 11, 13, 15, 16, 17, 18, 23, 25, 27, or 29 and SEQ ID NOs: 5, 10, 12, 14, 22, 24, 26, or 28, respectively. Nucleic acids and polypeptides that differ in sequence from SEQ ID NOs: 1, 2, 3, 4, 9, 11, 13, 15, 16, 17, 18, 23, 25, 27, or 29 and SEQ ID NOs: 5, 10, 12, 14, 22, 24, 26, or 28 can have at least 80% sequence identity (e.g., at least 85%, 90%, 95%, or 99% sequence identity) to SEQ ID NOs: 1, 2, 3, 4, 9, 11, 13, 15, 16, 17, 18, 23, 25, 27, or 29 and SEQ ID NOs: 5, 10, 12, 14, 22, 24, 26, or 28, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

In addition to the nucleic acids and polypeptides disclosed herein (i.e., SEQ ID NOs: 1, 2, 3, 4, 9, 11, 13, 15, 16, 17, 18, 23, 25, 27, or 29), the skilled artisan will further appreciate that changes can be introduced into a nucleic acid molecule, thereby leading to changes in the amino acid sequence of the encoded polypeptide. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Production of Viral Vectors

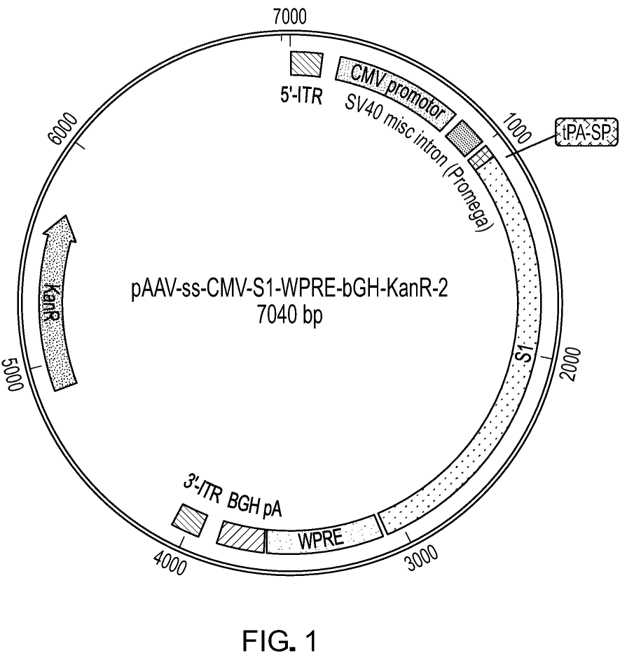

The AAVCOVID strategy is relatively straightforward namely to overexpress SARSnCoV-2019 S antigen via small dose IM injection. This approach is inspired by the fact that (a) MERS coronavirus infection led to long live S protective antibodies and (b) emerging data form SARS-nCoV2 NHP models demonstrate S antibody responses. Three lead candidates are in development. FIG. 1 illustrates (1) a ssAAV full length S, (2) a ssAAV S1 subdomain-expressing vector candidate, which is secreted, and (3) a scAAV secreted RBD subdomain AAV. Designs were based on AAV packaging restrictions (e.g. shorter than SV40 in full length construct), potency considerations (e.g. WPRE element to extend mRNA half-life, use of scAAV which provides a 10-fold higher level and faster onset of expression but only for constructs <2.5 kb). Subdomains were selected based on known antigenicity mapping and available structural information, as well as the prior vaccine work on SARS-CoV 117. IM route was selected based on broad clinical applicability, however similar constructs are evaluated in parallel via intranasal and subcutaneous routes. Prime-boost regimens are evaluated, including heterologous ones through other vaccine agents available through GBCPR.

Viral vectors were produced in the Gene Transfer Vector Core (GTVC), tested for immunogenicity (serum and broncho-alveolar lavage fluid) at a high dose of 10e11 viral particles (vp) in mice and challenged in ferrets (University of Laval BSL3).

Clinical candidates progress to NHP dosing studies 10e9 - 10e12 vp (GTP, UPenn) and CMC characterization (GTVC) is performed in parallel to longer term murine studies for durability of the antibody levels and longer-term safety assessments. The dosing and CMC data package inform clinical study design.

### Example 2—Production of Viral Vectors

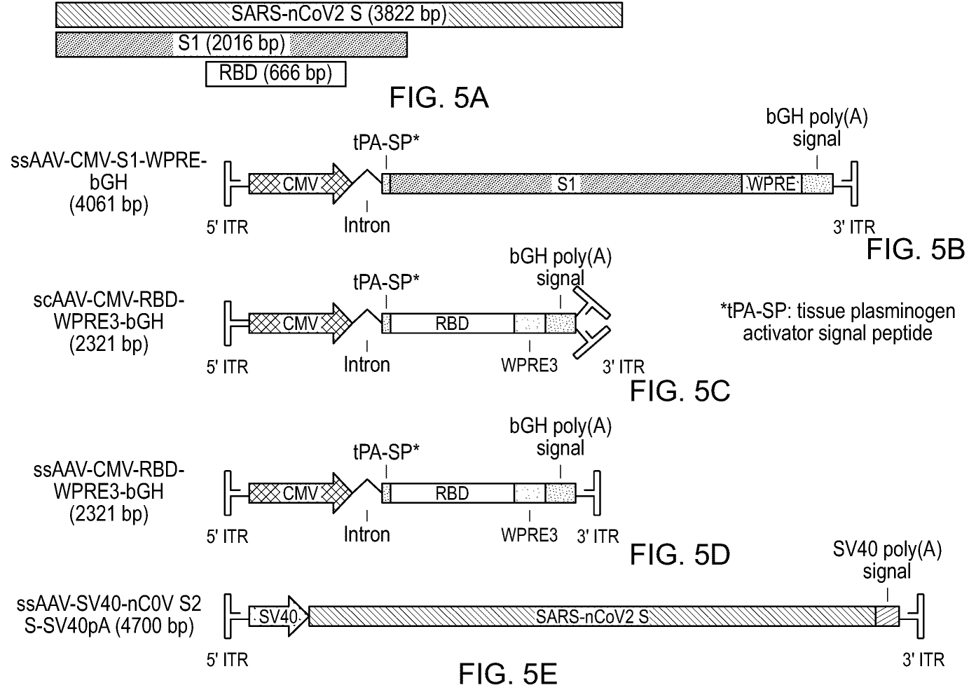

Construct 1, designated pAAV-ss-CMV-S1-WPRE-bGH-KanR-2 (“pK.S1-2”), is shown in FIG. 1. The sequence of the transgene sequence is shown (SEQ ID NO:1), and then a schematic of the transgene within the viral vector is shown. The color coding in the sequence corresponds to the color coding in the schematic of the vector. FIG. 5B shows a schematic of the characteristics of the vector produced with Construct 1.

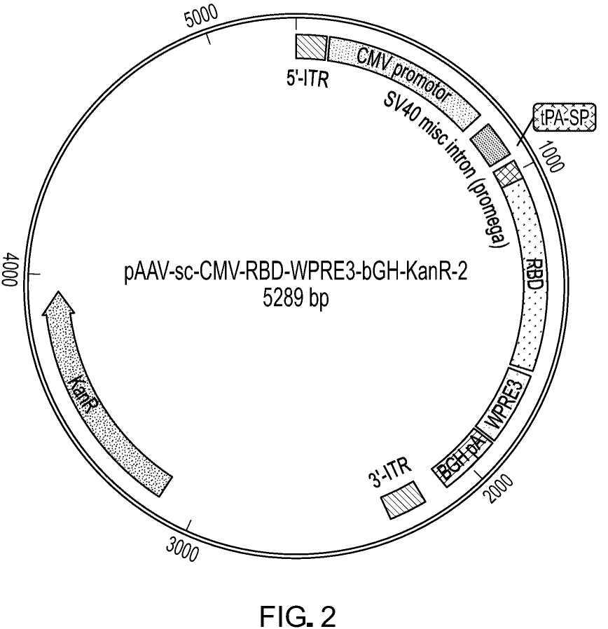

Construct 2, designated pAAV-sc-CMV-RBD-WPRE3-bGH-kanR-2 (“pK.sc-RBD-2”), is shown in FIG. 2. The sequence of the transgene sequence is shown (SEQ ID NO:2), and then a schematic of the transgene within the viral vector is shown. The color coding in the sequence corresponds to the color coding in the schematic of the vector. FIG. 5C shows a schematic of the characteristics of the vector produced with Construct 2.

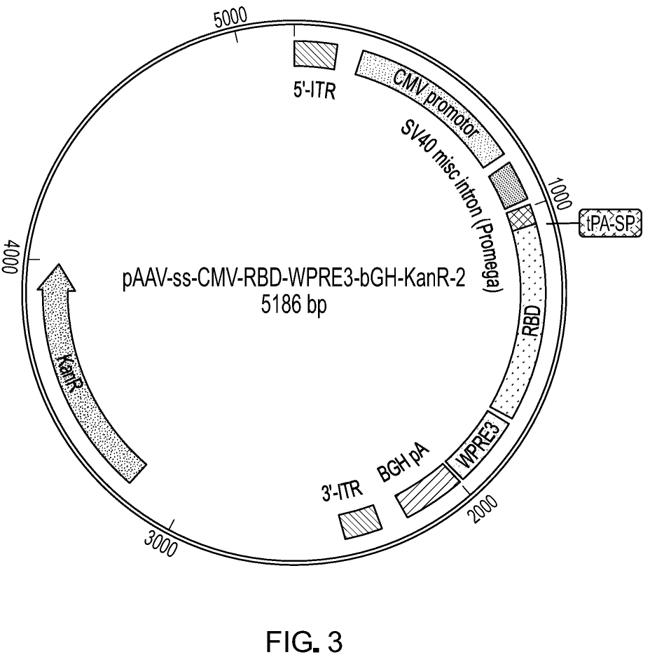

Construct 3, designated pAAV-ss-CMV-RBD-WPRE3-bGH-kanR-2 (“pK.ss-RBD-2”), is shown in FIG. 3. The sequence of the transgene sequence is shown (SEQ ID NO:3), and then a schematic of the transgene within the viral vector is shown. The color coding in the sequence corresponds to the color coding in the schematic of the vector. FIG. 5D shows a schematic of the characteristics of the vector produced with Construct 3.

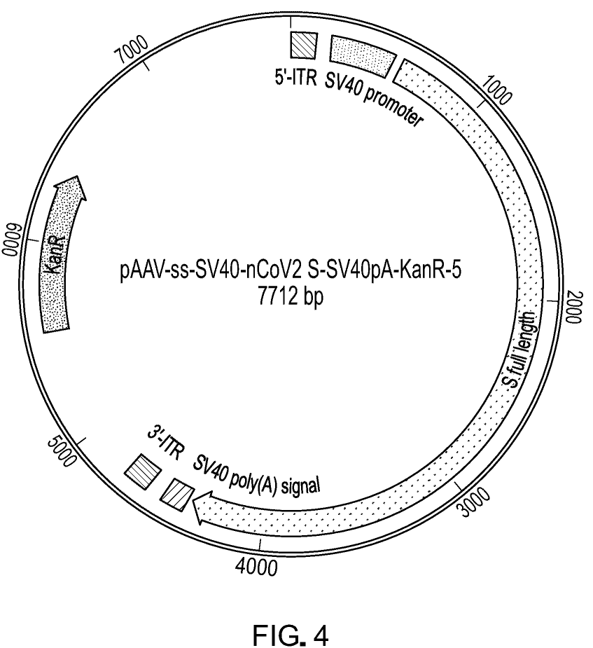

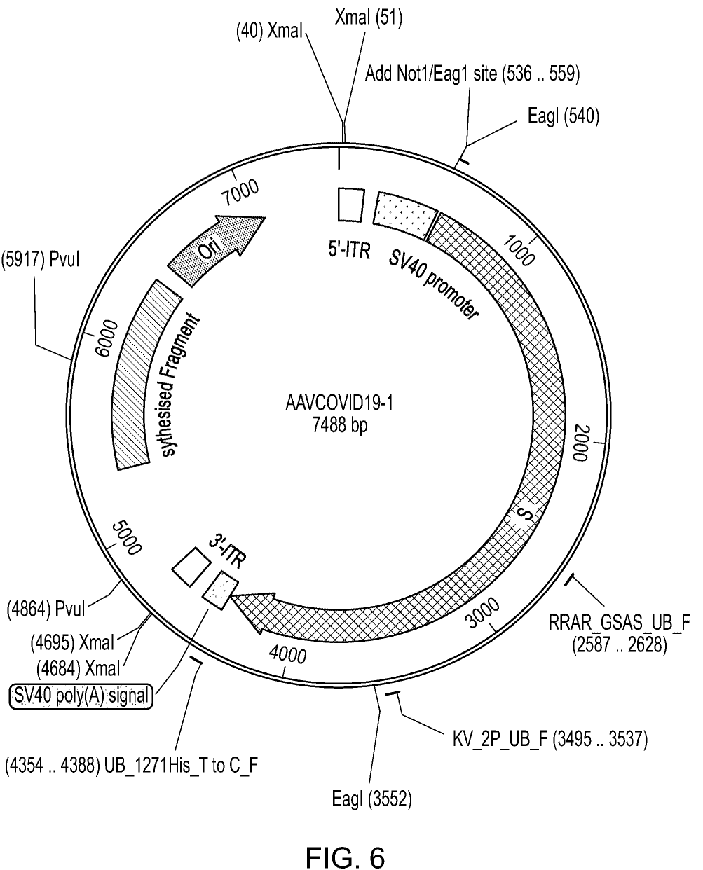

Construct 4, designated pAAV-ss-SV40-nCoV2 S-SV40pA-KanR-5 (“pK.FL-5”), is shown in FIG. 4. The sequence of the transgene sequence is shown (SEQ ID NO:4), and then a schematic of the transgene within the viral vector is shown. The color coding in the sequence corresponds to the color coding in the schematic of the vector. FIG. 5E shows a schematic of the characteristics of the vector produced with Construct 4, and FIG. 6 shows the amino acid sequence of Construct 4.

All the constructs described below start with the sequence of Construct 4 but carry several protein-stabilizing mutations to improve nCoV-2 Spike protein expression and immunogenicity. Specifically, the mutations described below result in the stabilization of the pre-fusion state of the SPIKE protein, a conformational state that must be recognized by the subject’s antibodies to protect against SARS-nCoV-2 infection. All the residues and domains named below are depicted in FIG. 6.

Constructs 5, 6, 7, 8, 9, 10, and 11 consist of the same sequence of Construct 4 but carry several protein-stabilizing mutations to improve nCoV-2 S expression and immunogenicity. Specifically, the mutations described below result in the stabilization of the pre-fusion state of the SPIKE protein, a conformational state that must be recognized by the subject’s antibodies to protect against SARS-nCoV-2 infection. Constructs 5, 6, and 7 are designed to be furin cleavage mutants, in which the amino acid sequence R682RAR685↓S is mutated to G682SAS685 (Construct 5), to G682GSG685 (Construct 6), or to I682LR684 (Construct 7) (Kirchdoerfer et al Nature 2016, 531(7592):118-21; Walls et al., Cell, 2019, 176(5):1026-39; Wrapp et al., Science, 2020, 367(6483):1260-3). Construct 8 carries two proline substitutions at positions 986 and 987 (K986P and V987P) that increase the rigidity of the loop between the heptad repeat 1 (HR1) and the central helix, avoiding a premature change to the fusion protein conformation (Pallesen et al., PNAS, 2017, 114(35):E7348-57; Wrapp et al., Science, 2020, 367(6483):1260-3). Construct 9 combines the modifications made in Constructs 5 and 8; the mutations in the furin cleavage site from R682RAR685↓S to G682SAS685, and the K986P and V987P substitutions. Construct 10 is a combination of the changes made in Constructs 6 and 8; the mutation in the furin cleavage site from R682RAR685↓S to G682GSG685, and the K986P and V987P substitutions. Construct 11 combines mutations of Construct 7 and 8, furin cleavage site mutated to I682LR684 and K986P and V987P substitutions.

Constructs 12, 13, 14, 15, 16, 17, 18 and 19 include the same sequences described in Constructs 4, 5, 6, 7, 8, 9, 10 and 11, respectively, but the transmembrane domain and the cytoplasmic domain of the SPIKE protein were removed by the addition of an early stop codon (G1219Ter). These versions are secreted ectodomains that have the ability to trimerize.

Constructs 20, 21, 22, 23, 24, 25, 26 and 27 include the sequences in Constructs 12, 13, 14, 15, 16, 17, 18 and 19, respectively, but the signal peptide (first 13 residues of the protein) has been change to the tissue plasminogen activator signal peptide (tPA-SP) to improve protein secretion (Wang et al., 2011, Appl. Microbiol. Biotech.).

Constructs 28, 29, 30, 31, 32, 33, 34 and 35 include the same sequences described in Constructs 4, 5, 6, 7, 8, 9, 10 and 11, respectively, but the transmembrane domain and the cytoplasmic domain have been substituted by the GCN4 trimerization domain (IKRMKQIEDKIEEIESKQKKIENEIARIKKIK (SEQ ID NO:6)) to improve proper trimarizetion of SPIKE ectodomain (Walls et al., Nature, 2016, 531(7592):114-7; Walls et al., Prot. Science, 2017, 26(1):113-21).

Constructs 36, 37, 38, 39, 40, 41, 42 and 43 include the same sequences described in Constructs 4, 5, 6, 7, 8, 9, 10 and 11, respectively, but the transmembrane domain and the cytoplasmic domain have been substituted by the T4 fibritin trimerization domain (GSGYIPEAPRDGQAYVRKDGEWVLLSTFL (SEQ ID NO:7)) to improve proper trimarizetion of SPIKE ectodomain (Pallesen et al., PNAS, 2017, 114(35):E7348-57; Walls et al., Cell, 2020, doi: 10.1016/j.cell.2020.02.058; Wrapp et al., Science, 2020, 367(6483):1260-3)

Constructs 44, 45, 46, 47, 48, 49, 50 and 51 include the same sequences described in Constructs 4, 5, 6, 7, 8, 9, 10 and 11, respectively, but the transmembrane domain and the cytoplasmic domain have been substituted by a modified isoleucine zipper that has four glycosylation motif (GGTGGNGTGRMKQIEDKIENITSKIY NITNEIARIKKLIGNRT (SEQ ID NO:8)) to improve proper trimarizetion of SPIKE ectodomain and reduce immunogenicity of the trimerization domain (Sliepen et al., 2015, J. Biol. Chem., 290(12):7436-42).

Constructs 52, 53, 54, 55, 56, 57, 58, 59, 60, 61, 62, 63, 64, 65, 66, 67, 68, 69, 70, 71, 72, 73, 74 and 75 include the same sequences described in Constructs 28, 29, 30, 31, 32, 33, 34, 35, 36, 37, 38, 39, 40, 41, 42, 43, 44, 45, 46, 47, 48, 49, 50 and 51, respectively, but the signal peptide (first 13 residues of the protein) has been change to the tissue plasminogen activator signal peptide (tPA-SP) to improve protein secretion (Wang et al., 2011, Appl. Microbiol. Biotech.).

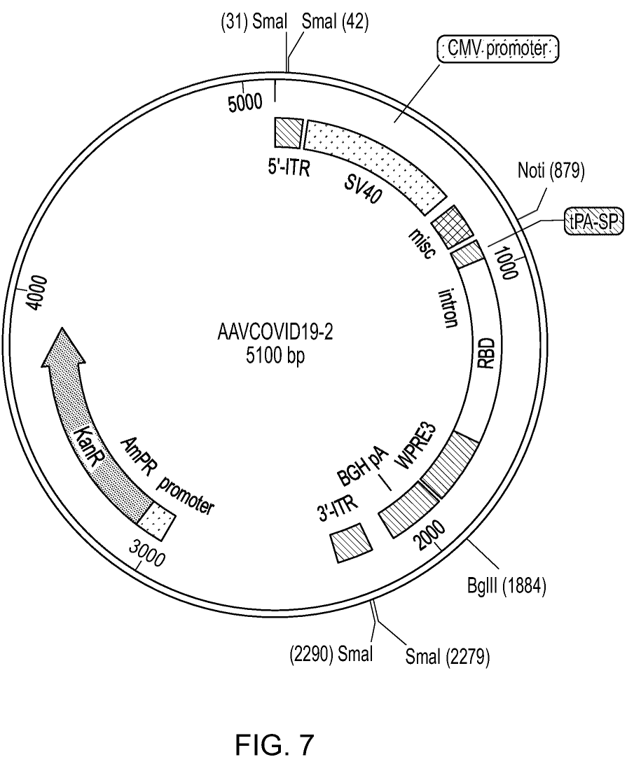

For example, AAVCOVID19-1 features a human codon optimized ORF as well as stabilizing mutations to make full-length spike protein (RRAR682-685 to GSAS682-685 for Furin enzyme cleavage site, KV986-987 to PP986-987 (bold and underlined in the sequence shown in FIG. 7; SEQ ID NOs: 9 and 10).

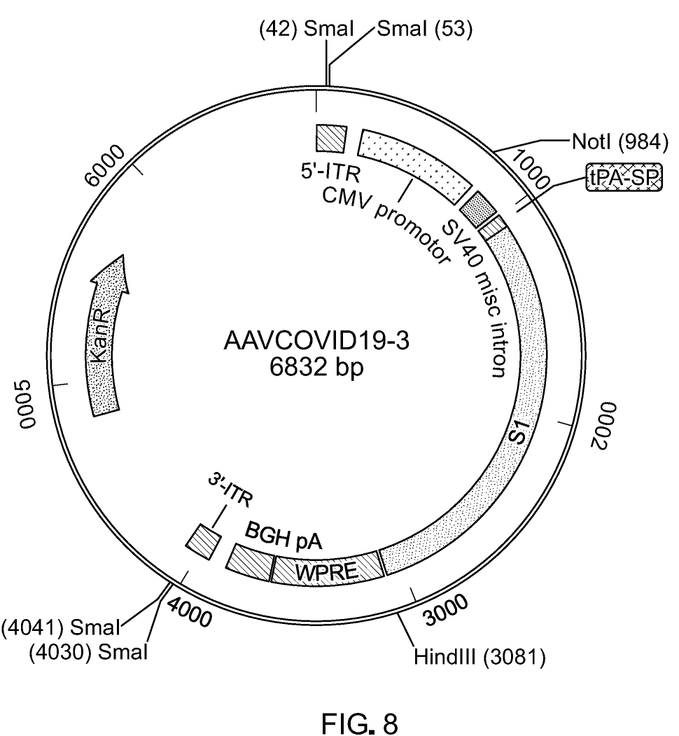

For example, AAVCOVID19-2 features a human codon optimized ORF, attachment of the human tissue plasminogen activator signal peptide (tPA-SP) (bold and underlined in the sequence shown in FIG. 8), and includes a self-complimentary sequence of the gene of interest. SEQ ID NOs: 11 and 12.

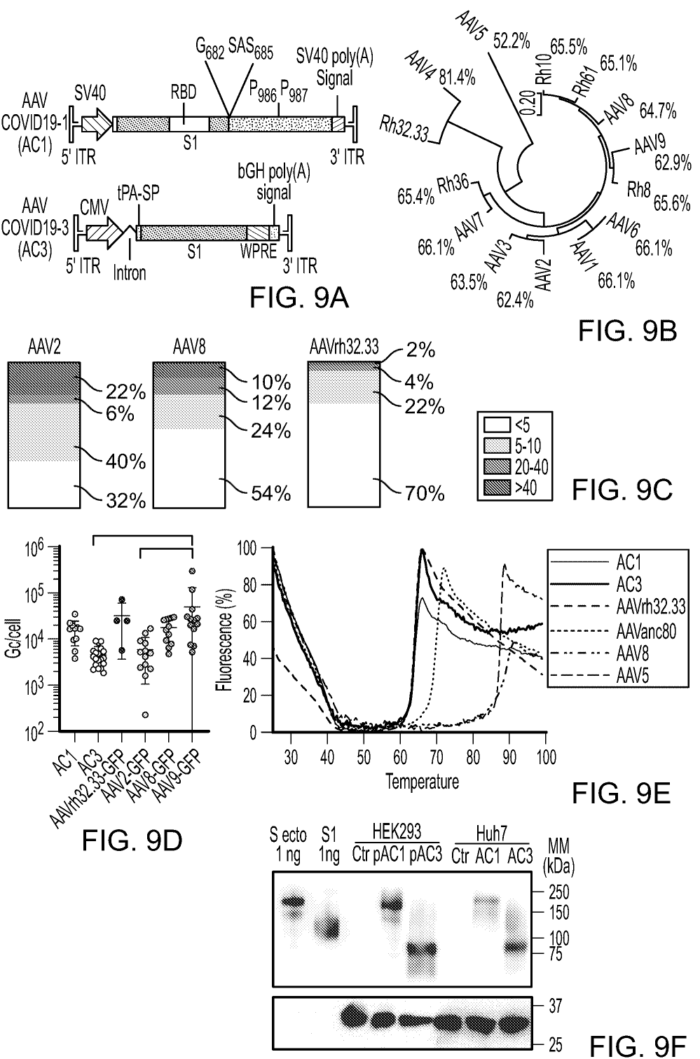

For example, AAVCOVID19-3 features a human codon optimized ORF and attachment of the human tissue plasminogen activator signal peptide (tPA-SP) (bold and underlined in the sequence shown in FIG. 9; SEQ ID NOs: 13 and 14)

### Example 3—Vaccine Candidates

wo AAV-based vaccine candidates were tested: AAVCOVID19-1 (AC1) and AAVCOVID19-3 (AC3) (FIG. 9A) (GenBank Accession Nos. MW408785 and MW408786, respectively). AC1 is an AAVrh32.33 vector that expresses the codon optimized, pre-fusion stabilized (furin cleavage site mutated to G682SAS685 and P986P987 substitutions) full length SARS-CoV-2 Spike protein under the control of an SV40 promoter. AC1 carries a short SV40 polyadenylation signal (poly-A). AC3 is an AAVrh32.33 that carries the secreted S1 subunit of SARS-CoV-2 Spike with the tissue plasminogen activator signal peptide (tPA-SP) whose expression is driven by the CMV promoter. AC3 has two more regulatory elements: a woodchuck hepatitis virus posttranscriptional regulatory element (WPRE) and the bovine growth hormone polyadenylation signal (poly-A).

### Example 4—Small-Scale Production of Vaccine Candidates

Research-grade, high-titer vectors were produced, purified, and titrated by the MEEI/ SERI Gene Transfer Vector Core (vdb-lab.org/vector-core/ on the World Wide Web). Small-scale vector preparations were generated by polyethylenimine or PEI (Polysciences, Cat #24765-2) triple transfection of AC1 or AC3 ITR-flanked transgene, pKan2/rh32.33 (AAV2 rep and AAVrh32.33 capsid construct), and pALD-X80 adenoviral helper plasmid in a 1:1:2 ratio, respectively, in HEK293 cells. DNA was transfected in 10-layer HYPERFlasks using a PEI-Max/DNA ratio of 1.375:1 (v/w). 3 days after transfection, vectors were harvested from the HYPERFlasks using Benzonase (EMD Millipore, Cat. #1016970010) to degrade DNA/RNA. 24 hours after harvesting, the vectors were concentrated by tangential flow filtration and purified by iodixanol gradient ultracentrifugation as previously described (Lock et al., 2010, Human Gene Ther., 21:1259-71). Vaccine candidates were quantified by ddPCR according to a previously published protocol (Sanmiguel et al., 2019, Quantitative and Digital Droplet-Based AAV Genome Titration, Methods Mol. Biol., Clifton, NJ, 1950). Capsid stability was assessed by AAV-ID (Pacouret et al., 2017, Mol. Ther: J. Am. Soc. Gene Ther., 25).

### Example 5—TaqMan™ Assay Design for SARS-CoV-2 Spike Detection

The codon optimized SARS-CoV-2 receptor binding domain (RBD) of AAVCOVID vaccine candidates was used as a target for droplet digital PCR (ddPCR)/real-time PCR (qPCR) quantifications. The sequence was checked for secondary structures using the mfold application of the UNAfold software package (Zuker, 2003, Nuc. Acids Res., 31:3406-15) at the PCR annealing temperature and TaqMan buffer salt concentrations. Internal repeats were avoided by mapping against the entire codon optimized SARS-CoV-2 S gene of AAVCOVID candidates using the REPuter application (Kurtz et al., 2001, Nuc. Acids Res., 29:4633-42). The 5′-end of the gene was selected as PCR target based on these analyses. The oligo sequences used were the following: forward primer, GTG CAG CCA ACC GAG (0.43 µM final concentration (SEQ ID NO: 19)); reverse primer, ACA CCT CGC CAA ATG G (1.125 µM final concentration (SEQ ID NO: 20)), and TaqMan® probe 6FAM- TCT ATC GTG CGC TTT C-MGBNFQ (0.25 µM final concentration (SEQ ID NO: 21)). The final concentration and Tm’s of primers were determined using the DINAMelt application of the UNAfold software package (Markham and Zuker, 2005, Nuc. Acids Res., 33:W577-81; Markham and Zuker, 2008, Methods Mol. Biol., 453:3-31) and set to hybridize the target with a Tm of just under 60° C. (59.0-59.9° C.) for high specificity. The PrimerExpress™ software (Applied Biosystems™) was used to determine the Tm of the MGB probe (Kutyavin et al., 2000, Nuc. Acids Res., 28:655-61). The resulting 67 bp amplicon was inspected for specificity via NCBI BLAST® using the somewhat similar algorithm in the suite against human, NHP, mouse, ferret, and betacoronavirus databases and determined to be highly specific for our vaccine candidates. No significant matches were found against the RBD oligonucleotides used.

### Example 6—In Vitro Expression Studies

10e5 HEK293 cell/well were seeded in 12-well plates (Corning, MA, USA) plates and incubated at 37° C. overnight. The following day, cells were transfected with 2 µg of AAVCOVID19-1 (pAC1) and AAVCOVID19-3 (pAC3) plasmids using PEI-Max. Cells were harvested 24 and 72 hours after transfection for mRNA and Western blot (WB) expression analyses, respectively. In addition, 5 × 10e4 HuH7 cell/well were seeded in 12-well plates and incubated overnight at 37° C. On the following day, Adenovirus 5 WT (Ad5) was added to the cells at a MOI of 20 pfu/cell. 2 hours later, media was removed, and cells infected with a MOI of 5 × 10e5 of AC1 or AC3. Cells were harvested 72 hours later for WB analysis.

Transfection and transduction samples were also collected for RNA gene expression analyses. Total RNA was extracted via Trizol™ reagent (Invitrogen™) and quantified using a Qubit™ fluorometer (Invitrogen™). 7.5 µg of Total RNA was DNase-I treated using the Turbo DNA-free™ kit (Invitrogen™). About 1.4 µg of DNase-treated total RNA was set aside for reverse transcription against (-)RT controls using the high capacity cDNA reverse transcription kit (Thermo Fisher™). Codon optimized RBD gene expression was assessed against a cells only control using qPCR and normalized to human 18S rRNA gene levels by the delta delta Ct method (Livak and Schmittgen, 2001, Methods, 25:402-8).

### Example 7—Detection of Spike Antigens by Western Blot (WB)

Cell lysates were obtained by diluting cell pellets in NuPAGE™ LDS Sample Buffer (4X) (Thermo Fisher Scientific, Cat# NP0007) and incubating at 99° C. for 5 minutes, separated by electrophoresis in NuPAGE 4-12% polyacrylamide gels (Thermo Fisher Scientific, Cat #NP0321PK2) and then transferred to PVDF membranes. The membranes were probed with an anti-SARS-CoV-2 RBD rabbit polyclonal antibody (Sino Biological Inc., Cat. #40592-T62) followed by a goat anti-rabbit HRP-conjugated secondary antibody (Thermo Fisher Scientific, Cat. #A16110, RRID AB_2534782). Membranes were developed by chemiluminescence using the Immobilon Western Chemiluminescent HRP Substrate (Millipore, Cat. #WBKLS0500) and recorded using ChemiDoc MP Imaging System (Bio-Rad). An anti-GAPDH antibody (Cell Signaling Technology, Cat. #2118, RRID:AB_561053) was used as loading control.

### Example 8—Mouse Studies

All the mouse studies were performed in compliance with the Schepens Eye Research Institute IACUC. BALB/c, C57BL/6 or C57BL/6 diet-induced obese (DIO) animals were intramuscularly (right gastrocnemius muscle) treated at 10e10 gc/mouse or 10e11 gc/mouse. Animals were kept in standard diet and C57BL/6 DIO were fed a high-fat diet (Research Diets, Cat. #D12492i). Serum samples were obtained by submandibular bleeds for humoral immune response analyses. At necropsy, several tissues were collected for analysis of vector presence and transgene expression.

### Example 9—NHP Study

All animal procedures were approved by the Institutional Animal Care and Use Committee of the Children’s Hospital of Philadelphia. Rhesus macaques (Macaca mulatto) that screened negative for viral pathogens including SIV (simian immunodeficiency virus), STLV (simian-T- lymphotrophic virus), SRV (simian retrovirus), and B virus (macacine herpesvirus 1) were enrolled on the study. Animals were housed in an AAALAC International-accredited non-human primate research in stainless-steel squeeze back cages, on a 12-hour timed light/dark cycle, at temperatures ranging from 64-79° F. (18-26° C.). Animals received varied enrichment such as food treats, visual and auditory stimuli, manipulatives, and social interactions throughout the study. Four 3 to 7 year-old Rhesus macaques (Macaca mulatto) were treated with the clinical candidates (n=2 per vector, 1 female and 1 male) intramuscularly at a dose of 10e12 gc/animal. Serum and PBMC samples were obtained in regular intervals for several analyses of immunogenicity against SARS-CoV-2 Spike and AAVrh32.33. Serum chemistry, hematology, and coagulation analyses were performed by Antech Diagnostics. Serum was also collected for cytokine analyses which were performed by the University of Pennsylvania’s Human Immunology Core using a Non-Human Primate Cytokine Panel kit (MilliporeSigma, Cat. #PCYTMG-40K-PX23) on a Bio-Plex 200 instrument (Bio-Rad) according to the manufacturer’s protocol.

### Example 10—Human Samples

Blood was collected from 60 patients with nasopharyngeal PCR-confirmed SARS-CoV-2 infection stratified by disease severity. Plasma was separated and stored at negative 80° C. until assessed. Human subject investigation was approved by the institutional Review Board of the Massachusetts General Hospital.

### Example 11—SARS-CoV-2 Spike-Binding Antibody Detection ELISA

Nunc MaxiSorp™ high protein-binding capacity 96 well plates (Thermo Fisher Scientific, Cat. #44-2404-21) were coated overnight at 4° C. with 1 ug/ml SARS-CoV-2 RBD, SARS-CoV-2 ectodomain (LakePharma, Cat. #46328) or SARS-CoV-1 RBD diluted in phosphate-buffered saline (PBS). The next day the plates were washed with PBS-Tween 20 0.05% (Sigma, Cat. #P2287-100 ML) using the Biotek 405 TS Microplate washer. Each plate was washed five times with 200 µl wash buffer and then dried before the next step. Following the first wash, 200 µl of Blocker Casein in PBS (Thermo Fisher Scientific, Cat. #37528) were added to each well and incubated for 2 hours at RT. After blocking, serum samples were serially diluted in blocking solution starting into 1:100 dilution. After an hour of incubation, the plates were washed and 100 µl of secondary Peroxidase AffiniPure Rabbit Anti-Mouse IgG (Jackson ImmunoResearch, Cat. #315-035-045, RRID: AB_2340066) antibody diluted 1:1000 in blocking solution or rabbit Anti-Monkey IgG (whole molecule)-Peroxidase antibody (Sigma-Aldrich, Cat. #A2054, RRID:AB_257967) were added to each well. After one hour of incubation at room temperature, the plates were washed and developed for 3.5 min with 100 µl of Seracare SureBlue Reserve™ TMB Microwell Peroxidase Substrate solution (SeraCare, Cat. #53-00-03). The reaction was then stopped with 100 µl Seracare KPL TMB Stop Solution (SeraCare, Cat. #50-85-06). Optical density (OD) at 450 nm was measured using a Biotek Synergy H1 plate reader. The titer was the reciprocal of the highest dilution with absorbance values higher than four times the average of the negative control wells. For mouse serum SARS-CoV-2 RBD-specific antibody isotyping, the same ELISA was performed but using the secondary antibodies from SBA Clonotyping System-HRP kit (SouthernBiotech, 5300-05, RRID:AB_2796080) diluted accordingly to manufacturer’s instructions.

For NHP isotyping ELISA, plates were coated overnight at 4° C. with 20 ng/well of SARS-CoV-2 Spike protein ectodomain (LakePharma, Cat. #46328) as the capture antigen. Then, the wells were blocked with 5% milk in PBS-Tween 20 0.05% for 2 h and incubated for additional 2 h with 50 µl serially diluted serum samples. Then, horseradish peroxidase (HRP)-conjugated secondary antibody against rhesus IgG1 (NIH Nonhuman Primate Reagent Resource supported by AI126683 and OD 010976 Cat. #PR-7110, RRID:AB_2819310) and IgG4 (NIH Non-human Primate Reagent Resource supported by AI126683 and OD 010976 Cat. #PR-7180, RRID:AB_2819322) for 1 h. After every incubation step, the plates were washed three times with PBS-Tween 20 0.05%. After color development, OD at 450 nm was determined using Biotek Synergy H1 plate reader. The titer was the reciprocal of the highest dilution with absorbance values higher than four times the average of the negative control wells.

### Example 12—Lenti-SARS2 Pseudovirus Production and Titration

Lenti-SARS2 was produced based on a published protocol (Crawford et al., 2020, Viruses, 12:513). Specifically, 50% confluent HEK293T cells were seeded 24 hours prior to transfection in 15 cm plates. The next day, 18 µg of psPAX2, 9 µg of pCMV-SARS2-RRAR_ILR_gp41 and 29 µg of pCMV-Lenti-Luc plasmids were mixed in 3.6 mL of Opti-MEM™ I Reduced Serum media (Gibco, Cat. #31985070) along with 144 µL of PEI Max 40 K (1 mg/mL, pH 6.9-7.1) and mixed thoroughly. The mixture was incubated for 20 minutes at room temperature. Media on cells was aspirated and serum-free DMEM was added to the cells. After 20 mins, the DNA-PEI mixture was added dropwise to the plate and incubated overnight at 37° C. with 5% CO₂. The next day, media was replaced with DMEM with 10% FBS. After 48 hours, the media was collected in a 50 mL conical and centrifuged at 4,000 rpm at 4° C. for 5 minutes to remove cell debris. The supernatant was collected and filtered through 0.45 µm filter, aliquoted and stored at -80° C.

For titration of the pseudovirus, HEK293T cells expressing ACE2 were seeded at 1.5 × 10e4 cells/well in poly-L-Lysine (0.01%) coated 96-well black plates (Thermo Fisher Scientific, Cat. #3904) one day before titration. On the next day, the media was changed to 50 µL DMEM + 10% FBS containing filtered Hexadimethrine bromide at a final concentration of 10 µg/mL. 2-fold serial dilutions (up-to 15 dilutions) of the viral stocks (50 µL) were added to the plate in 6 replicates each and incubated for 48 hours. After 48 hours, cells were lysed with Reporter Lysis Buffer (Promega, Cat. #E4030). These plates were frozen at -80° C. for 60 minutes. Thereafter, they were thawed at 37° C. for 20 mins before starting the luciferase readout. For luciferase substrate buffer, the following reagents were mixed; Tris-HCl buffer at 0.5 M, ATP at 0.3 mM, MgC1₂ at 10 mM, Pierce™ Firefly Signal Enhancer (Thermo Fisher Scientific, Cat. #16180), D-luciferin 150 µg/mL (PerkinElmer, Cat. #122799). Biotek Synergy H1 Plate reader was used for luminescence readout. For pseudovirus neutralization assay, a final dilution of the virus stock targeting relative luminescence units (RLU) of 1800-1100 was used, which was approximately 200-fold higher than background signal obtained in untreated cells.

### Example 13—Pseudovirus Neutralization Assay

HEK293T cells expressing ACE2 were seeded at 1.5 × 10e4 cells / well in poly-L-Lysine (0.01%) coated 96-well black plates. The following day, 50 µL of DMEM + 10% FBS media containing Hexadimethrine bromide (final concentration 10 µg/mL) was added to the cells. Serum samples were heat-inactivated at 56° C. for 1 hour. Serum samples were then serially diluted (2-fold) for 10 dilutions in DMEM with 10% FBS with initial dilution of 1:40 for mouse serum and 1:10 dilution for NHP serum. Thereafter, Lenti-SARS2 pseudovirus was added to each dilution and incubated at 37° C. for 45 minutes. The serum and virus mixture was added to the cells and incubated at 37° C. with 5% CO₂ for 48 hours. An anti-SARS-CoV-2 Spike monoclonal neutralizing antibody (GenScript, Cat. #6D11F2) was used as a positive control. Cells without serum and virus were used as negative control. After 48 hours, cells were lysed and luciferase measured as described above. Neutralizing antibody titers or 50% inhibitory concentration in the serum sample (EC₅₀ or ID₅₀) were calculated as the reciprocal of the highest dilution showing less RLU signal than half of the average RLU (maximum infectivity) of Virus Control group (cells + virus, without serum).

### Example 14—Plaque Reduction Neutralization Test (PRNT) of SARS-CoV-2

Depending on the volume available, mouse or NHP sera were serially diluted twofold from an initial dilution of either 1:12.5 or 1:25 for ten dilutions in Dulbecco’s Phosphate Buffered Saline (DPBS, Gibco). Each dilution was incubated at 37° C. and 5% CO₂ for 1 hour with an equal volume of 1000 plaque forming units/ml (PFU/ml) of SARS-CoV-2 (isolate USA-WA1/2020) diluted in DMEM (Gibco) containing 2% fetal bovine serum (Gibco) and antibiotic-antimycotic (Gibco). Controls included DMEM containing 2% fetal bovine serum (Gibco) and antibiotic-antimycotic (Gibco) only as a negative control, 1000 PFU/ml SARS-CoV-2 incubated with DPBS, and 1000 PFU/ml SARS-CoV-2 incubated with DMEM. Two hundred microliters of each dilution or control were added to confluent monolayers of NR-596 Vero E6 cells in triplicate and incubated for 1 hour at 37° C. and 5% CO₂. The plates were gently rocked every 5-10 minutes to prevent monolayer drying. The monolayers were then overlaid with a 1:1 mixture of 2.5% Avicel® RC-591 microcrystalline cellulose and carboxymethylcellulose sodium (DuPont Nutrition & Biosciences) and 2X Modified Eagle Medium (Temin’s modification, Gibco) supplemented with 2X antibiotic-antimycotic (Gibco), 2X GlutaMAX (Gibco) and 10% fetal bovine serum (Gibco). Plates were incubated at 37° C. and 5% CO₂ for 2 days. The monolayers were fixed with 10% neutral buffered formalin and stained with 0.2% aqueous Gentian Violet (RICCA Chemicals) in 10% neutral buffered formalin for 30 minutes, followed by rinsing and plaque counting. The half maximal inhibitory concentrations (EC₅₀ or ID₅₀) were calculated using GraphPad Prism 8.

### Example 15—IFN-Gamma and IL-4 ELISPOT Assay and Cytokine Secretion in Murine Splenocytes

Splenocytes were obtained by grinding murine spleens with 100 µm cell strainers, followed by treatment with Ammonium Chloride-Potassium (ACK) lysis buffer (Gibco) to lyse the red blood cells. The isolated cells were then suspended in complete RPMI-1640 medium (Gibco) supplemented with 10% FBS and counted for the following experiments.

IFN-gamma and IL-4 ELISPOT for mice was measured as previously described (Wang et al., 2019, Gut, 68:1813-9). Briefly, 96-well PVDF plates (Millipore) were pre-coated with 10 µg/ml anti-mouse IFN-gamma ELISPOT capture antibody (BD Biosciences, Cat. #551881, RRID:AB_2868948) or 4 µg/ml anti-mouse IL-4 ELISPOT capture antibody (BD Biosciences, Cat. #551878, RRID:AB_2336921) at 4° C. overnight, and then blocked with complete RPMI-1640 medium for 3 hours at 37° C. One million of freshly isolated splenocytes were seeded into the pre-coated plates and stimulated with S1 and S2 peptides pools (GenScript) with a final concentration of 1 µg/ml of each peptide diluted in RPMI-1640 supplemented with 10% FBS and incubated for 48 hours at 37° C. with 5% CO₂. Each peptide pool, consisting of 15-mers peptides overlapping by 10 amino acids, spanning the entire SARS-CoV-2 Spike protein S1 or S2 subunits. Control wells contained 5×10⁵ cell stimulated with DMSO diluted in RPMI-1640 supplemented with 10% FBS (negative control) or 2 µg/ml concanavalin A (positive control). Subsequently, the plates were washed and incubated with biotin-conjugated mouse IFN-gamma ELISPOT Detection Antibody (BD Biosciences, Cat. #551881, RRID:AB_2868948) and 4 µg/ml biotin-conjugated mouse IL4 detection antibody (BD Biosciences, Cat. #551878, RRID:AB_2336921) at room temperature for 3 hours and followed by streptavidin-HRP (dilution 1:1000, Sigma-Aldrich, Cat. #18-152) for 45 minutes. After washing, 100 µL/well of NBT/BCIP substrate solution (Promega, Cat. #S3771) were added and developed for 15-30 min until distinct spots emerged. The cytokine-secreting cell spots were imaged and counted on AID EliSpot reader (Autoimmun Diagnostika GmbH).

2×10e6 freshly isolated splenocytes were seeded into 96-well plates and stimulated with 1 µg/ml of peptides from S1 and S2 pool as described previously at 37° C. for 48 hours. Then the supernatants were collected and cytokine levels were measured by a Luminex cytokine assay by SBH Sciences.

### Example 16—Detection of Circulating S1 in NHP Sera by Sandwich ELISA

A monoclonal anti-SARS-CoV-2 RBD capture antibody (GenScript, Cat. #5B7D7) was coated on Nunc Maxisorp ELISA plates (Thermo Fisher Scientific, Cat. #44-2404-21) at 2.5 µg/mL final concentration in Sodium Bicarbonate buffer (Sigma-Aldrich, Cat. #SRE0034). The plate was incubated at 4° C. overnight. All washes were performed 5X with PBS-Tween-20 0.05%. On the following day, plates were washed and blocked for 2 hours with Casein Buffer (Thermo Fisher Scientific, Cat. #37528). Then, NHP sera were added in duplicates at 1:5 dilution in blocking buffer. A blank consisting of the blocking buffer and a standard curve ranging from 5000 pg/mL to 78.25 pg/mL of S1 antigen (GenScript, Cat. #Z03501) in blocking buffer were also added in duplicates on the plate followed by incubation at room temperature for 1 hour. Then, biotinylated detection antibody (GenScript, Cat. #5E10G8-Biotin) was added at 1 µg/mL final concentration in blocking buffer and plate was incubated at room temperature for 1 hour. Finally, 1:5000 final dilution of Streptavidin-HRP (Sigma-Aldrich, Cat. #18-152) was added to the plate. After completing incubation of 1 hour at room temperature, the plate was washed. 100 µL of TMB substrate (SeraCare, Cat. #5120-0081) was added to plates and color was developed for 3 mins 30 secs, after which 100 µL of Stop Solution (SeraCare, Cat. #5150-0021) was added to stop the reaction and plates were read at 450 nm and 670 nm using Biotek Synergy H1 hybrid plate reader. Absorbance at 670 nm was subtracted from 450 nm, and then corrected with absorbance of the blank. Linear regression was used to calculate the standard curve formula and S1 concentration (pg/mL) was calculated by extrapolation.

### Example 17—IFN-Gamma ELISPOT Assay in NHP PBMCs

Peripheral blood T cell responses against AC1, AC3 and the AAVrh32.33 capsid were measured by interferon gamma (IFN-gamma) enzyme-linked immunosorbent spot (ELISPOT) assays according to previously published methods (Calcedo et al., 2018, Hum. Gene Ther. Methods, 29:86-95). Peptide libraries specific for AAVrh32.33 capsid as well as the AC1 and AC3 transgenes were generated (15-mers with a 10 amino acid overlap with the preceding peptide; Mimotopes, Australia). More specifically, the AAVrh32.33 capsid peptide library was divided into three peptide pools, A, B and C. Pool A contained peptides 1-50, Pool B contained peptides 51-100 and Pool C contained peptides 101-145. For the AC1 and AC3 peptide libraries, peptides specific to each protein were pooled separately from those peptide sequences shared between the two proteins. The AC1 peptide library contained Pool A (peptides 1-2, 136-173); Pool B (peptides 174-213); and Pool C (peptides 214-253). The AC3 Peptide Library consisted of Pool A only (peptides 254-257). The AC1 & AC3 Shared Peptides also contained three peptide pools; Pool A (peptides 258-259; 3-44), Pool B (peptides 45-90) and Pool C (peptides 91-135). Peptides were dissolved in dimethyl sulfoxide (DMSO) at a concentration of 100 mg/mL, pooled, aliquoted and stored at -80° C. They were used at a final concentration in the assay of approximately 2 µg/mL. The positive response criteria for the IFN-gamma ELISPOT was greater than 55 spot forming units (SFU) per million cells and at least three times greater than the negative control values.

### Example 18—PBMC Stimulation, Flow Cytometry and Gating Strategy

Cryopreserved peripheral blood mononuclear cells (PBMC) were thawed and rested overnight in sterile R10 media (RPMI 1640, Coming), supplemented with 10% fetal bovine serum (Gemini Bio-Products), Penicillin/Streptomycin and L-Glutamine; plus 10 U/mL DNAse I (Roche Life Sciences) at 37° C., 5% CO₂ and 95% humidity incubation conditions. PBMC were stimulated at 200 µl final volume in sterile R10 media. Peptide concentrations for stimulation conditions were 2 µg/ml for AC1/AC3 shared peptide pool A, B and C and AAVrh32.22 peptide pool A, B and C. Co-stimulation was added with peptides: 1 µg/mL anti-CD49d (Clone 9F10, BioLegend, Cat. #304301, RRID:AB_314427) and CD28-ECD (Clone CD28.2, Beckman Coulter, Cat. #6607111, RRID:AB_1575955) at the start of stimulation. Positive control samples were stimulated using Staphylococcal Enterotoxin B (SEB, List Biological Laboratories) at 1 µg/mL. CD107a BV650 (clone H4A3, BioLegend, Cat. #328643, RRID:AB_2565967) was added at the start of stimulation. Brefeldin A (1 µg /mL) (Sigma-Aldrich) and monensin (0.66 µL/mL) (BD Biosciences) were added one hour after initiation of stimulation. Cells were incubated under stimulation conditions for a total of 9 hours.

All following incubations were performed at room temperature. Cells were stained for viability exclusion using Live/Dead Fixable Aqua for 10 minutes, followed by a 20-minute incubation with a panel of directly conjugated monoclonal antibodies diluted in equal parts of fluorescence-activated cell sorting (FACS) buffer (PBS containing 0.1% sodium azide and 1% bovine serum albumin) and Brilliant stain buffer (BD Biosciences). Fluorophore-conjugated recombinant RBD protein produced by the Hensley Lab (University of Pennsylvania) was used to identify RBD-binding B cells during the surface antibody stain. Cells were washed in FACS buffer and fixed/permeabilized using the FoxP3 Transcription Factor Buffer Kit (eBioscience), following manufacturer’s instructions. Intracellular staining was performed by adding the antibody cocktail prepared in 1X permwash buffer for 1 hour. Stained cells were washed and fixed in PBS containing 1% paraformaldehyde (Sigma-Aldrich) and stored at 4° C. in the dark until acquisition. All flow cytometry data were collected on a BD LSR II or BD FACSymphony A5 cytometer (BD Biosciences). Data were analyzed using FlowJo software (versions 9.9.6 and 10.6.2, Tree Star).

The following antibodies were used: PD1 BV421 (clone EH12.2H7, BioLegend, Cat. #329919, RRID:AB_10900818), CD14 BV510 (clone M5E2, BioLegend, Cat. #301842, RRID:AB_2561946) and APC-Cy7 (clone M5E2, BioLegend, Cat. #301819, RRID:AB_493694), CD16 BV510 (clone 3G8, BioLegend, Cat. #302048, RRID:AB_2562085) and APC-Cy7 (clone 3G8, BioLegend, Cat. #302017, RRID:AB_314217), CD20 BV510 (clone 2H7, BioLegend, Cat. #302339, RRID:AB_2561721) and BV650 (clone 2H7, BioLegend, Cat. #302335, RRID:AB_11218609), CD69 BV605 (clone FN50, BioLegend, Cat. #310937, RRID:AB_2562306), CD21 PECy7 (clone Bu32, BioLegend, Cat. #354911, RRID:AB_2561576), CD4 BUV661 (clone SK3, BD Biosciences, Cat. #612962, RRID:AB_2870238), CD95 BUV737 (clone DX2, BD Biosciences, Cat. #612790, RRID:AB_2870117), CD8 BUV563 (clone RPA-T8, BD Biosciences, Cat. #612914, RRID:AB_2870199), KI67 BV786 (clone B56, BD Biosciences, Cat. #563756, RRID:AB_2732007), IL2 PE (clone MQ1-17H12, BD Biosciences, Cat. #554566, RRID:AB_395483), IFN-gamma BV750 (clone B27, BD Biosciences, Cat. #566357, RRID:AB_2739707), CD3 BUV805 (clone SP34-2, BD Biosciences, Cat. #742053, RRID:AB_2871342), Granzyme B AF700 (clone GB11, BD Biosciences, Cat. #560213, RRID:AB_1645453), CD3 APC-Cy7 (clone SP34-2, BD Biosciences, Cat. #557757, RRID:AB_396863), IgM PECy5 (clone G20-127, BD Biosciences, Cat. #551079, RRID:AB_394036), CD27 BV421 (clone M-T271, BD Biosciences, Cat. #562513, RRID:AB_11153497), HLA-DR BV605 (clone G46-6, BD Biosciences, Cat. #562844, RRID:AB_2744478), CD80 BV786 (clone L307.4, BD Biosciences, Cat. #564159, RRID:AB_2738631), CXCR3 AF488 (clone 1C6, BD Biosciences, Cat. #558047, RRID:AB_397008), CXCR5 SB702 (clone MU5BEE, Thermo Fisher Scientific, Cat. #67-9185-42, RRID:AB_2717183), Tbet PerCP-Cy5.5 (clone 4B10, Thermo Fisher Scientific, Cat. #45-5825-82, RRID:AB_953657), CD11c PECy5.5 (clone 3.9, Thermo Fisher Scientific, Cat. #35-0116-42, RRID:AB_11218511), TNF-alpha PE-Cy7 (clone Mab11, Thermo Fisher Scientific, Cat. #25-7349-41, RRID:AB_1257208), and polyclonal anti-IgD PE Tx Red (SouthernBiotech, Cat. #2030-09, RRID:AB_2795630).

First, to ensure that only live single cells were analyzed from PBMCs, forward scatter height (FSC-H)-versus-forward scatter area (FSC-A) and side scatter area (SSC-A)-versus-FSC-A plots were used to exclude doublets and focus on singlet small lymphocytes. Dead cells were excluded by gating on cells negative for the viability marker Aqua Blue. For T cell function analysis, monocytes, B cells and NK cells were excluded via the CD14/19/16 dump gate. CD4⁺ and CD8⁺ T lymphocytes were gated within CD3⁺ cells. To determine the memory phenotype, CD28 versus CD95 were used, and naive T cells were excluded from the analysis.

For B cell analysis, B cells were identified as CD20⁺ and CD3/CD14/CD16⁻. Memory B cells were defined as CD27⁺ or CD27⁻IgD⁻.

### Example 19—AAV Neutralizing Antibody (NAb) Assay

NAb responses against AAV1, AAV2, AAV5, AAV8, AAV9 and AAVrh32.33 capsids were measured in serum using an in vitro HEK293 cell-based assay and LacZ expressing vectors (Vector Core Laboratory, University of Pennsylvania, Philadelphia, PA) as previously described (Calcedo et al., 2018, Hum. Gene Ther. Methods, 29:86-95). The NAb titer values are reported as the reciprocal of the highest serum dilution at which AAV transduction is reduced 50% compared to the negative control. The limit of detection of the assay was a 1:5 serum dilution.

### Example 20—Biodistribution/Gene Expression Studies

Tissue collection was segregated for genomic DNA (gDNA) or total RNA work by QIASymphony nucleic acid extraction with the aim of filling up 96-well plates of purified material. A small cut of frozen tissue (~20 mg) was used for all extractions with the exception of gDNA purifications from spleen (1-2 mg). Tissues were disrupted and homogenized in QIAGEN Buffer ATL (180 µL) and lysed overnight at 56° C. in the presence of QIAGEN Proteinase K (400 µg) for gDNA, or directly in QIAGEN® Buffer RLT-Plus in the presence of 2-mercaptoethanol and a QIAGEN anti-foaming agent for total RNA purification. Tissue lysates for gDNA extraction were treated in advance with QIAGEN RNase A (400 µg), while tissue homogenates for RNA extraction were DNase-I treated in situ in the QIASymphony® during the procedure. Nucleic acids were quantified only if necessary, as a troubleshooting measure. Purified gDNA samples were diluted 10-fold and in parallel into Cutsmart-buffered BamHI-HF (New England Biolabs) restriction digestions in the presence of 0.1% Pluronic F-68 (50 µL final volume) that ran overnight prior to quantification. Similarly, DNase-I-treated total RNAs were diluted 10-fold into cDNA synthesis reactions (20 µL final volume) with or without reverse transcriptase using the High Capacity cDNA Reverse Transcription Kit (Thermo Fisher™). For ddPCR (gDNA or cDNA) or qPCR (cDNA), 2 µL of processed nucleic acids were used for quantification using Bio-Rad™ or Applied Biosystems™ reagents, respectively, in 20 µL reactions using default amplification parameters without an UNG incubation step. All the studies included negative control (PBS) groups for comparison. The significantly small variance of multiple technical replicates in ddPCR justified the use of a single technical replicate per sample and no less than three biological replicates per group, gender, or time point. coRBD signal for ddPCR and vector biodistribution (gDNA) was multiplexed and normalized against the mouse transferrin receptor (Tfrc) gene TaqMan™ assay using a commercial preparation validated for copy number variation analysis (Thermo Fisher Scientific). Likewise, coRBD signal for ddPCR and gene expression analysis was multiplexed and normalized against the mouse GAPDH gene, also using a commercial preparation of the reference assay (Thermo Fisher Scientific). Target and reference oligonucleotide probes are tagged with different fluorophores at the 5′-end, which allows efficient signal stratification. For qPCR, coRBD and mGAPDH TaqMan assays were run separately to minimize competitive PCR multiplexing issues prior to analysis and delta delta Ct normalization. The limit of detection of the assay was 10 copies/reaction, therefore, wells with less than 10 copies were considered negative.

### Example 21—Phylogenetic Analysis

First, fourteen representative AAV capsid sequences were aligned by Clustal Omega (Sievers and Higgins, 2018, Protein Sci., 27:135-45). Substitution models and model parameters were statistically compared (120 in total) through ProtTest3 (Darriba et al., 2011, Bioinformatics, 27:1164-5), and the Le, Gascuel Model (Le and Gascuel, 2008, Mol. Biol., Evol., 25:1307-20) was selected based on the Aikake Information Criterion (AIC). Additionally, amino acid frequencies were determined empirically through the alignment (+F parameter) and evolutionary rates among sites were allowed to vary within five categories by modeling variability with a discrete Gamma distribution (+G parameter), again selected through AIC. A maximum-likelihood approach was then used to infer the evolutionary relationships among the included sequences using MEGA X (Kumar et al., 2018, Mol. Biol. Evol., 35:1547-49) and the resultant phylogeny rooted along the midpoint of the branch between AAV4 and AAV5 for purposes of visualization. A sequence identity matrix was computed, and the resultant table was used to annotate the phylogeny by percent identity.

### Example 22—Graphs and Statistical Analysis

GraphPad Prism 8 was used for graph preparation and statistical analysis. Data were represented as mean ± standard deviation (SD). Groups were compared between them by One-way ANOVA and Tukey’s tests in studies with more than two groups and n≥10, and Kruskal Wallis and Dunn’s testes were used if n<10. Two groups were compared between them using Student’s t test (if n≥10) or Mann Whitney’s U (if n<10). Pearson’s correlation coefficient was calculated to assess correlation.

### Example 23—Design and Production of AAVCOVID Vaccines

AC1 and AC3 are both viral vector COVID-19 vaccine candidates composed of an AAVrh32.33 capsid and an AAV2 ITR-flanked transgene expressing distinct SARS-CoV-2 S antigens. FIG. 9A depicts AC1 which encodes a full-length membrane anchored S protein based on the Wuhan sequence, modified by amino-acid substitutions that prevent S1/S2 furin cleavage and stabilize S in a pre-fusion conformation for optimal RBD exposure and antigenicity. AC3 expresses the secreted S1 subunit of the Wuhan S protein (FIG. 9A). AAVrh32.33 is a previously described rhesus derived AAV serotype. It is most closely related to AAV4 but phylogenetically divergent from the AAVs that are most commonly circulating and used as gene therapy vectors in humans (FIG. 9B). Previously, it was shown in an extensive human epidemiological study that the seroprevalence of antibodies to AAVrh32.33 is minimal. Consistent with these findings, 50 plasma samples collected from healthy donors demonstrated highly reduced antibody prevalence to AAVrh32.33 as compared with that seen to AAV8 and AAV2, with 6% of samples with titers of 1:20 or above compared to 22% and 28% respectively (FIG. 9C). In terms of production yields, in more than 10 research grade preparations, AC1 was shown to be comparable to serotypes AAV8 and AAV9, while AC3 showed slightly reduced productivity (FIG. 9D). The capsid identity of AC1 and AC3 is consistent with AAVrh32.33 in the AAV-ID thermostability assay (FIG. 9E).

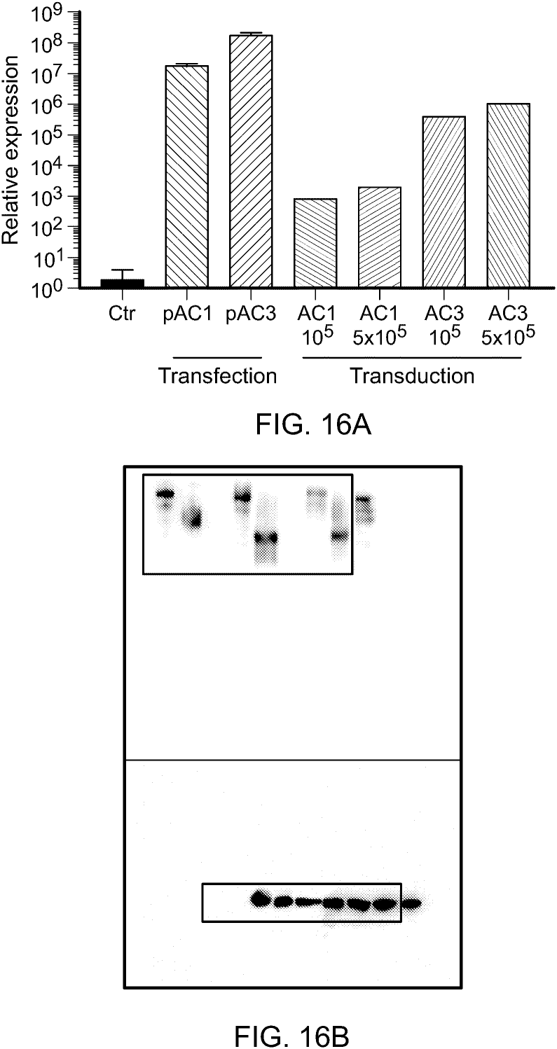

Lastly, expression of the S transgene was detected for each AAVCOVID candidate in vitro by transfection and transduction (FIGS. 9F and 16). Higher expression of AC3 was detected at mRNA (FIG. 16A) and protein level (FIGS. 9F and 16).

### Example 24—A Single Dose of AAVCOVID Induces High and Durable Antibody Titer in Two Mouse Strains

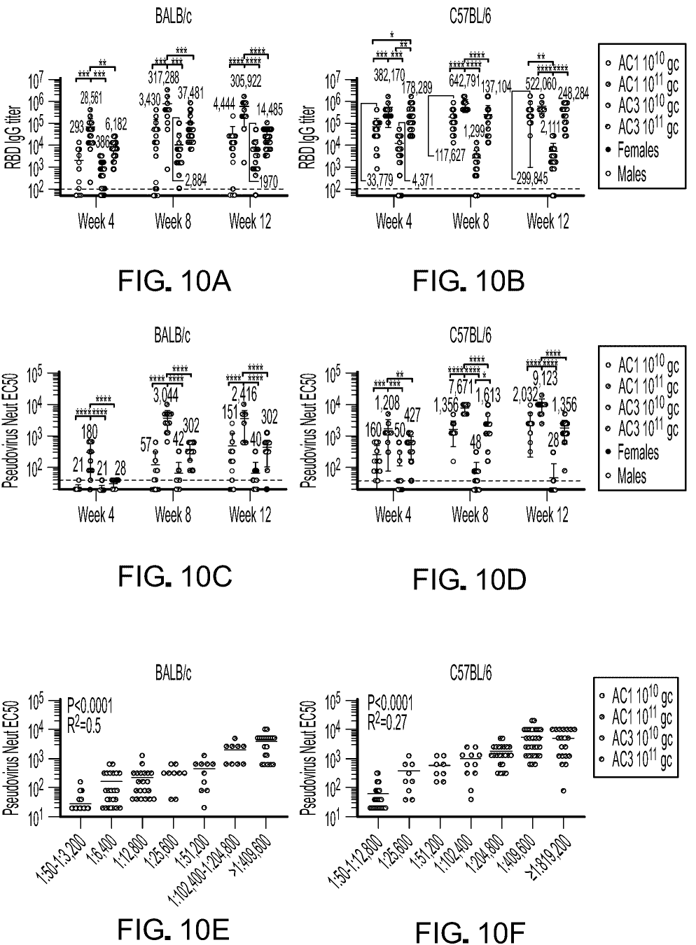

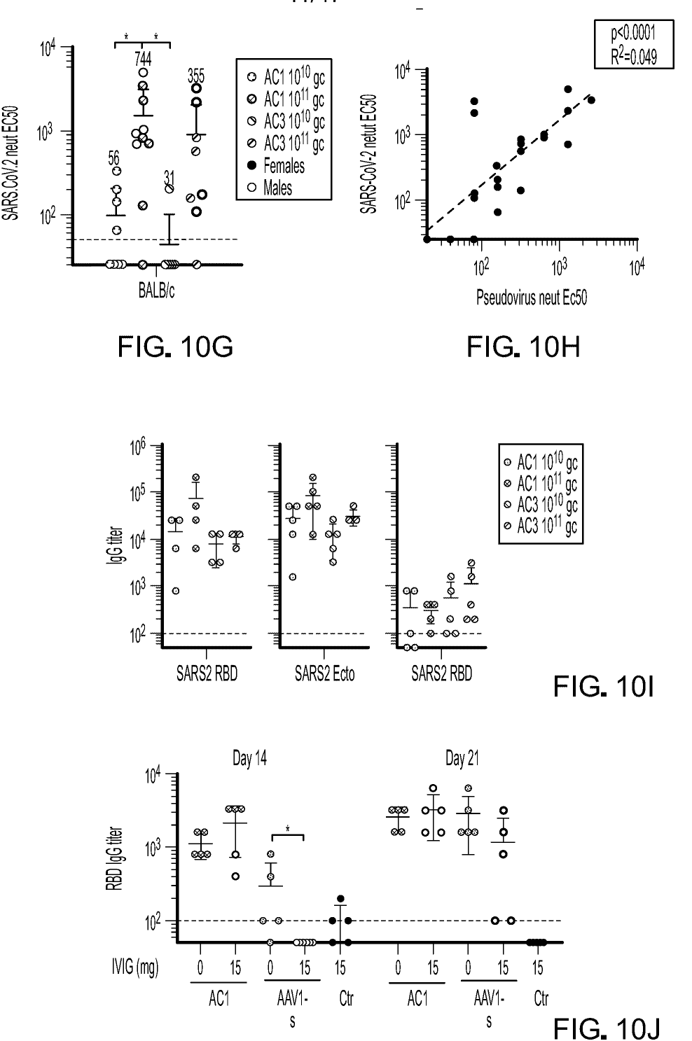

The immunogenicity of AC1 and AC3 following a single injection at a low and high dose of 10e10 and 10e11 gc, respectively, in the gastrocnemius muscle was evaluated in 6-10-week-old BALB/C and C57BL/6 mice of both genders. SARS-CoV-2 (SARS2) RBD-binding IgG antibody levels were monitored by ELISA at regular intervals (FIGS. 10A and 10B), as were neutralizing antibody levels assayed using a SARS-CoV-2 Spike pseudotyped lentivirus (pseudovirus) inhibition-of-transduction method (FIGS. 10C and 10D).

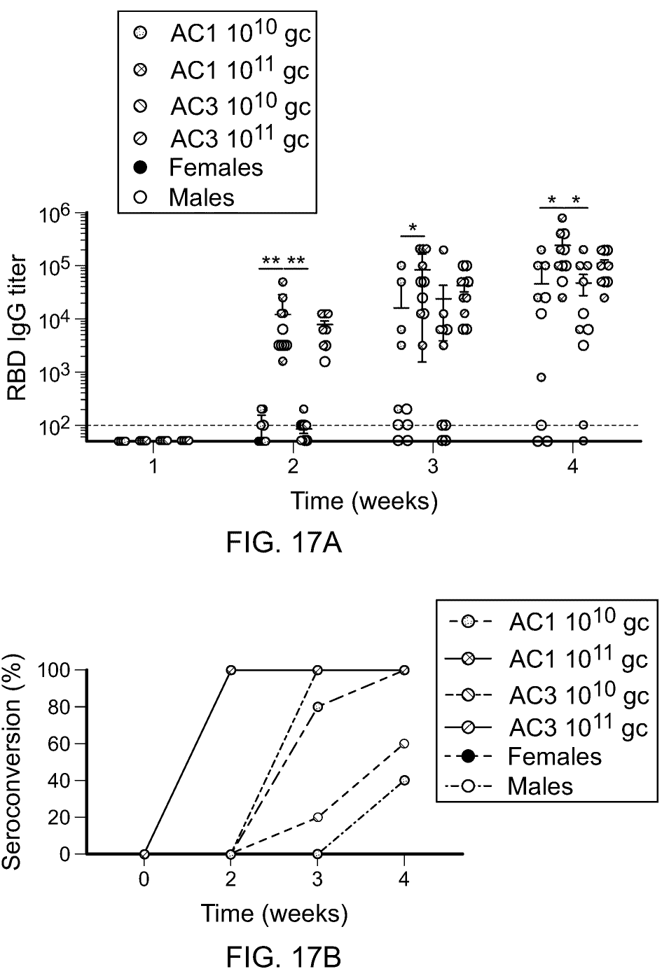

Both mouse strains demonstrated dose-dependent potent binding and neutralizing responses from a single dose administration of AC1 or AC3 that persisted through 3 months. Overall, AC1 at high doses induced a significantly higher level of binding and neutralizing antibody titers to SARS-CoV-2 (binding geometric mean titer (GMT) of 305,922 and 522,060 in BALB/c and C57BL/6, respectively; and neutralizing GMT of 2,416 and 9,123, 12 weeks post-vaccination) than AC3 (binding GMT of 14,485 and 248,284 in BALB/c and C57BL/6, respectively; and neutralizing GMT of 302 and 1,356, 12 weeks post-vaccination). At a low dose, AC1 was superior to AC3, particularly in C57BL/6 mice at later timepoints (FIGS. 10A-10F). Immunogenicity was modestly lower in males versus female mice for both candidates (FIGS. 10A-10D). The kinetics of binding-antibody induction showed early onset of responses by day 14 (FIG. 17A) and increasing seroconversion rates over time (FIG. 17B). Neutralizing antibody kinetics lagged by approximately a week, with limited seroconversion at week 4 that increased thereafter (FIGS. 10C and 10D). Binding and neutralizing titers correlated; however, AC1 achieved higher neutralizing titers and a larger relative ratio of neutralizing to binding titers compared to those produced by AC3 (FIGS. 10E and 10F).

Limited plaque reduction neutralizing assay titers (PRNT) with live SARS-CoV-2 were obtained for AC1 and AC3 in BALB/c mice 4 weeks after vaccination, showing the quality of response in terms of the neutralization of SARS-CoV-2 live virus (FIG. 10G). These responses correlated modestly well with results from the pseudovirus neutralization assay (FIG. 10H). ELISA IgG titers to SARS-CoV-2 S full-length ectodomain (SARS2 Ecto), SARS-CoV-1 S RBD (SARS RBD) or MERS S RBD were assayed (FIG. 10I). Antibody responses to full length S ectodomain (SARS2 Ecto) were modestly higher compared to RBD titers (SARS2 RBD) (FIG. 10I). Cross-reactivity of the elicited IgG with SARS RBD was noted, but at reduced levels (FIG. 10I), with no cross-reactivity detected against MERS RBD.

Lastly, to model the impact of AAV capsid pre-existing immunity on AAVCOVID immunogenicity in humans, 24 and 2 hours before vaccination BALB/c mice received 15 mg of intravenous immunoglobulin (IVIG) derived from pooled samples from thousands of human donors. As a control, a single dose immunization using the AC1 vector was compared to vaccination with an AAV1 capsid vector containing an identical genome (AAV1-S). AAV1 is known to have higher pre-existing immunity in human populations. FIG. 10J shows that animals vaccinated with AC1 were unaffected by the IVIG pretreatment, while AAV1-S had reduced seroconversion on day 21 compared to IVIG-naive animals.

For FIGS. 10A-10J, data are represented as mean ± SD. For FIGS. 10A-10D and 10G, groups were compared by one-way ANOVA and Tukey’s post-test. For FIGS. 10E, 10F, and 10H, Pearson’s correlation coefficient was calculated to assess correlation. For FIG. 10J, naive and immunized groups were compared by Mann-Whitney’s U test.

### Example 25—AC1 Elicits Qualitatively Distinct Humoral Response Compared to AC3

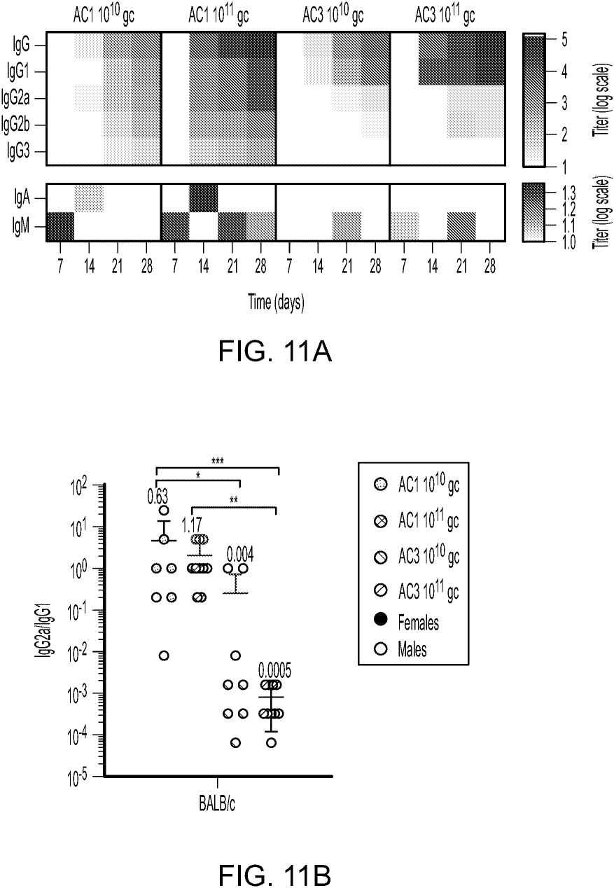

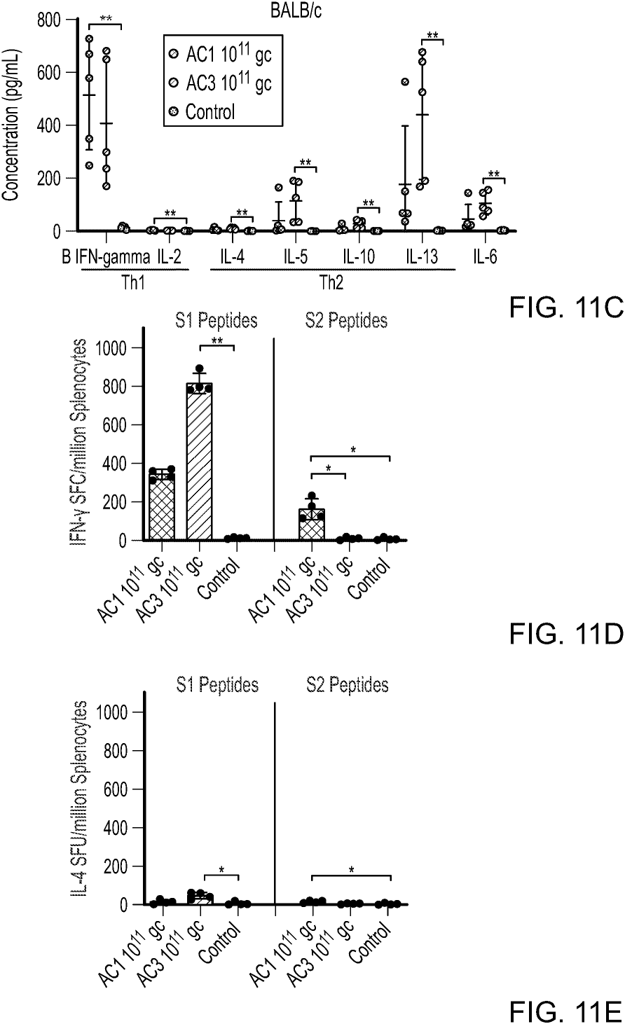

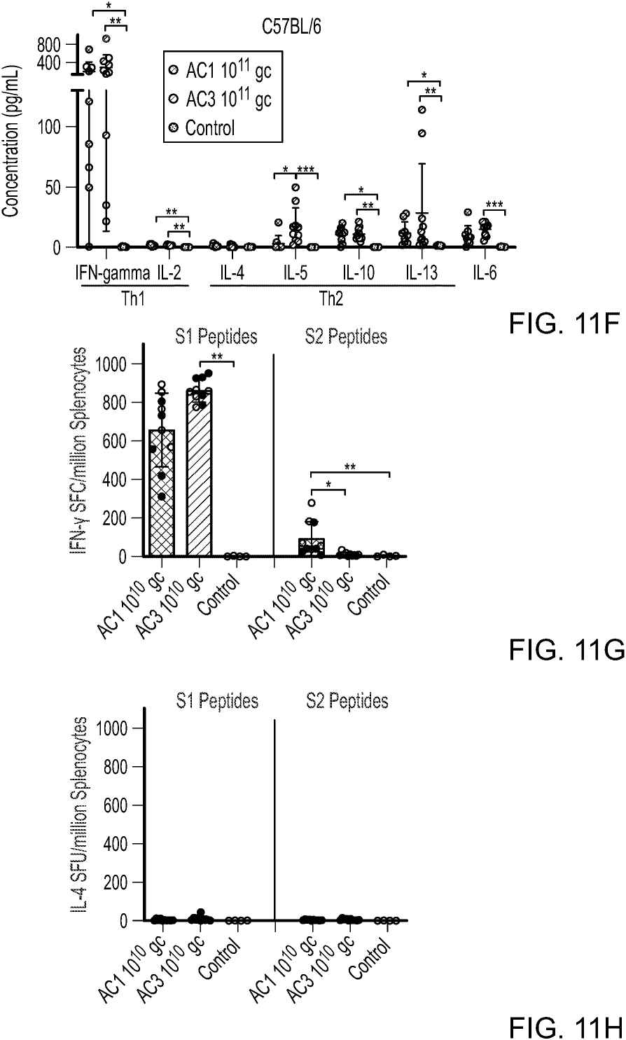

Next, the quality of the humoral responses was assessed in BALB/c mice over time for each of the vaccine candidates. In AC1-treated animals, IgM and IgA antibodies directed at SARS2 RBD were detected at early timepoints, day 7 and 14, respectively, but the IgG isotype dominated circulating SARS2 RBD-specific antibody levels from thereon (FIG. 11A). AC3 IgM and IgA levels were lower than those observed for AC1. Total IgG levels were composed of all IgG subclasses when AC1 was utilized, whereas IgG1 predominated when AC3 was injected. The ratio of IgG2a/IgG1 suggests a balanced Th1 response stimulated by AC1, with more Th2 skewing seen in the AC3 response (FIGS. 11A and 11B).

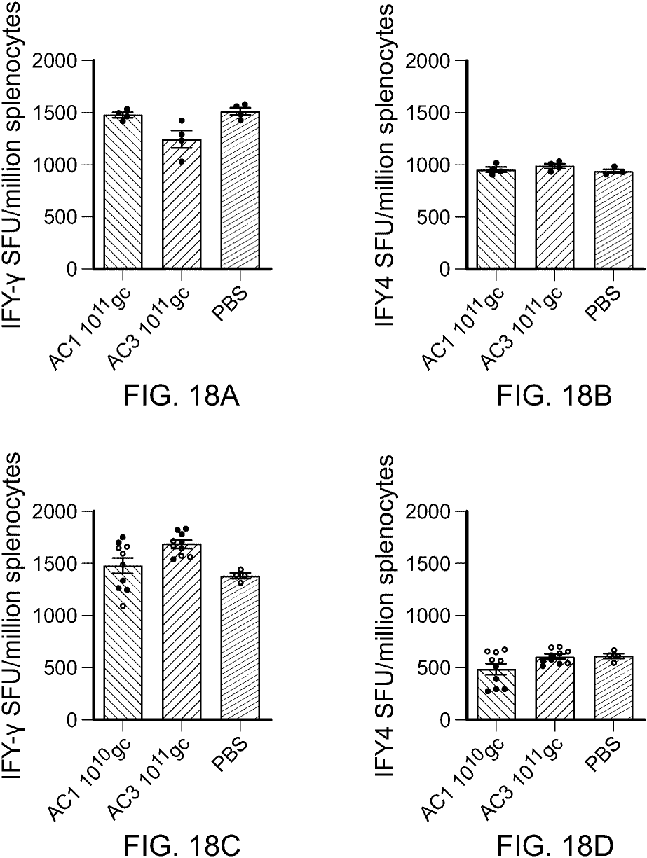

To further interrogate this divergent qualitative response, cytokine secretion and ELISPOT analyses were performed on splenocytes from AC1 and AC3 immunized BALB/c and C57BL/6 animals. Secretion of several cytokines was detected in stimulated splenocytes (FIGS. 11C and 11F). However, IFN-gamma was predominantly secreted and minimal levels of Th2-associated cytokines, such as IL-5 and IL-13, were measured, except in BALB/c mice, where AC3 induced a greater IL-13 response (FIGS. 11C and 11F). IFN-gamma ELISPOT revealed a robust response against peptides spanning the S1 subunit (FIGS. 11D, 11G, 18A and 18C), while lower responses were detected against the S2 subunit only in the AC1 vaccinated group. Minimal IL4 responses were seen by IL4 ELISPOT (FIGS. 11E, 11H, 18B and 18D).

For FIGS. 11B-11H, the data are represented as mean ± SD and groups were compared by Kruskal Wallis and Dunn’s post-test.

### Example 26—Immunogenicity of AAVCOVID Is Influenced by Age but Retains Potency in Obese Mice

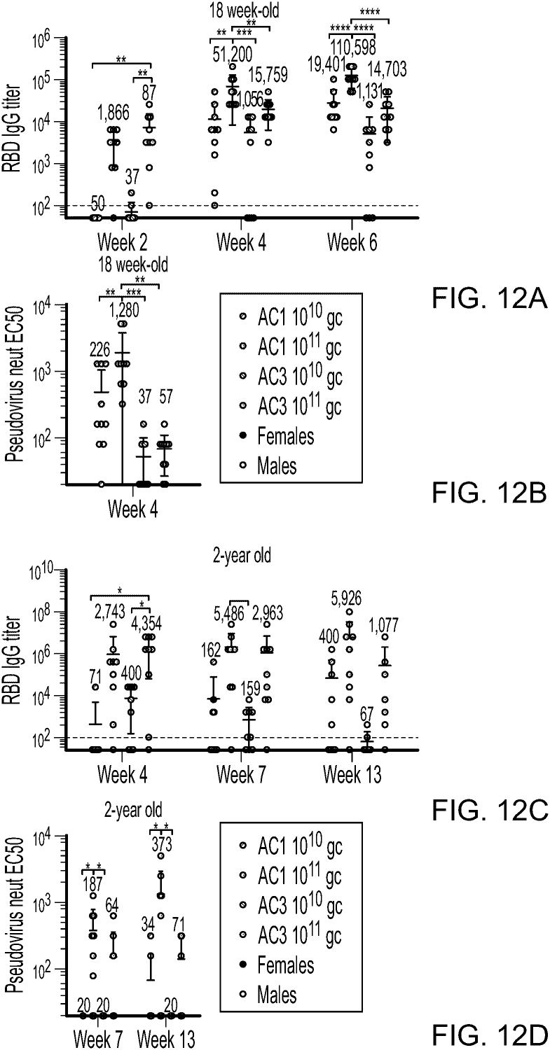

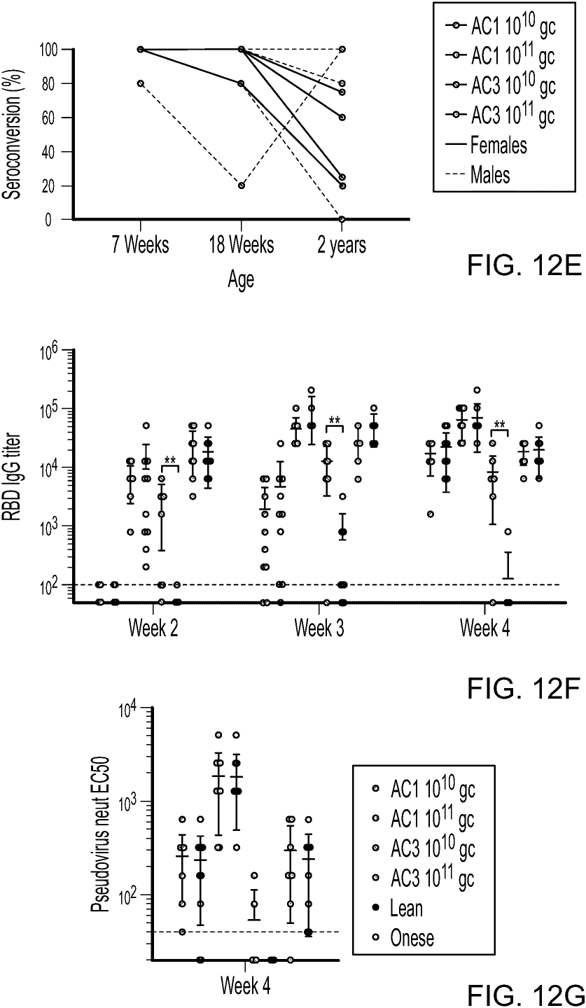

Vaccine efficacy is often impaired in obese or elderly humans, which are two of the most vulnerable populations in the COVID-19 pandemic. To model this conditions, 18-week and 2-year-old mice of both genders were immunized with AAVCOVID at low and high doses, bled at regular intervals, and analyzed for SARS2 RBD IgG and pseudovirus neutralization responses in the serum. A reduction in IgG and neutralizing titers is observed between 18-week and 2-year-old mice (FIGS. 12A-12D). 18-week-old and, to a lesser extent, 2-year-old mice developed robust neutralizing titers upon vaccination with AC1 (FIGS. 12B and 12D), but the AC3 at high doses failed to recapitulate the results in younger mice (FIG. 12D). Low doses and high dose of AC3 failed to elicit neutralizing antibodies in most of the 2-year-old mice, while animals treated with a high dose of AC1 showed high titers but incomplete seroconversion (FIGS. 12D and 12E). In aggregate, high dose AC1 demonstrates robust, albeit reduced immunogenicity in aged mice, with clearly superior immunogenicity compared to that induced by AC3 in aged mice.

A diet-induced C57BL/6 obesity (DIO) mouse model was used to study vaccine efficacy in inducing SARS2 RBD-specific antibodies in overweight animals. 12-week-old C57BL/6 and C57BL/6 DIO (n = 10) mice were vaccinated with 10¹⁰ and 10¹¹ gc of AC1 and AC3. IgG RBD-binding and neutralizing antibody levels were indistinguishable between lean and obese groups for AC1 and the high dose group of AC3, yet interestingly, the low dose of AC3 produced a less robust antibody response in the DIO mice than did the comparable dose of AC1 (FIGS. 12F and 12G).

In FIGS. 12A-12D and 12F-12G, data are represented as mean ± SD. For FIGS. 12A-12D, groups were compared by one-way ANOVA and Tukey’s post-test. FIGS. 12F-12G show lean and obese mice receiving the same treatment were compared by Student’s t test.

### Example 27—Durable Neutralizing Antigenicity in NHP From a Single Dose Injection

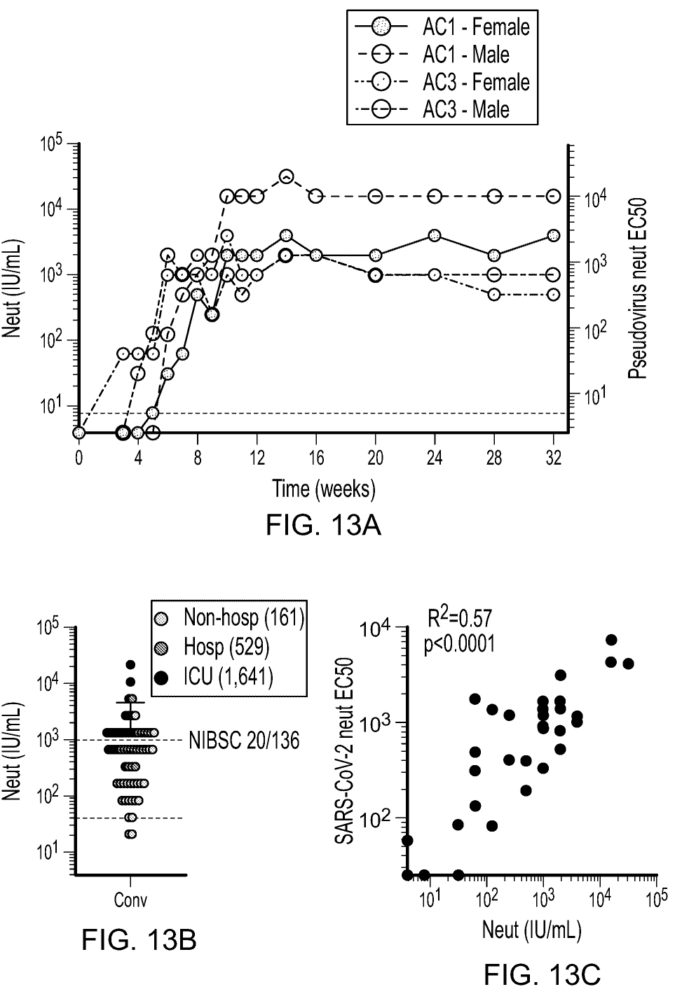

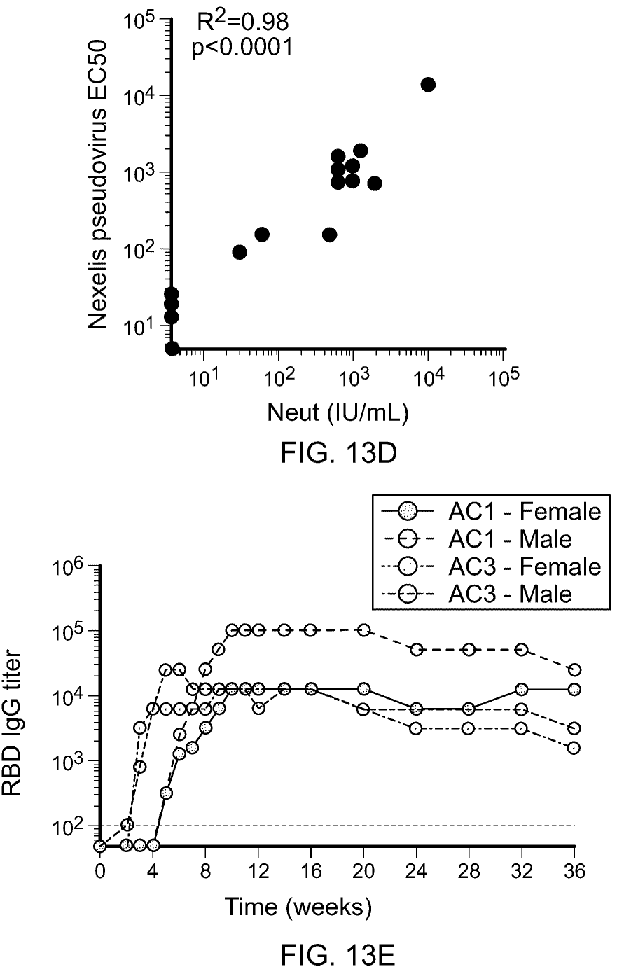

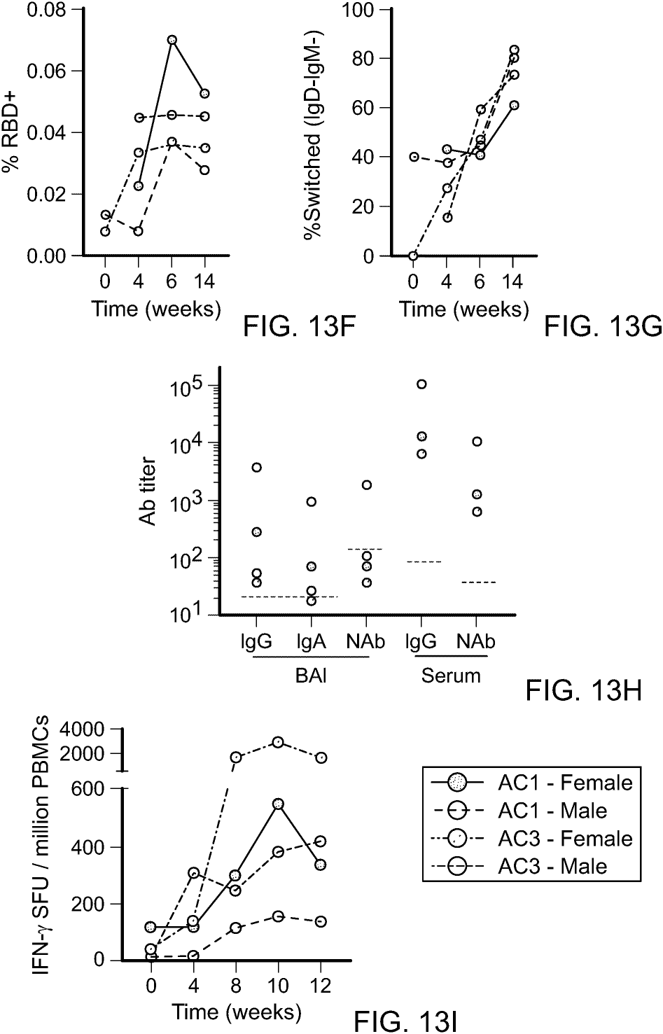

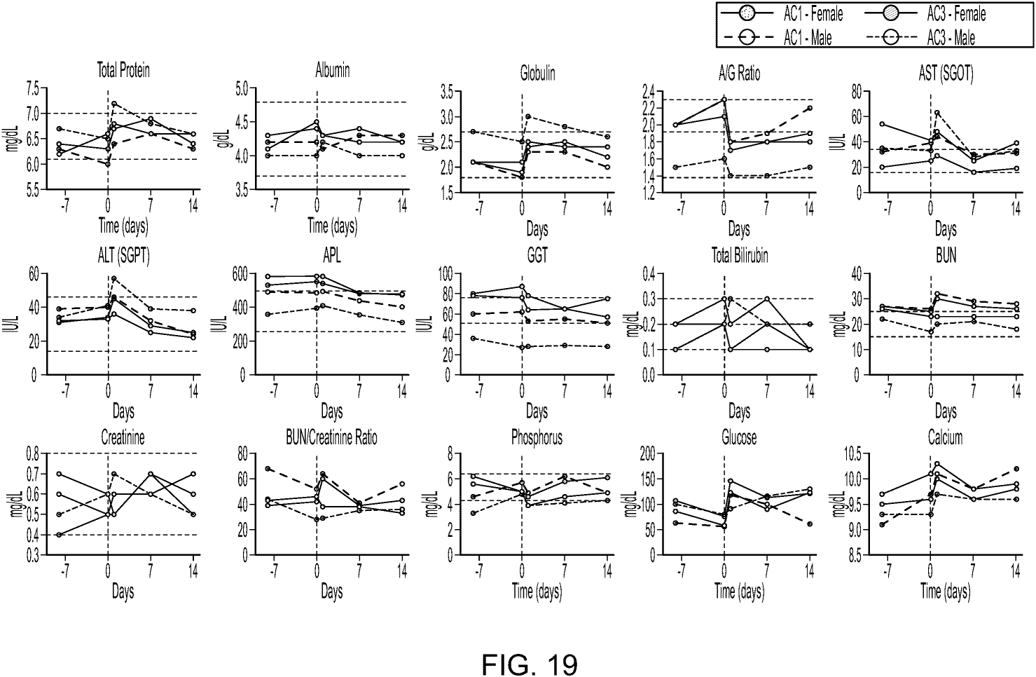

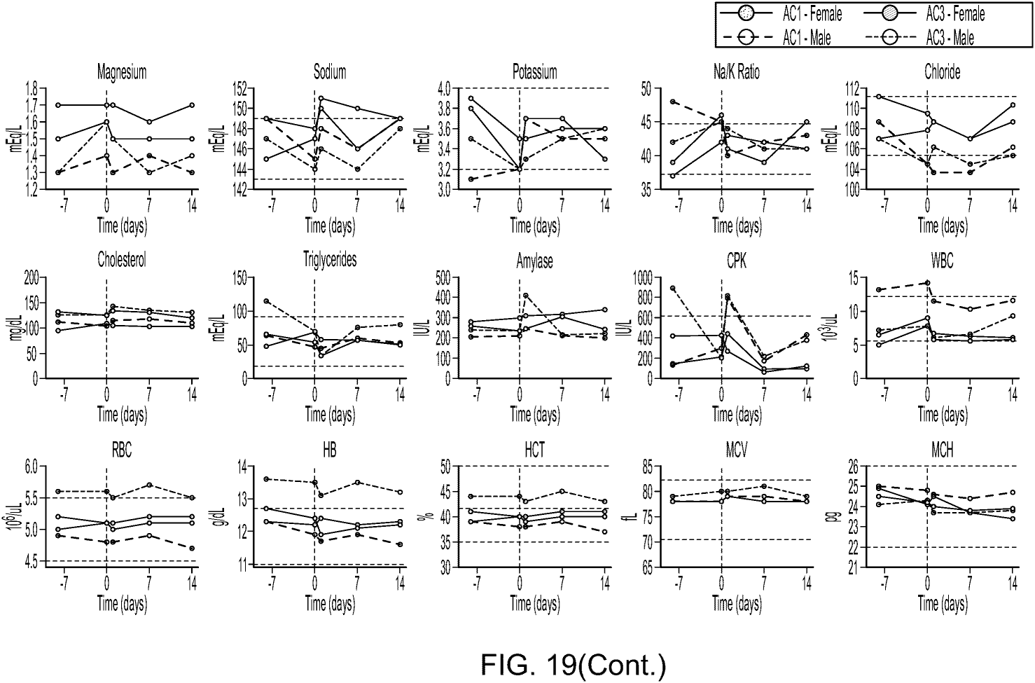

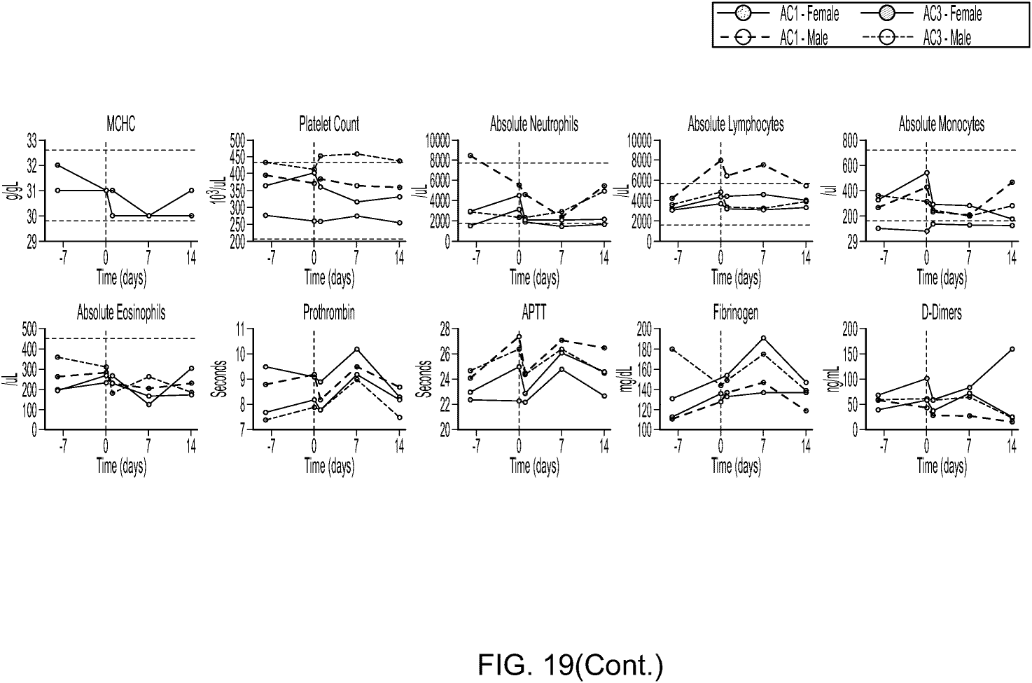

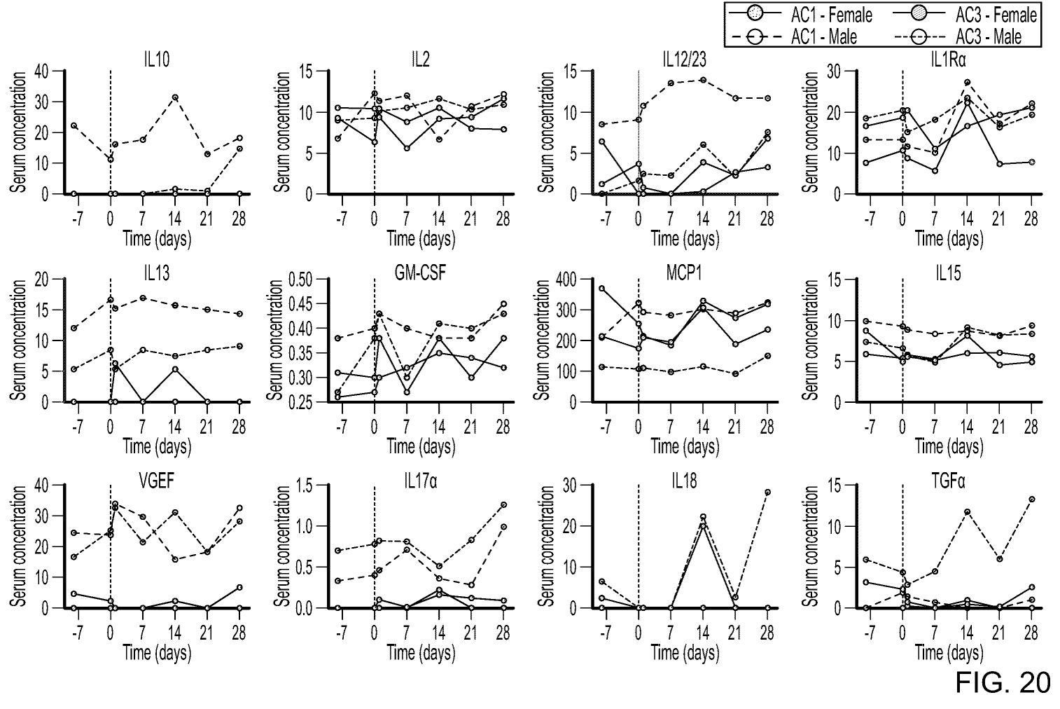

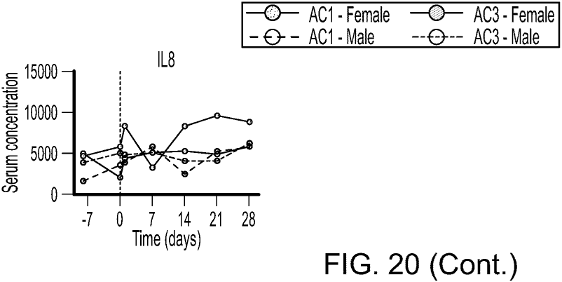

To model the immunogenicity of AAVCOVID in humans, one female and one male rhesus macaque were injected IM with 10¹² gc of AC1 and AC3. Animals tolerated the vaccine dose well, with no temperature elevations or local reactogenicity based on clinical examinations, complete blood counts and chemistry (FIG. 19), or cytokine analysis (FIG. 19). Regular phlebotomies were performed to assess RBD binding, pseudovirus neutralizing, and live SARS-CoV-2 neutralizing antibody titers in serum (FIGS. 13A, 13B and 13C) and B cell analysis from PBMCs (FIGS. 13D, 13E and 13F). These animals continue to be monitored to assess the durability of the vaccine response and are currently at the 5-month time point following the single dose immunization.

AC3 SARS2 RBD-binding antibody responses were detectable as early as week 3 after a single administration and plateaued by week 5 hovering around 1:6,400 and 1: 12,800 (FIG. 13A). AC1 IgG, on the contrary, only became apparent on week 5 and then steadily increased until week 10. One AC1-injected animal achieved similar antibody levels to those measured in both AC3 vaccinated primates (1:12,800) while the other AC-1 vaccinated animal achieved levels that were 8-fold higher (FIG. 13A). With minimal fluctuation, SARS2 RBD IgG levels have been maintained to date at peak levels, now 20 weeks or 5 months after a single shot vaccine for both the AC1 and AC3 injections.

Pseudovirus neutralizing titers and PRNT closely tracked with a slight delay in the IgG kinetics for both AC1 and AC3, reaching peak neutralizing titers 6 to 8 weeks after vaccination for AC3 (1:640 and 1:1,280) and 11 weeks following AC1 injection (1:1,280 and 1:10,240). These neutralizing antibody responses have remained stable at peak levels through week 20 in the pseudovirus neutralizing assay and 16 weeks in the PRNT assay (FIGS. 13B and 13C), the last time points analyzed. Benchmarking of the pseudovirus neutralizing assay was performed in 2 ways. First, 60 human convalescent plasma samples from 3 cohorts were analyzed (FIG. 13B) (non-hospitalized (GMT: 154), hospitalized yet not critical (GMT: 508) and ICU patients (GMT: 1,576)), which demonstrated a clear increase of neutralizing titers with severity of disease. Second, a provisional World Health Organization recommended reference plasma (NISBC 20/130) yielded a 1: 1,280 titer in our assay, which was in line with the reported values (FIG. 13B). In summary, AC1 induces neutralizing titers in the range of 1:1,280 and 1:10,240 which is in the higher range of convalescence of hospitalized and ICU patients while AC3 leads to titers of 1:640-1:1,280 which is in the range of hospitalized non-ICU patients. These titers persist for at least 5 months.

To track vaccine-induced peripheral blood B cells, a double-labeling technique with fluorophore-conjugated SARS2 recombinant RBD protein was utilized (FIG. 13D) (Johnson et al., 2020, Immunity, 52:842-55; Knox et al., 2017, JCI Insight, 2:e92943). RBD-binding memory B cells (MBCs) were absent at week 0 and detectable by week 4 in three of the animals (FIG. 13E). RBD-specific MBCs peaked in frequency at 6 weeks post-vaccination in all recipients and were maintained at a similar level at least through week 14 (FIG. 13E). Surface immunoglobulin isotype analyses found an early bias toward generation of IgM-expressing MBCs, whereas isotype switched (IgD⁻IgM⁻; likely IgG⁺) MBCs dominated the SARS2 RBD-specific response by week 14 (FIG. 13F). These findings suggest durable induction of SARS2 RBD-specific memory B cells by both AC1 and AC3 vaccines.

Interestingly, in AC3 injected primates, the secreted S1 protein was detectable in their serum 2 weeks after injection. However, the S protein returned to undetectable levels in both animals by week 4, concurrent with increasing anti-SARS2 RBD antibody titers (FIG. 13G). Similar to the mouse data, SARS2 ectodomain IgG levels in NHP were higher than SARS2 RBD IgG, and modest cross-reactivity to SARS1 RBD was detected (FIG. 13H). Total IgG for both AC1 and AC3 animals was primarily composed of IgG1, suggestive that, in NHP, as opposed to mice, both responses appear more ThI-like (FIG. 13I).

### Example 28—Memory T Cell Response to Spike Antigen Is Developed in NHP

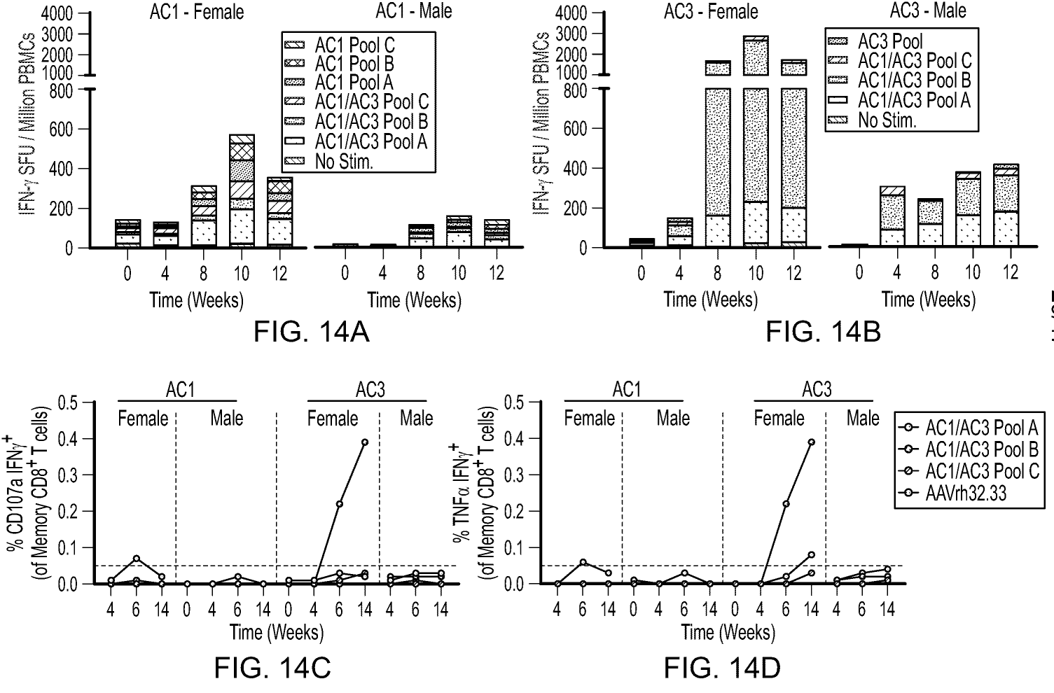

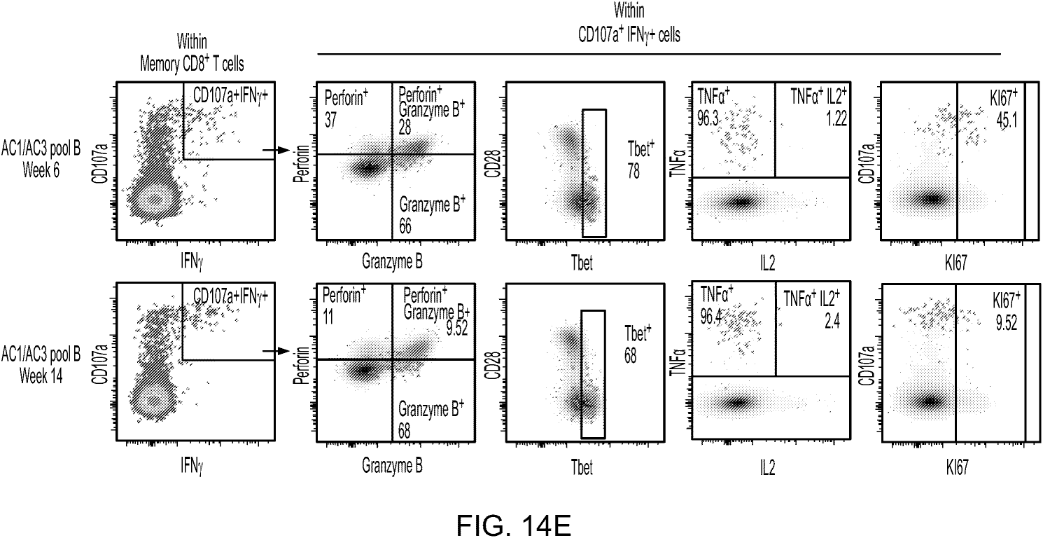

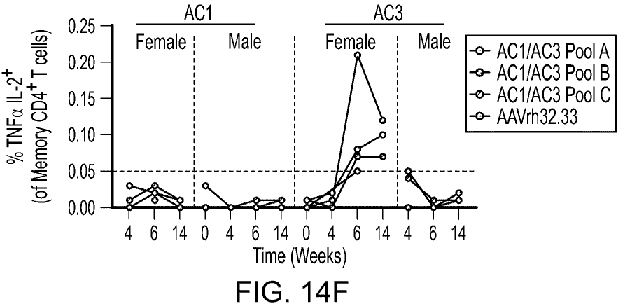

T cell responses to transgene peptide pools (FIG. S6A) were analyzed by IFN-gamma ELISPOT (FIGS. 14A and 14B) and intracellular cytokine staining (ICS) (FIGS. 14C-14F) from PBMCs harvested at monthly intervals. AC3 injected animals showed responses specific to the S1 subunit, higher in the female, starting on week 4 (FIG. 14B); however, lower responses were detected in the AC1 female starting on week 8 and there was only a minimal response in the AC1 vaccinated male (FIG. 14A).

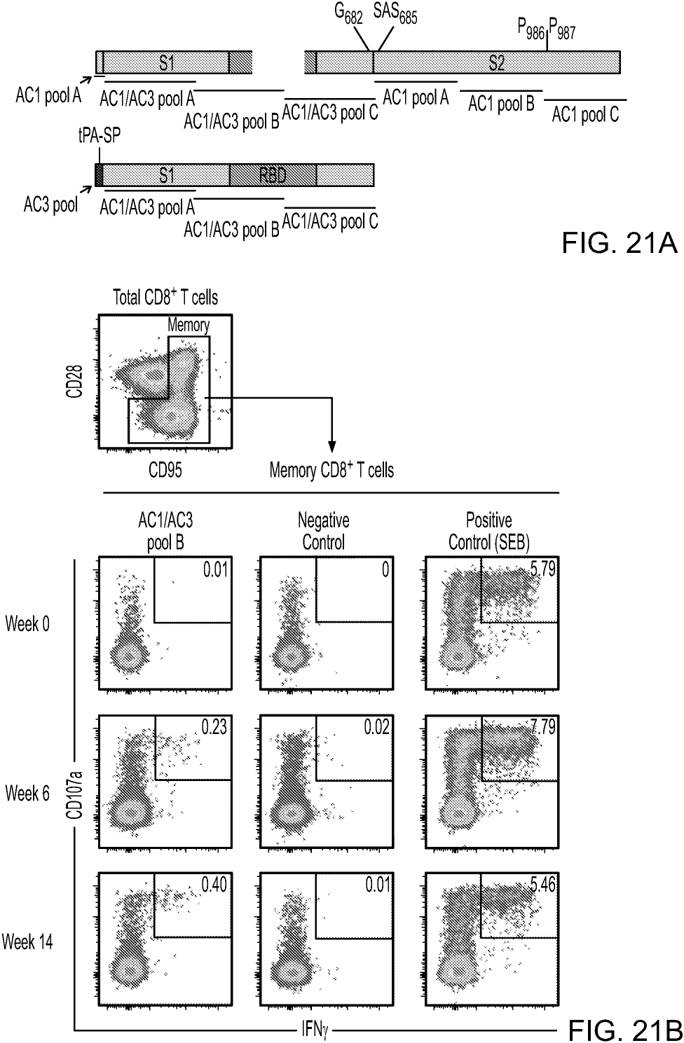

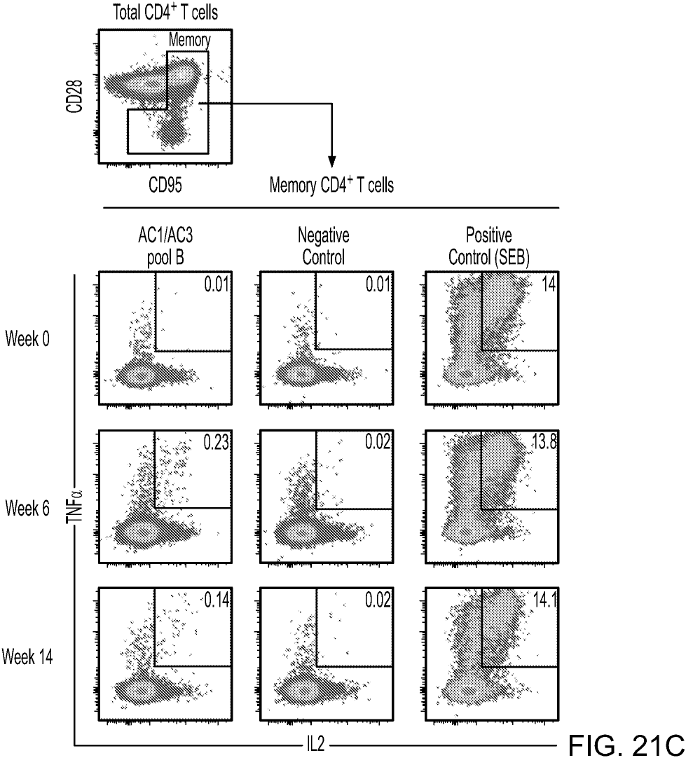

Flow cytometry was used to identify the phenotype and functionality of S-specific cells after stimulating PBMCs with the overlapping S1 peptides (note that S2-specific responses in AC1 animals, which were clearly detected by ELISPOT, were not studied in this analyses). The female AC3 showed a robust memory CD8⁺ T cell response to the S1 subunit beginning at week 6 (FIGS. 14C, 14D and 21B). CD107a⁺ IFN-gamma⁺ responding cells also produced TNFalpha at week 6 and 14 (FIGS. 14D and 14E), but the cytotoxic and activation profile changed over time. At week 6, the S1 subunit-specific cells showed higher expression of the cytotoxicity markers perforin and granzyme B and the activation marker KI67, compared to week 14 (FIG. 14E). S1-specific memory CD4⁺ T cell responses were also detected through production of TNFalpha and IL2 in the female treated with AC3 at week 6 and 14, although these were proportionately lower compared to the corresponding memory CD8⁺ T cell responses (FIGS. 14F and 21C).

### Example 29—NHPs Develop Slow Neutralizing Antibody Response to AAVrh32.33 Capsid That Shows No Cross-Reactivity With Other AAV Serotypes

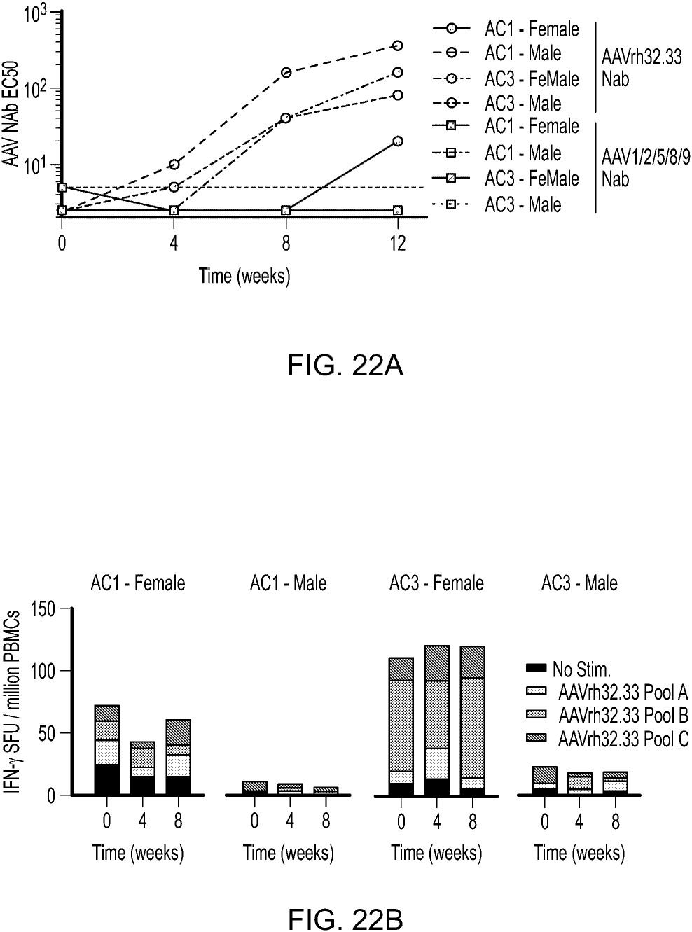

Viral vectored vaccines are known to induce responses to the delivery vector component, in this case, to the AAV capsid. These can enhance the overall immunogenicity of the vaccine, influence its reactogenicity, or prevent the effectiveness of subsequent dosing with a homologous vector due to the neutralization of the vector upon re-administration. Similarly, in the context of AAV, the cross-reactivity of these antibodies may affect subsequent applications of alternative AAV serotypes that could be neutralized via cross-reactive antibodies to AAVrh32.33, thus potentially influencing future applications of gene therapy for subjects vaccinated with AAVCOVID. In this rhesus study, Table S1 shows that AAVrh32.33 neutralizing antibodies did develop, albeit with slow kinetics and to relatively low levels. Importantly, these modest AAV neutralizing responses did not exhibit cross-neutralization of a panel of commonly used AAV gene therapy serotypes AAV1, 2, 5, 8, and 9 (Table 1 and FIG. 22A). In addition, no significant increase in cellular responses against capsid peptides were detected in PBMCs up to 2 months after vaccination (FIG. 22B).

TABLE 1

Neutralizing AAV responses elicited by AAVCOVID in NHP. Neutralizing antibody titers against the injected vector (AAVrh32.33) and cross-reactive neutralizing against other serotypes (AAV1, AAV2, AAV5, AAV8, AAV9) monthly after vaccination of Rhesus macaques

| Animal | Time (weeks) | AAVrh32.33 | AAV1 | AAV2 | AAV5 | AAV8 | AAV9 |
|---|---|---|---|---|---|---|---|
| AC1 -female | 0 | <5 | <5 | <5 | <5 | 5 | <5 |
| | 4 | <5 | <5 | <5 | <5 | <5 | <5 |
| | 8 | <5 | <5 | <5 | <5 | <5 | <5 |
| | 12 | 20 | <5 | <5 | <5 | <5 | <5 |
| | 14/16/20 | 80/80/160 | N/A | N/A | N/A | N/A | N/A |
| AC1 -male | 0 | <5 | <5 | <5 | <5 | <5 | <5 |
| | 4 | 10 | <5 | <5 | <5 | <5 | <5 |
| | 8 | 160 | <5 | <5 | <5 | <5 | <5 |
| | 12 | 320 | <5 | <5 | <5 | <5 | <5 |
| | 14/16/20 | 320/320/320 | N/A | N/A | N/A | N/A | N/A |
| AC3 -female | 0 | <5 | <5 | <5 | <5 | <5 | <5 |
| | 4 | <5 | <5 | <5 | <5 | <5 | <5 |
| | 8 | 40 | <5 | <5 | <5 | <5 | N/A |
| | 12 | 160 | <5 | <5 | <5 | <5 | <5 |
| | 14/16/20 | 160/160/160 | N/A | N/A | N/A | N/A | N/A |
| AC3 -male | 0 | <5 | <5 | <5 | <5 | <5 | <5 |
| | 4 | 5 | <5 | <5 | <5 | <5 | <5 |
| | 8 | 40 | <5 | <5 | <5 | <5 | <5 |
| | 12 | 80 | <5 | <5 | <5 | <5 | <5 |
| | 14/16/20 | 320/320/160 | N/A | N/A | N/A | N/A | N/A |

### Example 30-Vector Is Retained in the Injection Site and Cleared Over Time in Mouse

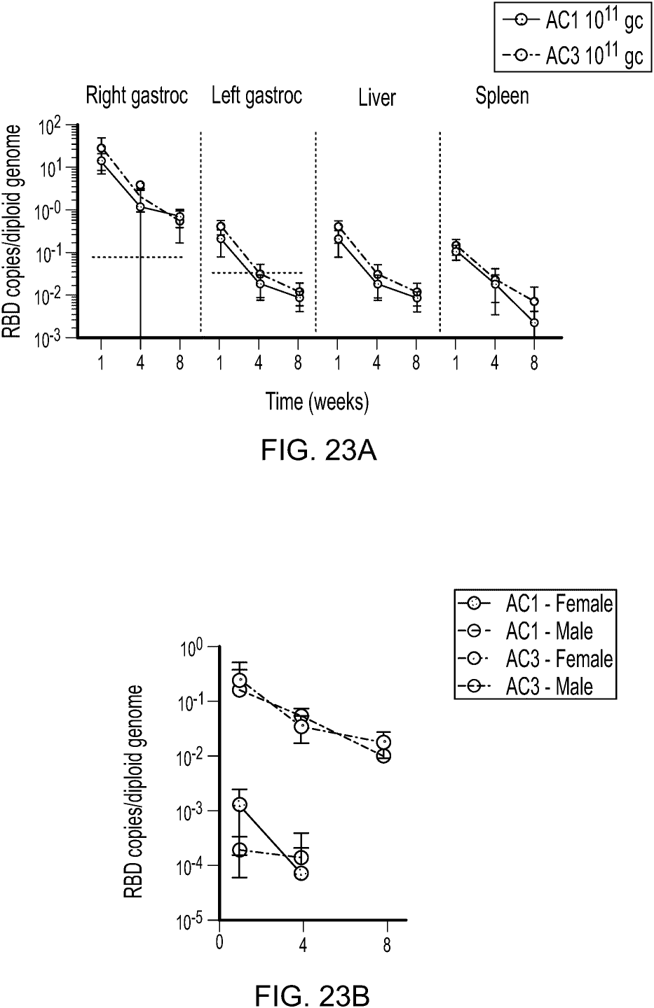

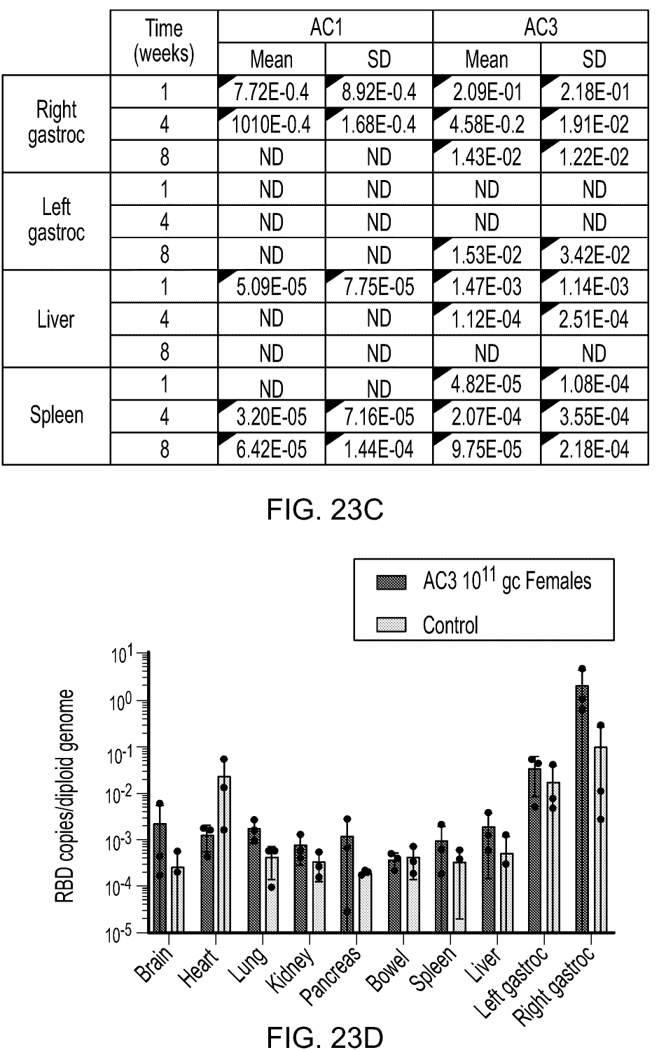

A biodistribution of the vector following AAVCOVID intramuscular injection was analyzed to establish the kinetics of transgene expression and identify which tissues were transduced beyond that of the intended muscle target (FIG. 23). Previously, an AAVrh32.33 expressing a non-self-transgene, when injected intramuscularly in mice, showed declining transgene expression over time that was associated with increasing inflammatory infiltrates at the injection site several weeks after injection. This is in stark contrast to other AAVs expressing the same transgene which led to stable transgene expression and minimal local inflammation. In the current experiment, C57BL/6 mice were injected with 10¹¹ gc in the right gastrocnemius muscle. Animals were euthanized 1, 4 and 8 weeks after vaccination, and tissues were analyzed for vector genome copies and transgene expression. As observed in FIG. 23A, vector genome copies in the injected muscle decreased more than 20-fold from week 1 to week 8. AC3 transgene expression declined in a manner similar to the decline in DNA vector genome copy number. Remarkably, AC1 transgene expression was lower than AC3 expression, close to background levels, possibly due to lower promoter activity (FIG. 23B). Gene transfer and transduction levels of the contralateral gastrocnemius muscle, liver, and spleen demonstrated 10-100-fold less vector DNA at week 1 than measured in the injected muscle with a steady decline of vector DNA and RNA, at times to undetectable levels (FIGS. 23A and 23C). A more comprehensive biodistribution study in BALB/c mice that received the 10¹¹ gc IM dose of AC3 and were euthanized at week 8 further indicated that the predominant tissue of vector genome and transgene expression was the injected muscle (FIG. 23D).

### Example 31—AAVCOVID is Stable and Retains Potency After One-Month Room Temperature Storage

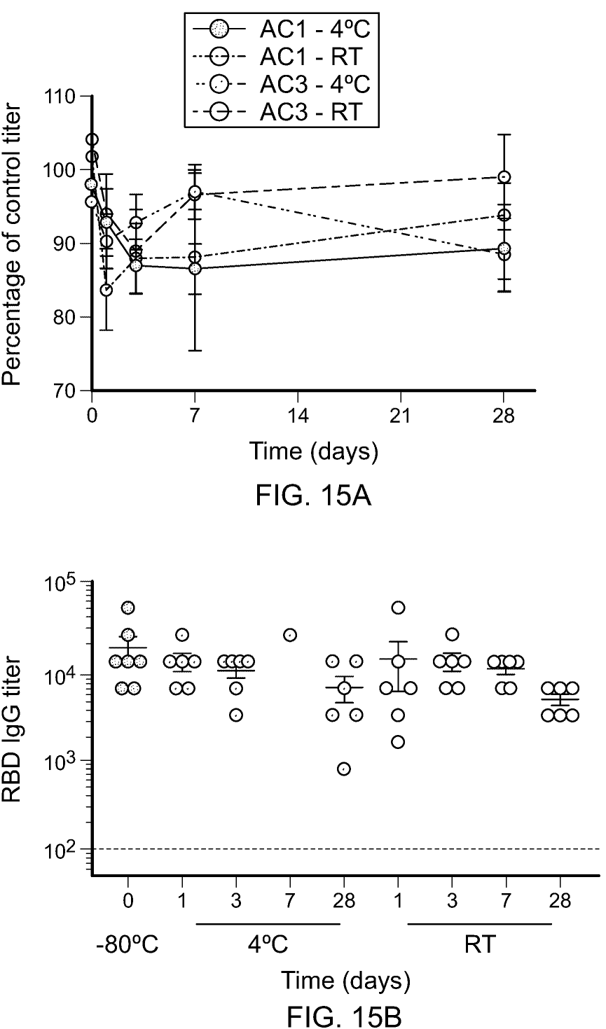

To interrogate the cold chain requirements for storage and transportation of AAVCOVID, research grade vaccine preparations were aliquoted and stored at different temperature conditions (-80° C., 4° C. or room temperature (RT)) for 1, 3, 7 or 28 days. Physical vector stability was assessed by titration of DNAse resistant vector genomes and loss or degradation was assessed by comparison to vector aliquots stored at -80° C. (FIG. 15A and Table 2). After being stored at 4° C. or RT, neither AC1 nor AC3 show a reduction of titers for at least one month. In addition, potency was assessed by injection of 5 × 10¹⁰ gc of AC1 aliquots in female BALB/c mice. Animals vaccinated with AC1 stored at 4° C. or RT for up to 28 days showed similar levels of antibody compared to a control group that received vaccine vectors stored at -80° C. (FIG. 15B). Although not significant, antibody titers trended downwards with time. Larger studies are performed to elucidate if potency can be maintained for longer periods.

TABLE 2

AAVCOVID stability assessment. Titration (gc/mL) and percentage of titer relative to the initial titer of AC1 and AC3 aliquots stored at different temperatures for 1, 3, 7 or 28 days

| | | Titer (gc/mL) | | Relative titer | |
|---|---|---|---|---|---|
| Temp | Time (days) | AC1 | AC3 | AC1 | AC3 |
| -80° C. | NA | 2.65E+12 | 2.47E+12 | 100.00% | 100.00% |
| 4° C. | 1 | 2.46E+12 | 2.23E+12 | 92.96% | 90.31% |
| | 3 | 2.30E+12 | 2.30E+12 | 86.91% | 93.01% |
| | 7 | 2.29E+12 | 2.40E+12 | 86.50% | 97.17% |
| | 28 | 22.49E+12 | 2.46E+12 | 93.96% | 99.31% |
| RT | 1 | 2.22E+12 | 2.33E+12 | 83.65% | 94.12% |
| | 3 | 2.33E+12 | 2.20E+12 | 88.05% | 89.03% |
| | 7 | 2.34E+12 | 2.39E+12 | 88.07% | 96.67% |
| | 28 | 2.37E+12 | 2.19E+12 | 89.35% | 88.75% |

### Example 32-Protection From SARS-CoV-2 Challenge in Cynomolgus Macaques Vaccinated With AC 1

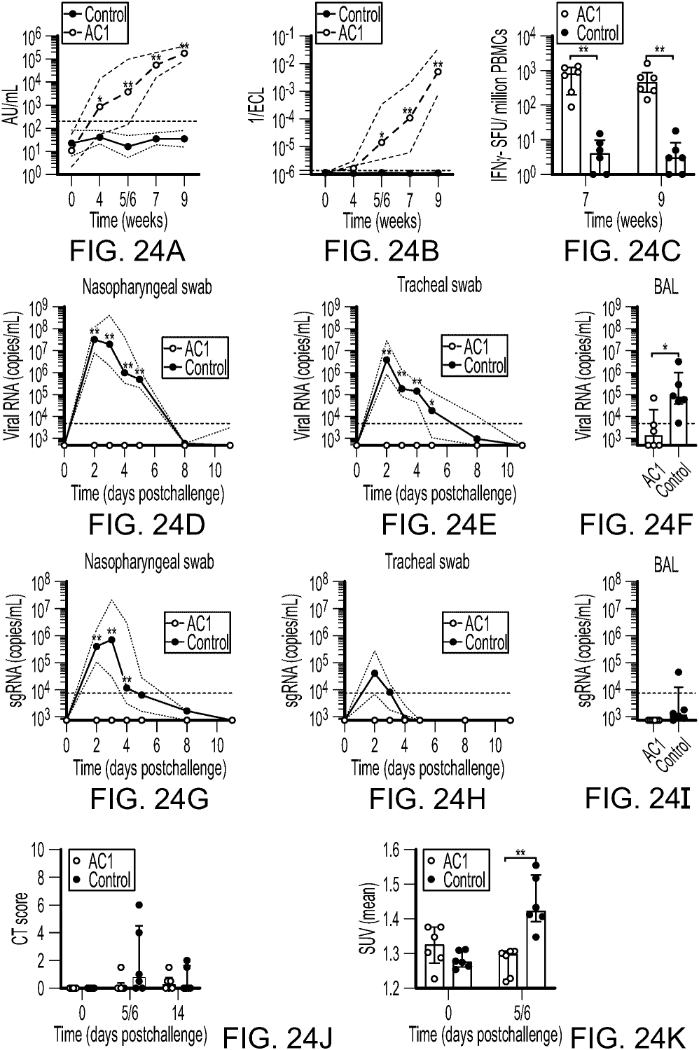

FIG. 24A is a graph showing the SARS-CoV-2 Spike ectodomain-binding IgG (measured in absorbance units (AU)/mL) elicited by AC1 in Cynomolgus macaques (n=6) during 9 weeks after vaccination. The dotted line indicates the lower detection limit of the assay.

FIG. 24B shows the measurement of antibodies that inhibit binding of Spike to ACE2 in an in vitro binding inhibition assay. The dotted line indicates the lower detection limit of the assay.

The data shown in FIGS. 24A-24B are represented as geometric mean titer (GMT) ± geometric SD. Mann Whitney test was used to compare vaccinated and control groups. The data shown in FIGS. 24C-24K are represented as median ± interquartile range. Mann Whitney test was used to compare vaccinated and control groups. * p<0.05, ** p<0.01.

FIG. 24C shows the quantification of IFN-gamma spot forming units (SFU) by ELISpot in PBMC samples collected at different timepoints in unvaccinated (Control) and AC1-treated cynomolgus macaques (n=6) and stimulated with peptides of SARS-CoV-2 Spike protein.

FIGS. 24D-24F shows the SARS-CoV-2 viral RNA copies in nasopharyngeal (24D) and tracheal swab (24E) at several time points after 10e5 pfu SARS-CoV-2 challenge and in bronchoalveolar lavage (BAL) fluid (24F) at day 3 after challenge.

FIGS. 24G-24I shows the SARS-CoV-2 subgenomic RNA quantification (copies/mL) in copies in nasopharyngeal (24G) and tracheal swab (24H) at several time points after 10e5 pfu SARS-CoV-2 challenge and in bronchoalveolar lavage (BAL) fluid (24I) at day 3 after challenge.

FIG. 24J shows the CT score in lungs of control and vaccinated animals before and after challenge. Scores were calculated based on lesion type (scored from 0 to 3) and lesion volume (scored from 0 to 4) for each lobe.

FIG. 24K shows measurement of lung lymph node (LN) activation measured by PET as mean standardized uptake values (SUV mean) before and after challenge.

### Example 33—Room Temperature Stability of AAVCOVID

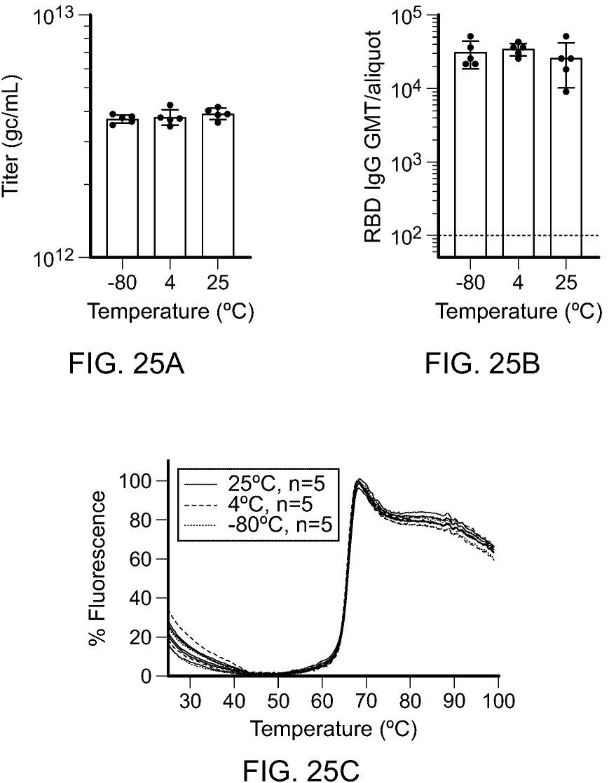

The stability of AC1 at different temperatures over a 1 month time period is shown in FIG. 25. FIG. 25A shows the titer (gc/mL) of AC1 aliquots (n=5) stored at -80° C., 4° C. and 25° C. for 4 weeks. FIG. 25B shows the RBD-binding antibody titers in C57BL/6 animals 21 days after vaccination with 1011gc of AC1 aliquots (n=5) stored at -80° C., 4° C. and 25° C. for 4 weeks. FIG. 25C shows AAV-ID analysis of capsid identity in AC1 aliquots (n=5) stored at -80° C., 4° C. and 25° C. for 4 weeks. These experiments demonstrate that AAVCOVID is stable at room temperature for at least one month.

### Example 34—Immunogenicity Via Different Administration Routes of AAVCOVID

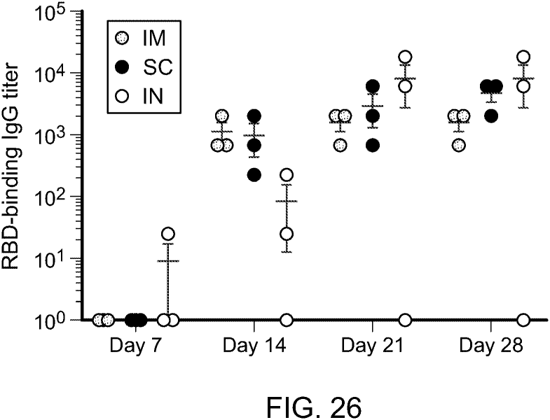

The immunogenicity of AC3 when administered via difference routes is shown in FIG. 26. FIG. 26 is a graph that shows the monitoring of SARS-CoV-2 RBD-binding IgG titers in 7-8 week-old C57BL/6 mice injected intramuscularly (IM), subcutaneously (SC) or intranasally (IN) with 5 × 10e10 gc AC3, n=3.

### Example 35-Characterization of AAV 11 for Use as a Vaccine

AAV was originally isolated from cynomolgus monkeys. AAV2/11 transduction in vitro is 1/100 compared to the AAV2 serotype. 1e9 genome copies of AAV per animal were systemically administered via the tail. After 1 week, the AAV vector was found in brain, lung, heart, liver, stomach, intestine, spleen, kidney, uterus, and muscle. After 6 weeks, AAV was found in muscle, kidney, spleen, lung, heart and stomach. Notably, only marginal expression in liver was observed.

AAV11 serotype was chosen for vaccine development as it is similar in sequence to Rh32.33, the AAV serotype used in the development of an AAVCOVID as described herein.

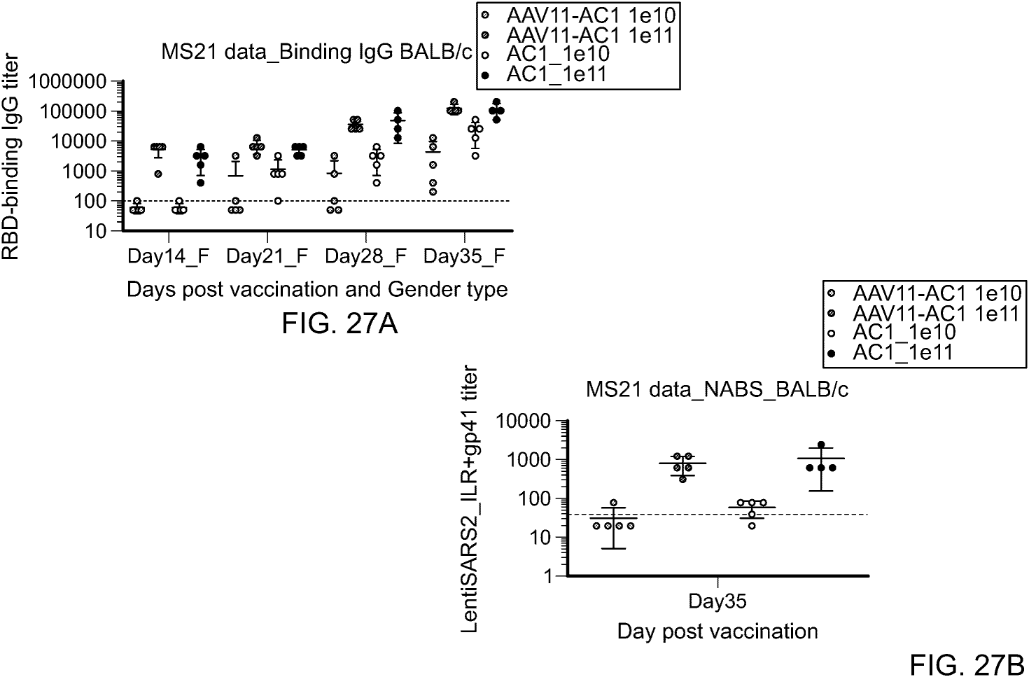

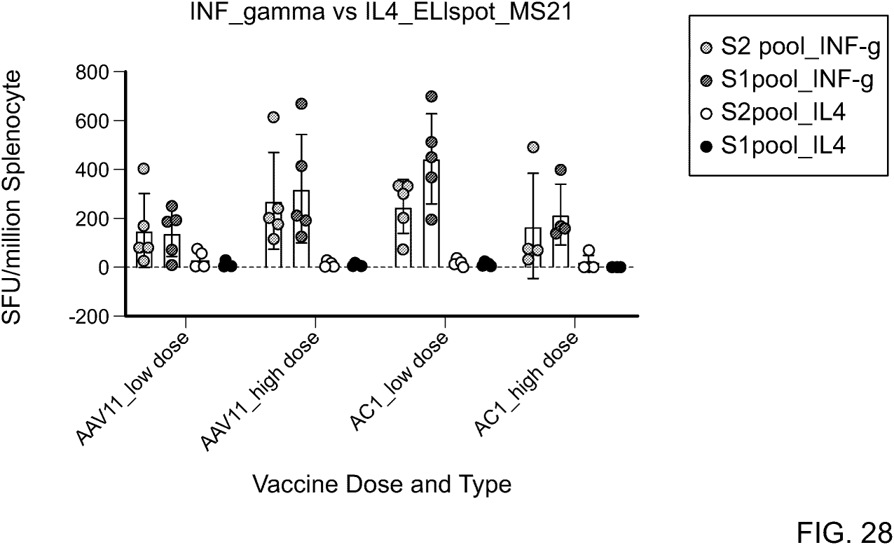

MS21 describes a short term study for measuring immunogenicity in BALB/c mice against SARS-CoV-2 full length stabilized Spike vaccinated with AAVCOVID AAV11 as compared to AAVCOVID Rh32.33. 5 female BALB/c mice were IM administered 1e10 or 1e11 AAV11-AC1 or AC1 (B857X), and blood was collected just before injection (baseline) and at days 14, 21 and 28. FIGS. 27A and 27B and FIG. 28 are graphs that show the results of these experiments.

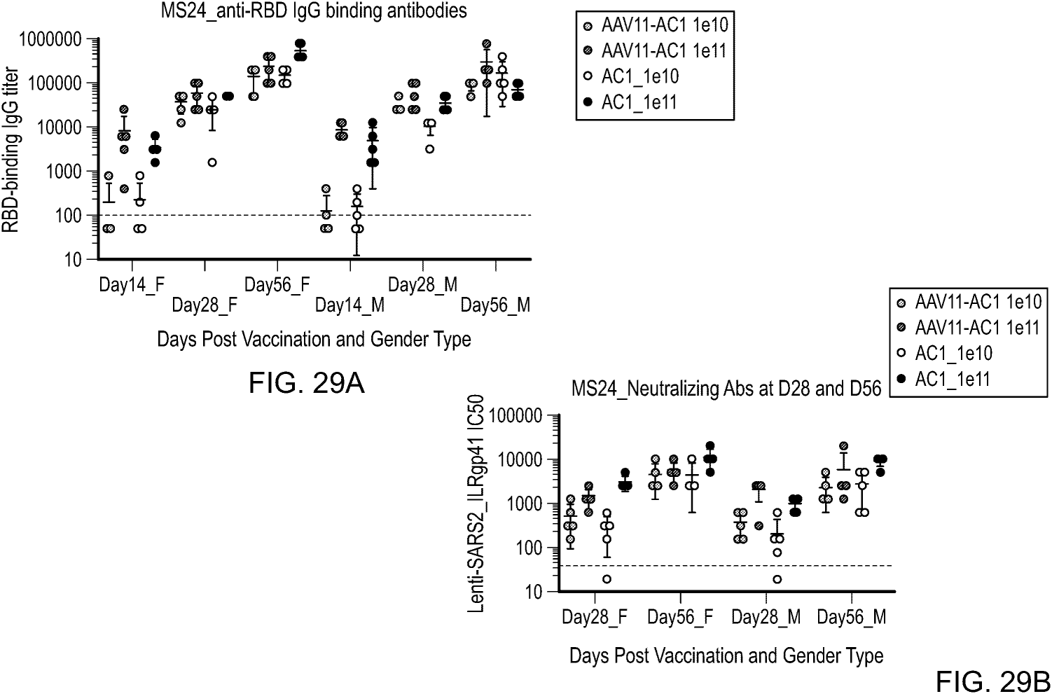

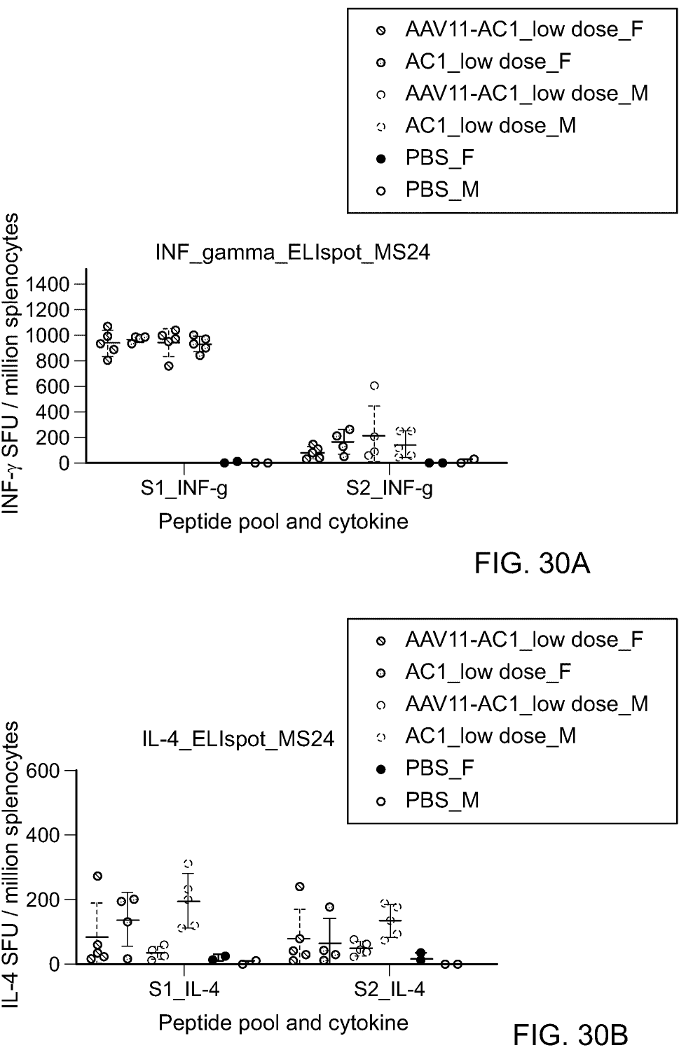

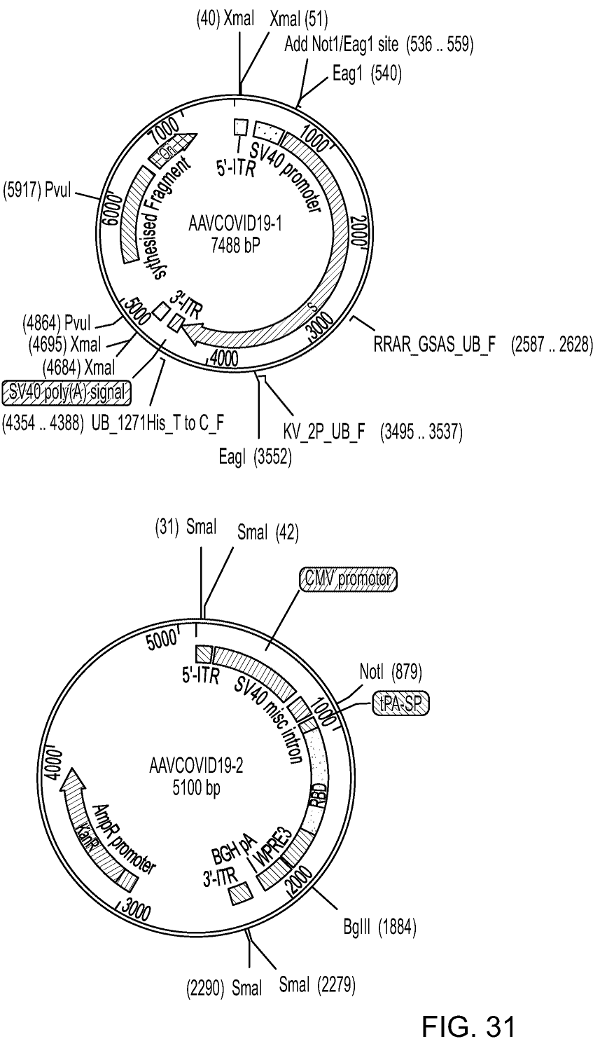

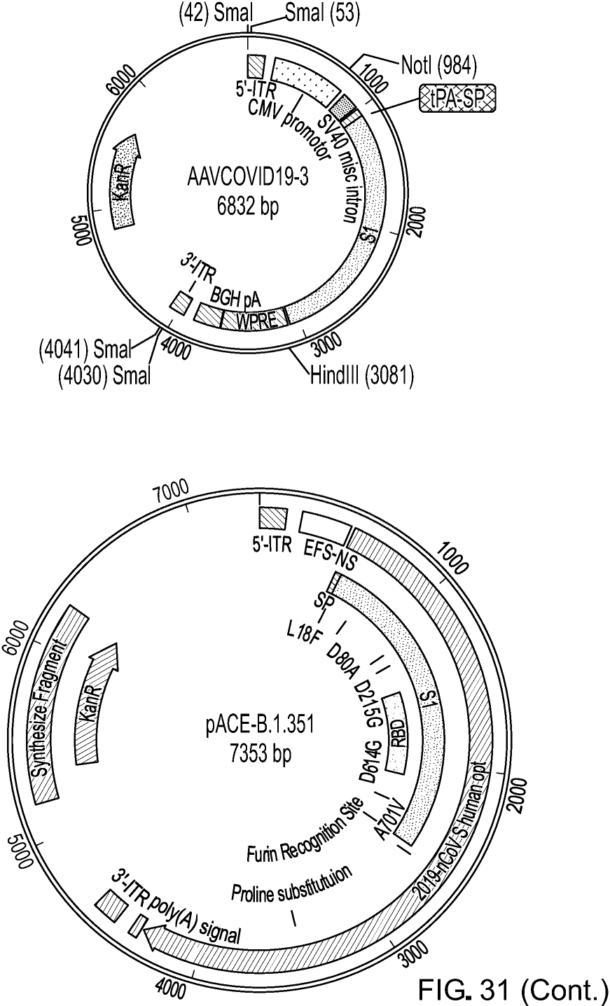

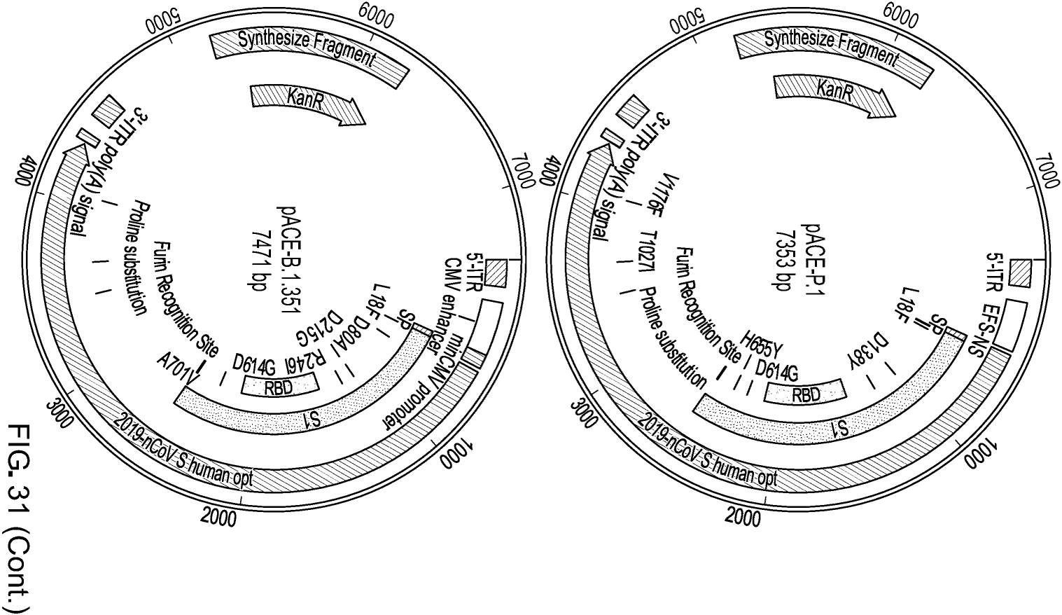

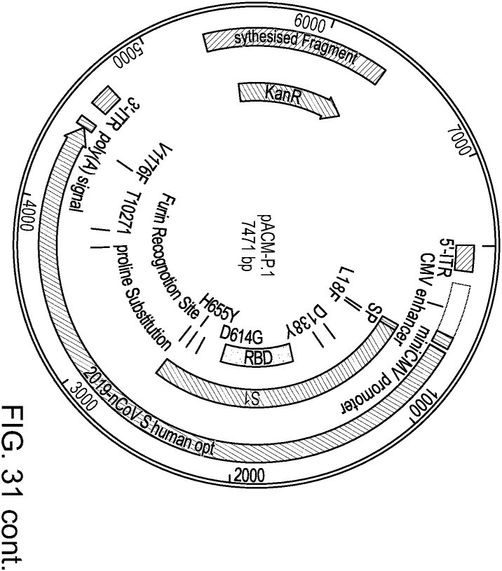

MS24 describes a long term study for measuring immunogenicity in C57BL/6 mice against SARS-CoV-2 full length stabilized Spike vaccinated with AAVCOVID AAV11 as compared to AAVCOVID Rh32.33. 5 female and 5 male BALB/c mice were IM administered 1e10 or 1e11 AAV1 1-AC1 or AC1, and blood was collected just before injection (baseline) and at days 14, 28, 42, 56, and at sacrifice at day 71. FIGS. 29A and 29B and FIGS. 30A and 30B are graphs that show the results of these experiments.

### Example 36—Sequence Information

With respect to SEQ ID NO:23:

| Feature | Begins and Ends | Total length |
|---|---|---|
| 5′-ITR | 1..141 | 141 |
| SV40 promoter | 222..538 | 317 |
| SV40 ori | 389..524 | 136 |
| FL stable spike | 559..4380 | 3822 |
| Signal Peptide | 559..597 | 39 |
| S1 | 598..2610 | 2013 |
| RBD | 1516..2181 | 666 |
| Furin Recognition Site | 2603..2610 | 8 |
| Proline substitution | 3519..3519 | 1 |
| SV40 poly A | 4390..4511 | 122 |
| 3′-ITR | 4599..4728 | 130 |
| synthesized Fragment | 5326..6379 | 1054 |
| KanR | 5492..6301 | 810 |
| Ori | 6472..7060 | 589 |

With respect to SEQ ID NO:25:

| Feature | Begins and Ends | Total length |
|---|---|---|
| 5′-ITR | 1..106 | 106 |
| CMV promotor | 117..699 | 583 |
| CMV enhancer | 177..480 | 304 |
| CMV promoter | 481..684 | 204 |
| SV40 intron | 737..869 | 133 |
| tPA-SP | 887..955 | 69 |
| Sc RBD | 956..1621 | 666 |
| WPRE3 | 1638..1883 | 246 |
| BGH pA | 1890..2104 | 215 |
| 3′-ITR | 2192..2321 | 130 |
| D-sequence | 2192..2211 | 20 |
| AmpR promoter | 2980..3084 | 105 |
| KanR | 3085..3894 | 810 |

With respect to SEQ ID NO:27:

| Feature | Begins and Ends | Total length |
|---|---|---|
| 5′-ITR | 1..141 | 141 |
| CMV promotor | 222..804 | 583 |
| SV40 intron | 842..974 | 133 |
| tPA-SP | 992..1060 | 69 |
| S1 | 1061..3076 | 2016 |
| WPRE | 3093..3634 | 542 |
| BGH pA | 3641..3855 | 215 |
| 3′-ITR | 3943..4072 | 130 |
| KanR | 4836..5645 | 810 |

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

SEQ ID NO: 1

ggcCTTAATTAGGCtgcgcgctcgctcgctcactgaggccgcccgggcaa
agcccgggcgtcgggcgacctttggtcgcccggcctcagtgagcgagcga
gcgcgcagagagggagtggccaactccatcactaggggttccttgtagtt
aatgattaacccgccatgctacttatctacgtagccatgctctaggaaga
tcggaattcgcccttaagctagctagttattaatagtaatcaattacggg
gtcattattcatagcccatatatggagttccgcgttacataacttacggt
aaatggcccgcctggctgaccgcccaacgacccccgcccattgacgtcaa
taatgacgtatgttcccatagtaacgccaatagggactttccattgacgt
caatgggtggagtatttacggtaaactgcccacttggcagtacatcaagt
gtatcatatgccaagtacgccccctattgacgtcaatgacggtaaatggc
ccgcctggcattatgcccagtacatgaccttatgggactttcctacttgg
cagtacatctacgtattagtcatcgctattaccatggtgatgcggttttg
gcagtacatcaatgggcgtggatagcggtttgactcacggggatttccaa
gtctccaccccattgacgtcaatgggagtttgttttggcaccaaaatcaa
cgggactttccaaaatgtcgtaacaactccgccccattgacgcaaatggg
cggtaggcgtgtacggtgggaggtctatataagcagagctggtttagtga
accgtcagatcctgcagaagttggtcgtgaggcactgggcaggtaagtat
caaggttacaagacaggtttaaggagaccaatagaaactgggcttgtcga
gacagagaagactcttgcgtttctgataggcacctattggtcttactgac
atccactttgcctttctctccacaggtgtccaggcggccgccatggatgc
aatgaagagagggctctgctgtgtgctgctgctgtgtggagcagtcttcg
tttcggccagcCAGTGCGTCAATCTGACAACTCGGACTCAGCTGCCACCT
GCTTATACTAATAGCTTCACCAGAGGCGTGTACTATCCTGACAAGGTGTT
TAGAAGCTCCGTGCTGCACTCTACACAGGATCTGTTTCTGCCATTCTTTA
GCAACGTGACCTGGTTCCACGCCATCCACGTGAGCGGCACCAATGGCACA
AAGCGGTTCGACAATCCCGTGCTGCCTTTTAACGATGGCGTGTACTTCGC
CTCTACCGAGAAGAGCAACATCATCAGAGGCTGGATCTTTGGCACCACAC
TGGACTCCAAGACACAGTCTCTGCTGATCGTGAACAATGCCACCAACGTG
GTCATCAAGGTGTGCGAGTTCCAGTTTTGTAATGATCCCTTCCTGGGCGT
GTACTATCACAAGAACAATAAGAGCTGGATGGAGTCCGAGTTTAGAGTGT
ATTCTAGCGCCAACAACTGCACATTTGAGTACGTGAGCCAGCCTTTCCTG
ATGGACCTGGAGGGCAAGCAGGGCAATTTCAAGAACCTGAGGGAGTTCGT
GTTTAAGAATATCGACGGCTACTTCAAAATCTACTCTAAGCACACCCCCA
TCAACCTGGTGCGCGACCTGCCTCAGGGCTTCAGCGCCCTGGAGCCCCTG
GTGGATCTGCCTATCGGCATCAACATCACCCGGTTTCAGACACTGCTGGC
CCTGCACAGAAGCTACCTGACACCCGGCGACTCCTCTAGCGGATGGACCG
CCGGCGCTGCCGCCTACTATGTGGGCTACCTCCAGCCCCGGACCTTCCTG
CTGAAGTACAACGAGAATGGCACCATCACAGACGCAGTGGATTGCGCCCT
GGACCCCCTGAGCGAGACAAAGTGTACACTGAAGTCCTTTACCGTGGAGA
AGGGCATCTATCAGACATCCAATTTCAGGGTGCAGCCAACCGAGTCTATC
GTGCGCTTTCCTAATATCACAAACCTGTGCCCATTTGGCGAGGTGTTCAA
CGCAACCCGCTTCGCCAGCGTGTACGCCTGGAATAGGAAGCGGATCAGCA
ACTGCGTGGCCGACTATAGCGTGCTGTACAACTCCGCCTCTTTCAGCACC
TTTAAGTGCTATGGCGTGTCCCCCACAAAGCTGAATGACCTGTGCTTTAC
CAACGTCTACGCCGATTCTTTCGTGATCAGGGGCGACGAGGTGCGCCAGA
TCGCCCCCGGCCAGACAGGCAAGATCGCAGACTACAATTATAAGCTGCCA
GACGATTTCACCGGCTGCGTGATCGCCTGGAACAGCAACAATCTGGATTC
CAAAGTGGGCGGCAACTACAATTATCTGTACCGGCTGTTTAGAAAGAGCA
ATCTGAAGCCCTTCGAGAGGGACATCTCTACAGAAATCTACCAGGCCGGC
AGCACCCCTTGCAATGGCGTGGAGGGCTTTAACTGTTATTTCCCACTCCA
GTCCTACGGCTTCCAGCCCACAAACGGCGTGGGCTATCAGCCTTACCGCG
TGGTGGTGCTGAGCTTTGAGCTGCTGCACGCCCCAGCAACAGTGTGCGGC
CCCAAGAAGTCCACCAATCTGGTGAAGAACAAGTGCGTGAACTTCAACTT
CAACGGCCTGACCGGCACAGGCGTGCTGACCGAGTCCAACAAGAAGTTCC
TGCCATTTCAGCAGTTCGGCAGGGACATCGCAGATACCACAGACGCCGTG
CGCGACCCACAGACCCTGGAGATCCTGGACATCACACCCTGCTCTTTCGG
CGGCGTGAGCGTGATCACACCCGGCACCAATACAAGCAACCAGGTGGCCG
TGCTGTATCAGGACGTGAATTGTACCGAGGTGCCCGTGGCTATCCACGCC
GATCAGCTGACCCCAACATGGCGGGTGTACAGCACCGGCTCCAACGTCTT
CCAGACAAGAGCCGGATGCCTGATCGGAGCAGAGCACGTGAACAATTCCT
ATGAGTGCGACATCCCAATCGGCGCCGGCATCTGTGCCTCTTACCAGACC
CAGACAAACTCTCCCAGAAGAGCCCGGtaataagcttggatccaatcaac
ctctggattacaaaattlgtgaaagattgactggtattcttaactatgti
gctccttttacgctatgtggatacgctgctttaatgcctttgtatcatgc
tattgcttcccgtatggctttcattttctcctccttgtataaatcctggt
tgctgtctctttatgaggagttgtggcccgttgtcaggcaacgtggcgtg
gtgtgcactgtgtttgctgacgcaacccccactggttggggcattgccac
cacctgtcagctcctttccgggactttcgctttccccctccctattgcca
cggcggaactcatcgccgcctgccttgcccgctgctggacaggggctcgg
ctgttgggcactgacaattccgtggtgttgtcggggaaatcatcgtcctt
tccttggctgctcgcctgtgttgccacctggattctgcgcgggacgtcct
tctgctacgtcccttcggccctcaatccagcggaccttccttcccgcggc
ctgctgccggctctgcggcctcttccgcgtcttcgagatctgcctcgact
gtgccttctagttgccagccatctgttgtttgcccctcccccgtgccttc
cttgaccctggaaggtgccactcccactgtcctttcctaataaaatgagg
aaattgcatcgcattgtctgagtaggtgtcattctattctggggggtggg
gtggggcaggacagcaagggggaggattgggaagacaatagcaggcatgc
tggggactcgagttaagggcgaattcccgataaggatcttcctagagcat
ggctacgtagataagtagcatggcgggttaatcattaactacaaggaacc
cctagtgatggagttggccactccctctctgcgcgctcgctcgctcactg
aggccgggcgaccaaaggtcgcccgacgcccgggctttgcccgggcggcc
tcagtgagcgagcgagcgcgcagccttaa

SEQ ID NO:2

ctgcgcgctcgctcgctcactgaggccgcccgggcaaagcccgggcgtcg
ggcgacctttggtcgcccggcctcagtgagcgagcgagcgcgcagagagg
gagtggttaagctagctagttattaatagtaatcaattacggggtcatta
gttcatagcccatatatggagttccgcgttacataacttacggtaaatgg
cccgcctggctgaccgcccaacgacccccgcccattgacgtcaataatga
cgtatgttcccatagtaacgccaatagggactttccattgacgtcaatgg
gtggagtatttacggtaaactgcccacttggcagtacatcaagtgtatca
tatgccaagtacgccccctattgacgtcaatgacggtaaatggcccgcct
ggcattatgcccagtacatgaccttatgggactttcctacttggcagtac
atctacgtattagtcatcgctattaccatggtgatgcggttttggcagta
catcaatgggcgtggatagcggtttgactcacggggatttccaagtctcc
accccattgacgtcaatgggagtttgttttggcaccaaaatcaacgggac
mccaaaatgtcgtaacaactccgccccattgacgcaaatgggcggtaggc
gtgtacggtgggaggtctatataagcagagctggtttagtgaaccgtcag
atcctgcagaagttggtcgtgaggcactgggcaggtaagtatcaaggtta
caagacaggtttaaggagaccaatagaaactgggcttgtcgagacagaga
agactcttgcgtttctgataggcacctattggtcttactgacatccactt
tgcctttctctccacaggtgtccaggcggccgccatggatgcaatgaaga
gagggctctgctgtgtgctgctgctgtgtggagcagtcttcgtttcggcc
agcGTGCAGCCAACCGAGTCTATCGTGCGCTTTCCTAATATCACAAACCT
GTGCCCATTTGGCGAGGTGTTCAACGCAACCCGCTTCGCCAGCGTGTACG
CCTGGAATAGGAAGCGGATCAGCAACTGCGTGGCCGACTATAGCGTGCTG
TACAACTCCGCCTCTTTCAGCACCTTTAAGTGCTATGGCGTGTCCCCCAC
AAAGCTGAATGACCTGTGCTTTACCAACGTCTACGCCGATTCTTTCGTGA
TCAGGGGCGACGAGGTGCGCCAGATCGCCCCCGGCCAGACAGGCAAGATC
GCAGACTACAATTATAAGCTGCCAGACGATTTCACCGGCTGCGTGATCGC
CTGGAACAGCAACAATCTGGATTCCAAAGTGGGCGGCAACTACAATTATC
TGTACCGGCTGTTTAGAAAGAGCAATCTGAAGCCCTTCGAGAGGGACATC
TCTACAGAAATCTACCAGGCCGGCAGCACCCCTTGCAATGGCGTGGAGGG
CTTTAACTGTTATTTCCCACTCCAGTCCTACGGCTTCCAGCCCACAAACG
GCGTGGGCTATCAGCCTTACCGCGTGGTGGTGCTGAGCTTTGAGCTGCTG
CACGCCCCAGCAACAGTGTGCGGCCCCAAGAAGTCCACCAATCTGGTGAA
GAACAAGTGCGTGAACTTCtaataagcttggatccaatcaacctctggat
tacaaaatttgtgaaagattgactggtattcttaactatgttgctccttt
tacgctatgtggatacgctgctttaatgcctttgtatcatgctattgctt
cccgtatggctttcattttctcctccttgtataaatcctggttagttctt
gccacggcggaactcatcgccgcctgccttgcccgctgctggacaggggc
tcggctgttgggcactgacaattccgtggtgagatctgcctcgactgtgc
cttctagttgccagccatctgttgtttgcccctcccccgtgccttccttg
accctggaaggtgccactcccactgtcctttcctaataaaatgaggaaat
tgcatcgcattgtctgagtaggtgtcattctattctggggggtggggtgg
ggcaggacagcaagggggaggattgggaagacaatagcaggcatgctggg
gactcgagttaagggcgaattcccgataaggatcttcctagagcatggct
acgtagataagtagcatggcgggttaatcattaactacaaggaaccccta
gtgatggagttggccactccctctctgcgcgctcgctcgctcactgaggc
cgggcgaccaaaggtcgcccgacgcccgggctttgcccgggcggcctcag
tgagcgagcgagcgcgcagccttaattaacctaattcactggccgtcgtt
ttacaacgtcgtgactgggaaaaccctggcgttacccaacttaatcgc

SEQ ID NO:3

cCTTAATTAGGCtgcgcgctcgctcgctcactgaggccgcccgggcaaag
cccgggcgtcgggcgacctttggtcgcccggcctcagtgagcgagcgagc
gcgcagagagggagtggccaactccatcactaggggttccttgtagttaa
tgattaacccgccatgctacttatctacgtagccatgctctaggaagatc
ggaattcgcccttaagctagctagttattaatagtaatcaattacggggt
cattagttcatagcccatatatggagttccgcgttacataacttacggta
aatggcccgcctggctgaccgcccaacgacccccgcccattgacgtcaat
aatgacgtatgttcccatagtaacgccaatagggactttccattgacgtc
aatgggtggagtatttacggtaaactgcccacttggcagtacatcaagtg
tatcatatgccaagtacgccccctattgacgtcaatgacggtaaatggcc
cgcctggcattatgcccagtacatgaccttatgggactttcctacttggc
agtacatctacgtattagtcatcgctattaccatggtgatgcggttttgg
cagtacatcaatgggcgtggatagcggtttgactcacggggatttccaag
tctccaccccattgacgtcaatgggagtttgttttggcaccaaaatcaac
gggactttccaaaatgtcgtaacaactccgccccattgacgcaaatgggc
ggtaggcgtgtacggtgggaggtctatataagcagagctggtttagtgaa
ccgtcagatcctgcagaagttggtcgtgaggcactgggcaggtaagtatc
aaggttacaagacaggtttaaggagaccaatagaaactgggcttgtcgag
acagagaagactcttgcgtttctgataggcacctattggtcttactgaca
tccactttgcctttctctccacaggtgtccaggcggccgccatggatgca
atgaagagagggctctgctgtgtgctgctgctgtgtggagcagtcttcgt
ttcggccagcGTGCAGCCAACCGAGTCTATCGTGCGCTTTCCTAATATCA
CAAACCTGTGCCCATTTGGCGAGGTGTTCAACGCAACCCGCTTCGCCAGC
GTGTACGCCTGGAATAGGAAGCGGATCAGCAACTGCGTGGCCGACTATAG
CGTGCTGTACAACTCCGCCTCTTTCAGCACCTTTAAGTGCTATGGCGTGT
CCCCCACAAAGCTGAATGACCTGTGCTTTACCAACGTCTACGCCGATTCT
TTCGTGATCAGGGGCGACGAGGTGCGCCAGATCGCCCCCGGCCAGACAGG
CAAGATCGCAGACTACAATTATAAGCTGCCAGACGATTTCACCGGCTGCG
TGATCGCCTGGAACAGCAACAATCTGGATTCCAAAGTGGGCGGCAACTAC
AATTATCTGTACCGGCTGTTTAGAAAGAGCAATCTGAAGCCCTTCGAGAG
GGACATCTCTACAGAAATCTACCAGGCCGGCAGCACCCCTTGCAATGGCG
TGGAGGGCTTTAACTGTTATTTCCCACTCCAGTCCTACGGCTTCCAGCCC
ACAAACGGCGTGGGCTATCAGCCTTACCGCGTGGTGGTGCTGAGCTTTGA
GCTGCTGCACGCCCCAGCAACAGTGTGCGGCCCCAAGAAGTCCACCAATC
TGGTGAAGAACAAGTGCGTGAACTTCtaataagcttggatccaatcaacc
tctggattacaaaatttgtgaaagattgactggtattcttaactatgttg
ctccttttacgctatgtggatacgctgctttaatgcctttgtatcatgct
attgcttcccgtatggctttcattttctcctccttgtataaatcctggtt
agttcttgccacggcggaactcatcgccgcctgccttgcccgctgctgga
caggggctcggctgttgggcactgacaattccgtggtgagatctgcctcg
actgtgccttctagttgccagccatctgttgtttgcccctcccccgtgcc
ttccttgaccctggaaggtgccactcccactgtcctttcctaataaaatg
aggaaattgcatcgcattgtctgagtaggtgtcattctattctggggggt
ggggtggggcaggacagcaagggggaggattgggaagacaatagcaggca
tgctggggactcgagttaagggcgaattcccgataaggatcttcctagag
catggctacgtagataagtagcatggcgggttaatcattaactacaagga
acccctagtgatggagttggccactccctctctgcgcgctcgctcgctca
ctgaggccgggcgaccaaaggtcgcccgacgcccgggctttgcccgggcg
gcctcagtgagcgagcgagcgcgcagccttaattaacctaattcactggc
cgtcgttttacaacgtcgtgactgggaaaaccctggcgttacccaactta

SEQ ID NO:4

ctgcgcgctcgctcgctcactgaggccgcccgggcaaagcccgggcgtcg
ggcgacctttggtcgcccggcctcagtgagcgagcgagcgcgcagagagg
gagtggccaactccatcactaggggttccttgtagttaatgattaacccg
ccatgctacttatctacgtagccatgctctaggaagatcggaattcgccc
ttaagctagcGGTGTGGAAAGTCCCCAGGCTCCCCAGCAGGCAGAAGTAT
GCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCA
ACCATAGTCCCGCCCCTAACTCCGCCCATCCCGCCCCTAACTCCGCCCAG
TTCCGCCCATTCTCCGCCCCATGGCTGACTAATTTTTTTTATTTATGCAG
AGGCCGAGGCCGCCTCGGCCTCTGAGCTATTCCAGAAGTAGTGAGGAGGC
TTTTTTGGAGGCCTAGGCTTTTGCAAAgcggccgcctaagctCTAGCGTT
TAAACGGGCCCTCTAGATGCCACCATGTTCGTCTTCCTGGTCCTGCTGCC
TCTGGTCTCCTCACAGTGCGTCAATCTGACAACTCGGACTCAGCTGCCAC
CTGCTTATACTAATAGCTTCACCAGAGGCGTGTACTATCCTGACAAGGTG
TTTAGAAGCTCCGTGCTGCACTCTACACAGGATCTGTTTCTGCCATTCTT
TAGCAACGTGACCTGGTTCCACGCCATCCACGTGAGCGGCACCAATGGCA
CAAAGCGGTTCGACAATCCCGTGCTGCCTTTTAACGATGGCGTGTACTTC
GCCTCTACCGAGAAGAGCAACATCATCAGAGGCTGGATCTTTGGCACCAC
ACTGGACTCCAAGACACAGTCTCTGCTGATCGTGAACAATGCCACCAACG
TGGTCATCAAGGTGTGCGAGTTCCAGTTTTGTAATGATCCCTTCCTGGGC
GTGTACTATCACAAGAACAATAAGAGCTGGATGGAGTCCGAGTTTAGAGT
GTATTCTAGCGCCAACAACTGCACATTTGAGTACGTGAGCCAGCCTTTCC
TGATGGACCTGGAGGGCAAGCAGGGCAATTTCAAGAACCTGAGGGAGTTC
GTGTTTAAGAATATCGACGGCTACTTCAAAATCTACTCTAAGCACACCCC
CATCAACCTGGTGCGCGACCTGCCTCAGGGCTTCAGCGCCCTGGAGCCCC
TGGTGGATCTGCCTATCGGCATCAACATCACCCGGTTTCAGACACTGCTG
GCCCTGCACAGAAGCTACCTGACACCCGGCGACTCCTCTAGCGGATGGAC
CGCCGGCGCTGCCGCCTACTATGTGGGCTACCTCCAGCCCCGGACCTTCC
TGCTGAAGTACAACGAGAATGGCACCATCACAGACGCAGTGGATTGCGCC
CTGGACCCCCTGAGCGAGACAAAGTGTACACTGAAGTCCTTTACCGTGGA
GAAGGGCATCTATCAGACATCCAATTTCAGGGTGCAGCCAACCGAGTCTA
TCGTGCGCTTTCCTAATATCACAAACCTGTGCCCATTTGGCGAGGTGTTC
AACGCAACCCGCTTCGCCAGCGTGTACGCCTGGAATAGGAAGCGGATCAG
CAACTGCGTGGCCGACTATAGCGTGCTGTACAACTCCGCCTCTTTCAGCA
CCTTTAAGTGCTATGGCGTGTCCCCCACAAAGCTGAATGACCTGTGCTTT
ACCAACGTCTACGCCGATTCTTTCGTGATCAGGGGCGACGAGGTGCGCCA
GATCGCCCCCGGCCAGACAGGCAAGATCGCAGACTACAATTATAAGCTGC
CAGACGATTTCACCGGCTGCGTGATCGCCTGGAACAGCAACAATCTGGAT
TCCAAAGTGGGCGGCAACTACAATTATCTGTACCGGCTGTTTAGAAAGAG
CAATCTGAAGCCCTTCGAGAGGGACATCTCTACAGAAATCTACCAGGCCG
GCAGCACCCCTTGCAATGGCGTGGAGGGCTTTAACTGTTATTTCCCACTC
CAGTCCTACGGCTTCCAGCCCACAAACGGCGTGGGCTATCAGCCTTACCG
CGTGGTGGTGCTGAGCTTTGAGCTGCTGCACGCCCCAGCAACAGTGTGCG
GCCCCAAGAAGTCCACCAATCTGGTGAAGAACAAGTGCGTGAACTTCAAC
TTCAACGGCCTGACCGGCACAGGCGTGCTGACCGAGTCCAACAAGAAGTT
CCTGCCATTTCAGCAGTTCGGCAGGGACATCGCAGATACCACAGACGCCG
TGCGCGACCCACAGACCCTGGAGATCCTGGACATCACACCCTGCTCTTTC
GGCGGCGTGAGCGTGATCACACCCGGCACCAATACAAGCAACCAGGTGGC
CGTGCTGTATCAGGACGTGAATTGTACCGAGGTGCCCGTGGCTATCCACG
CCGATCAGCTGACCCCAACATGGCGGGTGTACAGCACCGGCTCCAACGTC
TTCCAGACAAGAGCCGGATGCCTGATCGGAGCAGAGCACGTGAACAATTC
CTATGAGTGCGACATCCCAATCGGCGCCGGCATCTGTGCCTCTTACCAGA
CCCAGACAAACTCTCCCAGAAGAGCCCGGAGCGTGGCCTCCCAGTCTATC
ATCGCCTATACCATGTCCCTGGGCGCCGAGAACAGCGTGGCCTACTCTAA
CAATAGCATCGCCATCCCAACCAACTTCACAATCTCTGTGACCACAGAGA
TCCTGCCCGTGTCCATGACCAAGACATCTGTGGACTGCACAATGTATATC
TGTGGCGATTCTACCGAGTGCAGCAACCTGCTGCTCCAGTACGGCAGCTT
TTGTACCCAGCTGAATAGAGCCCTGACAGGCATCGCCGTGGAGCAGGATA
AGAACACACAGGAGGTGTTCGCCCAGGTGAAGCAAATCTACAAGACCCCC
CCTATCAAGGACTTTGGCGGCTTCAATTTTTCCCAGATCCTGCCTGATCC
ATCCAAGCCTTCTAAGCGGAGCTTTATCGAGGACCTGCTGTTCAACAAGG
TGACCCTGGCCGATGCCGGCTTCATCAAGCAGTATGGCGATTGCCTGGGC
GACATCGCAGCCAGGGACCTGATCTGCGCCCAGAAGTTTAATGGCCTGAC
CGTGCTGCCACCCCTGCTGACAGATGAGATGATCGCACAGTACACAAGCG
CCCTGCTGGCCGGCACCATCACATCCGGATGGACCTTCGGCGCAGGAGCC
GCCCTCCAGATCCCCTTTGCCATGCAGATGGCCTATAGGTTCAACGGCAT
CGGCGTGACCCAGAATGTGCTGTACGAGAACCAGAAGCTGATCGCCAATC
AGTTTAACTCCGCCATCGGCAAGATCCAGGACAGCCTGTCCTCTACAGCC
AGCGCCCTGGGCAAGCTCCAGGATGTGGTGAATCAGAACGCCCAGGCCCT
GAATACCCTGGTGAAGCAGCTGAGCAGCAACTTCGGCGCCATCTCTAGCG
TGCTGAATGACATCCTGAGCCGGCTGGACAAGGTGGAGGCAGAGGTGCAG
ATCGACCGGCTGATCACCGGCCGGCTCCAGAGCCTCCAGACCTATGTGAC
ACAGCAGCTGATCAGGGCCGCCGAGATCAGGGCCAGCGCCAATCTGGCAG
CAACCAAGATGTCCGAGTGCGTGCTGGGCCAGTCTAAGAGAGTGGACTTT
TGTGGCAAGGGCTATCACCTGATGTCCTTCCCTCAGTCTGCCCCACACGG
CGTGGTGTTTCTGCACGTGACCTACGTGCCCGCCCAGGAGAAGAACTTCA
CCACAGCCCCTGCCATCTGCCACGATGGCAAGGCCCACTTTCCAAGGGAG
GGCGTGTTCGTGTCCAACGGCACCCACTGGTTTGTGACACAGCGCAATTT
CTACGAGCCCCAGATCATCACCACAGACAACACCTTCGTGAGCGGCAACT
GTGACGTGGTCATCGGCATCGTGAACAATACCGTGTATGATCCACTCCAG
CCCGAGCTGGACAGCTTTAAGGAGGAGCTGGATAAGTATTTCAAGAATCA
CACCTCCCCTGACGTGGATCTGGGCGACATCAGCGGCATCAATGCCTCCG
TGGTGAACATCCAGAAGGAGATCGACCGCCTGAACGAGGTGGCTAAGAAT
CTGAACGAGAGCCTGATCGACCTCCAGGAGCTGGGCAAGTATGAGCAGTA
CATCAAGTGGCCCTGGTACATCTGGCTGGGCTTCATCGCCGGCCTGATCG
CCATCGTGATGGTGACCATCATGCTGTGCTGTATGACATCCTGCTGTTCT
TGCCTGAAGGGCTGCTGTAGCTGTGGCTCCTGCTGTAAGTTTGACGAGGA
TGACTCTGAACCTGTGCTGAAGGGCGTGAAGCTGCATTACACCTAAATCG
GATCttaagatacattgatgagtttggacaaaccacaactagaatgcagt
gaaaaaaatgcmatttgtgaaatttgtgatgctattgctttatttgtaac
cattataagctgcaataaacaagttctcgagttaagggcgaattcccgat
aaggatcttcctagagcatggctacgtagataagtagcatggcgggttaa
tcattaactacaaggaacccctagtgatggagttggccactccctctctg
cgcgctcgctcgctcactgaggccgggcgaccaaaggtcgcccgacgccc
gggctttgcccgggcggcctcagtgagcgagcgagcgcgcagccttaatt
aacctaattcactggccgtcgttttacaacgtcgtgactgggaaaaccct
ggcgttac

SEQ ID NO:5

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVWLSFELLHAPATVCGPKKSTNLVKNKCVNFNFNGLTGTGV
LTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLI
GAEHVNNSYECDIPIGAGICASYQTQTNSPRRAR↓SVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

SEQ ID NO:6

IKRMKQIEDKIEEIESKQKKIENEIARIKKIK

SEQ ID NO:7

GSGYIPEAPRDGQAYVRKDGEWVLLSTFL

SEQ ID NO:8

GGTGGNGTGRMKQIEDKIENITSKIY NITNEIARIKKLIGNRT

SEQ ID NO:9

cCTTAATTAGGCtgcgcgctcgctcgctcactgaggccgcccgggcaaag
cccgggcgtcgggcgacctttggtcgcccggcctcagtgagcgagcgagc
gcgcagagagggagtggccaactccatcactaggggttccttgtagttaa
tgattaacccgccatgctacttatctacgtagccatgctctaggaagatc
ggaattcgcccttaagctagcGGTGTGGAAAGTCCCCAGGCTCCCCAGCA
GGCAGAAGTATGCAAAGCATGCATCTCAATTAGTCAGCAACCAGGTGTGG
AAAGTCCCCAGGCTCCCCAGCAGGCAGAAGTATGCAAAGCATGCATCTCA
ATTAGTCAGCAACCATAGTCCCGCCCCTAACTCCGCCCATCCCGCCCCTA
ACTCCGCCCAGTTCCGCCCATTCTCCGCCCCATGGCTGACTAATTTTTTT
TATTTATGCAGAGGCCGAGGCCGCCTCGGCCTCTGAGCTATTCCAGAAGT
AGTGAGGAGGCTTTTTTGGAGGCCTAGGCTTTTGCAAAgcggccgcctag
atgccaccaTGTTCGTCTTCCTGGTCCTGCTGCCTCTGGTCTCCTCACAG
TGCGTCAATCTGACAACTCGGACTCAGCTGCCACCTGCTTATACTAATAG
CTTCACCAGAGGCGTGTACTATCCTGACAAGGTGTTTAGAAGCTCCGTGC
TGCACTCTACACAGGATCTGTTTCTGCCATTCTTTAGCAACGTGACCTGG
TTCCACGCCATCCACGTGAGCGGCACCAATGGCACAAAGCGGTTCGACAA
TCCCGTGCTGCCTTTTAACGATGGCGTGTACTTCGCCTCTACCGAGAAGA
GCAACATCATCAGAGGCTGGATCTTTGGCACCACACTGGACTCCAAGACA
CAGTCTCTGCTGATCGTGAACAATGCCACCAACGTGGTCATCAAGGTGTG
CGAGTTCCAGTTTTGTAATGATCCCTTCCTGGGCGTGTACTATCACAAGA
ACAATAAGAGCTGGATGGAGTCCGAGTTTAGAGTGTATTCTAGCGCCAAC
AACTGCACATTTGAGTACGTGAGCCAGCCTTTCCTGATGGACCTGGAGGG
CAAGCAGGGCAATTTCAAGAACCTGAGGGAGTTCGTGTTTAAGAATATCG
ACGGCTACTTCAAAATCTACTCTAAGCACACCCCCATCAACCTGGTGCGC
GACCTGCCTCAGGGCTTCAGCGCCCTGGAGCCCCTGGTGGATCTGCCTAT
CGGCATCAACATCACCCGGTTTCAGACACTGCTGGCCCTGCACAGAAGCT
ACCTGACACCCGGCGACTCCTCTAGCGGATGGACCGCCGGCGCTGCCGCC
TACTATGTGGGCTACCTCCAGCCCCGGACCTTCCTGCTGAAGTACAACGA
GAATGGCACCATCACAGACGCAGTGGATTGCGCCCTGGACCCCCTGAGCG
AGACAAAGTGTACACTGAAGTCCTTTACCGTGGAGAAGGGCATCTATCAG
ACATCCAATTTCAGGGTGCAGCCAACCGAGTCTATCGTGCGCTTTCCTAA
TATCACAAACCTGTGCCCATTTGGCGAGGTGTTCAACGCAACCCGCTTCG
CCAGCGTGTACGCCTGGAATAGGAAGCGGATCAGCAACTGCGTGGCCGAC
TATAGCGTGCTGTACAACTCCGCCTCTTTCAGCACCTTTAAGTGCTATGG
CGTGTCCCCCACAAAGCTGAATGACCTGTGCTTTACCAACGTCTACGCCG
ATTCTTTCGTGATCAGGGGCGACGAGGTGCGCCAGATCGCCCCCGGCCAG
ACAGGCAAGATCGCAGACTACAATTATAAGCTGCCAGACGATTTCACCGG
CTGCGTGATCGCCTGGAACAGCAACAATCTGGATTCCAAAGTGGGCGGCA
ACTACAATTATCTGTACCGGCTGTTTAGAAAGAGCAATCTGAAGCCCTTC
GAGAGGGACATCTCTACAGAAATCTACCAGGCCGGCAGCACCCCTTGCAA
TGGCGTGGAGGGCTTTAACTGTTATTTCCCACTCCAGTCCTACGGCTTCC
AGCCCACAAACGGCGTGGGCTATCAGCCTTACCGCGTGGTGGTGCTGAGC
TTTGAGCTGCTGCACGCCCCAGCAACAGTGTGCGGCCCCAAGAAGTCCAC
CAATCTGGTGAAGAACAAGTGCGTGAACTTCAACTTCAACGGCCTGACCG
GCACAGGCGTGCTGACCGAGTCCAACAAGAAGTTCCTGCCATTTCAGCAG
TTCGGCAGGGACATCGCAGATACCACAGACGCCGTGCGCGACCCACAGAC
CCTGGAGATCCTGGACATCACACCCTGCTCTTTCGGCGGCGTGAGCGTGA
TCACACCCGGCACCAATACAAGCAACCAGGTGGCCGTGCTGTATCAGGAC
GTGAATTGTACCGAGGTGCCCGTGGCTATCCACGCCGATCAGCTGACCCC
AACATGGCGGGTGTACAGCACCGGCTCCAACGTCTTCCAGACAAGAGCCG
GATGCCTGATCGGAGCAGAGCACGTGAACAATTCCTATGAGTGCGACATC
CCAATCGGCGCCGGCATCTGTGCCTCTTACCAGACCcagacaaactctcc
cggaagcgccagtagcgtggcctcccagTCTATCATCGCCTATACCATGT
CCCTGGGCGCCGAGAACAGCGTGGCCTACTCTAACAATAGCATCGCCATC
CCAACCAACTTCACAATCTCTGTGACCACAGAGATCCTGCCCGTGTCCAT
GACCAAGACATCTGTGGACTGCACAATGTATATCTGTGGCGATTCTACCG
AGTGCAGCAACCTGCTGCTCCAGTACGGCAGCTTTTGTACCCAGCTGAAT
AGAGCCCTGACAGGCATCGCCGTGGAGCAGGATAAGAACACACAGGAGGT
GTTCGCCCAGGTGAAGCAAATCTACAAGACCCCCCCTATCAAGGACTTTG
GCGGCTTCAATTTTTCCCAGATCCTGCCTGATCCATCCAAGCCTTCTAAG
CGGAGCTTTATCGAGGACCTGCTGTTCAACAAGGTGACCCTGGCCGATGC
CGGCTTCATCAAGCAGTATGGCGATTGCCTGGGCGACATCGCAGCCAGGG
ACCTGATCTGCGCCCAGAAGTTTAATGGCCTGACCGTGCTGCCACCCCTG
CTGACAGATGAGATGATCGCACAGTACACAAGCGCCCTGCTGGCCGGCAC
CATCACATCCGGATGGACCTTCGGCGCAGGAGCCGCCCTCCAGATCCCCT
TTGCCATGCAGATGGCCTATAGGTTCAACGGCATCGGCGTGACCCAGAAT
GTGCTGTACGAGAACCAGAAGCTGATCGCCAATCAGTTTAACTCCGCCAT
CGGCAAGATCCAGGACAGCCTGTCCTCTACAGCCAGCGCCCTGGGCAAGC
TCCAGGATGTGGTGAATCAGAACGCCCAGGCCCTGAATACCCTGGTGAAG
CAGCTGAGCAGCAACTTCGGCGCCATCTCTAGCGTGCTGAATGAcatcct
gagccggctggacccaccggaggcagaggtgcagatcGACCGGCTGATCA
CCGGCCGGCTCCAGAGCCTCCAGACCTATGTGACACAGCAGCTGATCAGG
GCCGCCGAGATCAGGGCCAGCGCCAATCTGGCAGCAACCAAGATGTCCGA
GTGCGTGCTGGGCCAGTCTAAGAGAGTGGACTTTTGTGGCAAGGGCTATC
ACCTGATGTCCTTCCCTCAGTCTGCCCCACACGGCGTGGTGTTTCTGCAC
GTGACCTACGTGCCCGCCCAGGAGAAGAACTTCACCACAGCCCCTGCCAT
CTGCCACGATGGCAAGGCCCACTTTCCAAGGGAGGGCGTGTTCGTGTCCA
ACGGCACCCACTGGTTTGTGACACAGCGCAATTTCTACGAGCCCCAGATC
ATCACCACAGACAACACCTTCGTGAGCGGCAACTGTGACGTGGTCATCGG
CATCGTGAACAATACCGTGTATGATCCACTCCAGCCCGAGCTGGACAGCT
TTAAGGAGGAGCTGGATAAGTATTTCAAGAATCACACCTCCCCTGACGTG
GATCTGGGCGACATCAGCGGCATCAATGCCTCCGTGGTGAACATCCAGAA
GGAGATCGACCGCCTGAACGAGGTGGCTAAGAATCTGAACGAGAGCCTGA
TCGACCTCCAGGAGCTGGGCAAGTATGAGCAGTACATCAAGTGGCCCTGG
TACATCTGGCTGGGCTTCATCGCCGGCCTGATCGCCATCGTGATGGTGAC
CATCATGCTGTGCTGTATGACATCCTGCTGTTCTTGCCTGAAGGGCTGCT
GTAGCTGTGGCTCCTGCTGTAAGTTTGACGAGGATGACTCTGAACCTGTG
CTGAAGGGCGTGAAGCTGCACTACACCTAAATCGgatcttaagatacatt
gatgagtttggacaaaccacaactagaatgcagtgaaaaaaatgctttat
ttgtgaaatttgtgatgctattgctttatttgtaaccattataagctgca
ataaacaagttctcgagttaagggcgaattcccgataaggatcttcctag
agcatggctacgtagataagtagcatggcgggttaatcattaactacaag
gaacccctagtgatggagttggccactccctctctgcgcgctcgctcgct
cactgaggccgggcgaccaaaggtcgcccgacgcccgggctttgcccggg
cggcctcagtgagcgagcgagcgcgcagccttaattaacctaattcactg
gccgtcgttttacaacgtcgtgactgggaaaaccctggcgttacccaact
taatcgccttgcagcacatccccctttcgccagctggcgtaatagcgaag
aggcccgcaccgatcgcccttcccaacagttgcgcagcctgaatggcgaa
tgggacgcgccctgtagcggcgcattaagcgcggcgggtgtggtggttac
gcgcagcgtgaccgctacacttgccagcgccctagcgcccgctcctttcg
ctttcttcccttcctttctcgccacgttcgccggctttccccgtcaagct
ctaaatcgggggctccctttagggttccgatttagtgctttacggcacct
cgaccccaaaaaacttgattagggtgatggttcacgtagtgggccatcgc
cctgatagacggtttttcgccctttgacgttggagtccacgttctttaat
agtggactcttgttccaaactggaacaacactcaaccctatctcggtcta
ttcttttgatttataagggattttgccgatttcggcctattggttaaaaa
atgagctgatttaacaaaaatttaacgcgaattttaacaaaatattaacg
tttataatttcaggtggcatctttcggggaaatgtgcgcggaacccctat
ttgtttatttttctaaatacattcaaatatgtatccgctcatgagacaat
aaccctgataaatgcttcaataatattgaaaaaggaagagtATGAGCCAT
ATTCAACGGGAAACGTCGAGGCCGCGATTAAATTCCAACATGGATGCTGA
TTTATATGGGTATAAATGGGCTCGCGATAATGTCGGGCAATCAGGTGCGA
CAATCTATCGCTTGTATGGGAAGCCCGATGCGCCAGAGTTGTTTCTGAAA
CATGGCAAAGGTAGCGTTGCCAATGATGTTACAGATGAGATGGTCAGACT
AAACTGGCTGACGGAATTTATGCCTCTTCCGACCATCAAGCATTTTATCC
GTACTCCTGATGATGCATGGTTACTCACCACTGCGATCCCCGGAAAAACA
GCATTCCAGGTATTAGAAGAATATCCTGATTCAGGTGAAAATATTGTTGA
TGCGCTGGCAGTGTTCCTGCGCCGGTTGCATTCGATTCCTGTTTGTAATT
GTCCTTTTAACAGCGATCGCGTATTTCGTCTCGCTCAGGCGCAATCACGA
ATGAATAACGGTTTGGTTGATGCGAGTGATTTTGATGACGAGCGTAATGG
CTGGCCTGTTGAACAAGTCTGGAAAGAAATGCATAAACTTTTGCCATTCT
CACCGGATTCAGTCGTCACTCATGGTGATTTCTCACTTGATAACCTTATT
TTTGACGAGGGGAAATTAATAGGTTGTATTGATGTTGGACGAGTCGGAAT
CGCAGACCGATACCAGGATCTTGCCATCCTATGGAACTGCCTCGGTGAGT
TTTCTCCTTCATTACAGAAACGGCTTTTTCAAAAATATGGTATTGATAAT
CCTGATATGAATAAATTGCAGTTTCATTTGATGCTCGATGAGTTTTTCTA
Actgtcagaccaagtttactcatatatactttagattgatttaaaacttc
atttttaatttaaaaggatctaggtgaagatcctttttgataatctcatg
accaaaatcccttaacgtgagttttcgttccactgagcgtcagaccccgt
agaaaagatcaaaggatcttcttgagatcctttttttctgcgcgtaatct
gctgcttgcaaacaaaaaaaccaccgctaccagcggtggtttgtttgccg
gatcaagagctaccaactctttttccgaaggtaactggcttcagcagagc
gcagataccaaatactgtccttctagtgtagccgtagttaggccaccact
tcaagaactctgtagcaccgcctacatacctcgctctgctaatcctgtta
ccagtggctgctgccagtggcgataagtcgtgtcttaccgggttggactc
aagacgatagttaccggataaggcgcagcggtcgggctgaacggggggtt
cgtgcacacagcccagcttggagcgaacgacctacaccgaactgagatac
ctacagcgtgagctatgagaaagcgccacgcttcccgaagggagaaaggc
ggacaggtatccggtaagcggcagggtcggaacaggagagcgcacgaggg
agcttccagggggaaacgcctggtatctttatagtcctgtcgggtttcgc
cacctctgacttgagcgtcgatttttgtgatgctcgtcaggggggcggag
cctatggaaaaacgccagcaacgcggcctttttacggttcctggcctttt
gctgcggttttgctcacatgttctttcctgcgttatcccctgattctgtg
gataaccgtattaccgcctttgagtgagctgataccgctcgccgcagccg
aacgaccgagcgcagcgagtcagtgagcgaggaagcggaagagcgcccaa
tacgcaaaccgcctctccccgcgcgttggccgattcattaatgcagctgg
cacgacaggtttcccgactggaaagcgggcagtgagcgcaacgcaattaa
tgtgagttagctcactcattaggcaccccaggctttacactttatgcttc
cggctcgtatgttgtgtggaattgtgagcggataacaatttcacacagga
aacagctatgaccatgattacgccagatttaattaagg

SEQ ID NO:10

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGWFLHVTYVPAQEKNFTTAPAICHDGKAHFPREGVFVSNGTH
WFVTQRNFYEPQIITTDNTFVSGNCDVVIGIVNNTVYDPLQPELDSFKEE
LDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCG
SCCKFDEDDSEPVLKGVKLHYT

SEQ ID NO:11

ctgcgcgctcgctcgctcactgaggccgcccgggcaaagcccgggcgtcg
ggcgacctttggtcgcccggcctcagtgagcgagcgagcgcgcagagagg
gagtggttaagctagctagttattaatagtaatcaattacggggtcatta
gttcatagcccatatatggagttccgcgttacataacttacggtaaatgg
cccgcctggctgaccgcccaacgacccccgcccattgacgtcaataatga
cgtatgttcccatagtaacgccaatagggactttccattgacgtcaatgg
gtggagtatttacggtaaactgcccacttggcagtacatcaagtgtatca
tatgccaagtacgccccctattgacgtcaatgacggtaaatggcccgcct
ggcattatgcccagtacatgaccttatgggactttcctacttggcagtac
atctacgtattagtcatcgctattaccatggtgatgcggttttggcagta
catcaatgggcgtggatagcggtttgactcacggggatttccaagtctcc
accccattgacgtcaatgggagtttgttttggcaccaaaatcaacgggac
tttccaaaatgtcgtaacaactccgccccattgacgcaaatgggcggtag
gcgtgtacggtgggaggtctatataagcagagctggtttagtgaaccgtc
agatcctgcagaagttggtcgtgaggcactgggcaggtaagtatcaaggt
tacaagacaggtttaaggagaccaatagaaactgggcttgtcgagacaga
gaagactcttgcgtttctgataggcacctattggtcttactgacatccac
tttgcctttctctccacaggtgtccaggcggccgccatggatgcaatgaa
gagagggctctgctgtgtgctgctgctgtgtggagcagtcttcgtttcgg
ccagcGTGCAGCCAACCGAGTCTATCGTGCGCTTTCCTAATATCACAAAC
CTGTGCCCATTTGGCGAGGTGTTCAACGCAACCCGCTTCGCCAGCGTGTA
CGCCTGGAATAGGAAGCGGATCAGCAACTGCGTGGCCGACTATAGCGTGC
TGTACAACTCCGCCTCTTTCAGCACCTTTAAGTGCTATGGCGTGTCCCCC
ACAAAGCTGAATGACCTGTGCTTTACCAACGTCTACGCCGATTCTTTCGT
GATCAGGGGCGACGAGGTGCGCCAGATCGCCCCCGGCCAGACAGGCAAGA
TCGCAGACTACAATTATAAGCTGCCAGACGATTTCACCGGCTGCGTGATC
GCCTGGAACAGCAACAATCTGGATTCCAAAGTGGGCGGCAACTACAATTA
TCTGTACCGGCTGTTTAGAAAGAGCAATCTGAAGCCCTTCGAGAGGGACA
TCTCTACAGAAATCTACCAGGCCGGCAGCACCCCTTGCAATGGCGTGGAG
GGCTTTAACTGTTATTTCCCACTCCAGTCCTACGGCTTCCAGCCCACAAA
CGGCGTGGGCTATCAGCCTTACCGCGTGGTGGTGCTGAGCTTTGAGCTGC
TGCACGCCCCAGCAACAGTGTGCGGCCCCAAGAAGTCCACCAATCTGGTG
AAGAACAAGTGCGTGAACTTCtaataagcttggatccaatcaacctctgg
attacaaaatttgtgaaagattgactggtattcttaactatgttgctcct
tttacgctatgtggatacgctgctttaatgcctttgtatcatgctattgc
ttcccgtatggctttcattttctcctccttgtataaatcctggttagttc
ttgccacggcggaactcatcgccgcctgccttgcccgctgctggacaggg
gctcggctgttgggcactgacaattccgtggtgagatctgcctcgactgt
gccttctagttgccagccatctgttgtttgcccctcccccgtgccttcct
tgaccctggaaggtgccactcccactgtcctttcctaataaaatgaggaa
attgcatcgcattgtctgagtaggtgtcattctattctggggggtggggt
ggggcaggacagcaagggggaggattgggaagacaatagcaggcatgctg
gggactcgagttaagggcgaattcccgataaggatcttcctagagcatgg
ctacgtagataagtagcatggcgggttaatcattaactacaaggaacccc
tagtgatggagttggccactccctctctgcgcgctcgctcgctcactgag
gccgggcgaccaaaggtcgcccgacgcccgggctttgcccgggcggcctc
agtgagcgagcgagcgcgcagccttaattaacctaattcactggccgtcg
ttttacaacgtcgtgactgggaaaaccctggcgttacccaacttaatcgc
cttgcagcacatccccctttcgccagctggcgtaatagcgaagaggcccg
caccgatcgcccttcccaacagttgcgcagcctgaatggcgaatgggacg
cgccctgtagcggcgcattaagcgcggcgggtgtggtggttacgcgcagc
gtgaccgctacacttgccagcgccctagcgcccgctcctttcgctttctt
cccttcctttctcgccacgttcgccggctttccccgtcaagctctaaatc
gggggctccctttagggttccgatttagtgctttacggcacctcgacccc
aaaaaacttgattagggtgatggttcacgtagtgggccatcgccctgata
gacggtttttcgccctttgacgttggagtccacgttctttaatagtggac
tcttgttccaaactggaacaacactcaaccctatctcggtctattctttt
gatttataagggattttgccgatttcggcctattggttaaaaaatgagct
gatttaacaaaaatttaacgcgaattttaacaaaatattaacgtttataa
tttcaggtggcatctttcggggaaatgtgcgcggaacccctatttgttta
tttttctaaatacattcaaatatgtatccgctcatgagacaataaccctg
ataaatgcttcaataatattgaaaaaggaagagtATGAGCCATATTCAAC
GGGAAACGTCGAGGCCGCGATTAAATTCCAACATGGATGCTGATTTATAT
GGGTATAAATGGGCTCGCGATAATGTCGGGCAATCAGGTGCGACAATCTA
TCGCTTGTATGGGAAGCCCGATGCGCCAGAGTTGTTTCTGAAACATGGCA
AAGGTAGCGTTGCCAATGATGTTACAGATGAGATGGTCAGACTAAACTGG
CTGACGGAATTTATGCCTCTTCCGACCATCAAGCATTTTATCCGTACTCC
TGATGATGCATGGTTACTCACCACTGCGATCCCCGGAAAAACAGCATTCC
AGGTATTAGAAGAATATCCTGATTCAGGTGAAAATATTGTTGATGCGCTG
GCAGTGTTCCTGCGCCGGTTGCATTCGATTCCTGTTTGTAATTGTCCTTT
TAACAGCGATCGCGTATTTCGTCTCGCTCAGGCGCAATCACGAATGAATA
ACGGTTTGGTTGATGCGAGTGATTTTGATGACGAGCGTAATGGCTGGCCT
GTTGAACAAGTCTGGAAAGAAATGCATAAACTTTTGCCATTCTCACCGGA
TTCAGTCGTCACTCATGGTGATTTCTCACTTGATAACCTTATTTTTGACG
AGGGGAAATTAATAGGTTGTATTGATGTTGGACGAGTCGGAATCGCAGAC
CGATACCAGGATCTTGCCATCCTATGGAACTGCCTCGGTGAGTTTTCTCC
TTCATTACAGAAACGGCTTTTTCAAAAATATGGTATTGATAATCCTGATA
TGAATAAATTGCAGTTTCATTTGATGCTCGATGAGTTTTTCTAActgtca
gaccaagtttactcatatatactttagattgatttaaaacttcattttta
atttaaaaggatctaggtgaagatcctttttgataatctcatgaccaaaa
tcccttaacgtgagttttcgttccactgagcgtcagaccccgtagaaaag
atcaaaggatcttcttgagatcctttttttctgcgcgtaatctgctgctt
gcaaacaaaaaaaccaccgctaccagcggtggtttgtttgccggatcaag
agctaccaactctttttccgaaggtaactggcttcagcagagcgcagata
ccaaatactgtTcttctagtgtagccgtagttaggccaccacttcaagaa
ctctgtagcaccgcctacatacctcgctctgctaatcctgttaccagtgg
ctgctgccagtggcgataagtcgtgtcttaccgggttggactcaagacga
tagttaccggataaggcgcagcggtcgggctgaacggggggttcgtgcac
acagcccagcttggagcgaacgacctacaccgaactgagatacctacagc
gtgagctatgagaaagcgccacgcttcccgaagggagaaaggcggacagg
tatccggtaagcggcagggtcggaacaggagagcgcacgagggagcttcc
agggggaaacgcctggtatctttatagtcctgtcgggtttcgccacctct
gacttgagcgtcgatttttgtgatgctcgtcaggggggcggagcctatgg
aaaaacgccagcaacgcggcctttttacggttcctggccttttgctgGCC
ttttgctcacatgttctttcctgcgttatcccctgattctgtggataacc
gtattaccgcctttgagtgagctgataccgctcgccgcagccgaacgacc
gagcgcagcgagtcagtgagcgaggaagcggaagagcgcccaatacgcaa
accgcctctccccgcgcgttggccgattcattaatgcagctggcacgaca
ggtttcccgactggaaagcgggcagtgagcgcaacgcaattaatgtgagt
tagctcactcattaggcaccccaggctttacactttatgcttccggctcg
tatgttgtgtggaattgtgagcggataacaatttcacacaggaaacagct
atgaccatgattacgccagatttaattaaggGATCTGGGCCACTCCCTCT

SEQ ID NO:12

MDAMKRGLCCVLLLCGAVFVSASVQPTESIVRFPNITNLCPFGEVFNATR
FASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNF

SEQ ID NO:13

ccttaattaggctgcgcgctcgctcgctcactgaggccgcccgggcaaag
ccCGGGCGTCGGGCGACCTTTGGTCGCCCGGCCTCAGTGAGCGAGCGAGC
GCGCAGAGAGGGAGTGGCCAACTCCATCACTAGGGGTTCCTTGTAGTTAA
TGATTAACCCGCCATGCTACTTATCTACGTAGCCATGCTCTAGGAAGATC
GGAATTCGCCCTTAAGCTAGCTAGTTATTAATAGTAATCAATTACGGGGT
CATTAGTTCATAGCCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGAGTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACCATGGTGATGCGGTTTTGG
CAGTACATCAATGGGCGTGGATAGCGGTTTGACTCACGGGGATTTCCAAG
TCTCCACCCCATTGACGTCAATGGGAGTTTGTTTTGGCACCAAAATCAAC
GGGACTTTCCAAAATGTCGTAACAACTCCGCCCCATTGACGCAAATGGGC
GGTAGGCGTGTACGGTGGGAGGTCTATATAAGCAGAGCTGGTTTAGTGAA
CCGTCAGATCCTGCAGAAGTTGGTCGTGAGGCACTGGGCAGGTAAGTATC
AAGGTTACAAGACAGGTTTAAGGAGACCAATAGAAACTGGGCTTGTCGAG
ACAGAGAAGACTCTTGCGTTTCTGATAGGCACCTATTGGTCTTACTGACA
TCCACTTTGCCTTTCTCTCCACAGGTGTCCAGGCGGCCGCCATGGATGCA
ATGAAGAGAGGGCTCTGCTGTGTGCTGCTGCTGTGTGGAGCAGTCTTCGT
TTCGGCCAGCCAGTGCGTCAATCTGACAACTCGGACTCAGCTGCCACCTG
CTTATACTAATAGCTTCACCAGAGGCGTGTACTATCCTGACAAGGTGTTT
AGAAGCTCCGTGCTGCACTCTACACAGGATCTGTTTCTGCCATTCTTTAG
CAACGTGACCTGGTTCCACGCCATCCACGTGAGCGGCACCAATGGCACAA
AGCGGTTCGACAATCCCGTGCTGCCTTTTAACGATGGCGTGTACTTCGCC
TCTACCGAGAAGAGCAACATCATCAGAGGCTGGATCTTTGGCACCACACT
GGACTCCAAGACACAGTCTCTGCTGATCGTGAACAATGCCACCAACGTGG
TCATCAAGGTGTGCGAGTTCCAGTTTTGTAATGATCCCTTCCTGGGCGTG
TACTATCACAAGAACAATAAGAGCTGGATGGAGTCCGAGTTTAGAGTGTA
TTCTAGCGCCAACAACTGCACATTTGAGTACGTGAGCCAGCCTTTCCTGA
TGGACCTGGAGGGCAAGCAGGGCAATTTCAAGAACCTGAGGGAGTTCGTG
TTTAAGAATATCGACGGCTACTTCAAAATCTACTCTAAGCACACCCCCAT
CAACCTGGTGCGCGACCTGCCTCAGGGCTTCAGCGCCCTGGAGCCCCTGG
TGGATCTGCCTATCGGCATCAACATCACCCGGTTTCAGACACTGCTGGCC
CTGCACAGAAGCTACCTGACACCCGGCGACTCCTCTAGCGGATGGACCGC
CGGCGCTGCCGCCTACTATGTGGGCTACCTCCAGCCCCGGACCTTCCTGC
TGAAGTACAACGAGAATGGCACCATCACAGACGCAGTGGATTGCGCCCTG
GACCCCCTGAGCGAGACAAAGTGTACACTGAAGTCCTTTACCGTGGAGAA
GGGCATCTATCAGACATCCAATTTCAGGGTGCAGCCAACCGAGTCTATCG
TGCGCTTTCCTAATATCACAAACCTGTGCCCATTTGGCGAGGTGTTCAAC
GCAACCCGCTTCGCCAGCGTGTACGCCTGGAATAGGAAGCGGATCAGCAA
CTGCGTGGCCGACTATAGCGTGCTGTACAACTCCGCCTCTTTCAGCACCT
TTAAGTGCTATGGCGTGTCCCCCACAAAGCTGAATGACCTGTGCTTTACC
AACGTCTACGCCGATTCTTTCGTGATCAGGGGCGACGAGGTGCGCCAGAT
CGCCCCCGGCCAGACAGGCAAGATCGCAGACTACAATTATAAGCTGCCAG
ACGATTTCACCGGCTGCGTGATCGCCTGGAACAGCAACAATCTGGATTCC
AAAGTGGGCGGCAACTACAATTATCTGTACCGGCTGTTTAGAAAGAGCAA
TCTGAAGCCCTTCGAGAGGGACATCTCTACAGAAATCTACCAGGCCGGCA
GCACCCCTTGCAATGGCGTGGAGGGCTTTAACTGTTATTTCCCACTCCAG
TCCTACGGCTTCCAGCCCACAAACGGCGTGGGCTATCAGCCTTACCGCGT
GGTGGTGCTGAGCTTTGAGCTGCTGCACGCCCCAGCAACAGTGTGCGGCC
CCAAGAAGTCCACCAATCTGGTGAAGAACAAGTGCGTGAACTTCAACTTC
AACGGCCTGACCGGCACAGGCGTGCTGACCGAGTCCAACAAGAAGTTCCT
GCCATTTCAGCAGTTCGGCAGGGACATCGCAGATACCACAGACGCCGTGC
GCGACCCACAGACCCTGGAGATCCTGGACATCACACCCTGCTCTTTCGGC
GGCGTGAGCGTGATCACACCCGGCACCAATACAAGCAACCAGGTGGCCGT
GCTGTATCAGGACGTGAATTGTACCGAGGTGCCCGTGGCTATCCACGCCG
ATCAGCTGACCCCAACATGGCGGGTGTACAGCACCGGCTCCAACGTCTTC
CAGACAAGAGCCGGATGCCTGATCGGAGCAGAGCACGTGAACAATTCCTA
TGAGTGCGACATCCCAATCGGCGCCGGCATCTGTGCCTCTTACCAGACCC
AGACAAACTCTCCCAGAAGAGCCCGGTAATAAGCTTGGATCCAATCAACC
TCTGGATTACAAAATTTGTGAAAGATTGACTGGTATTCTTAACTATGTTG
CTCCTTTTACGCTATGTGGATACGCTGCTTTAATGCCTTTGTATCATGCT
ATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGG
TGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCAC
GGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTT
CCTTGGCTGCTCGCCTGTGTTGCCACCTGGATTCTGCGCGGGACGTCCTT
CTGCTACGTCCCTTCGGCCCTCAATCCAGCGGACCTTCCTTCCCGCGGCC
TGCTGCCGGCTCTGCGGCCTCTTCCGCGTCTTCGAGATCTGCCTCGACTG
TGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGA
AATTGCATCGCATTGTCTGAGTAGGTGTCATTCTATTCTGGGGGGTGGGG
TGGGGCAGGACAGCAAGGGGGAGGATTGGGAAGACAATAGCAGGCATGCT
GGGGACTCGAGTTAAGGGCGAATTCCCGATAAGGATCTTCCTAGAGCATG
GCTACGTAGATAAGTAGCATGGCGGGTTAATCATTAACTACAAGGAACCC
CTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCT
CAGTGAGCGAGCGAGCGCGCAGCCTTAATTAACCTAATTCACTGGCCGTC
GTTTTACAACGTCGTGACTGGGAAAACCCTGGCGTTACCCAACTTAATCG
CCTTGCAGCACATCCCCCTTTCGCCAGCTGGCGTAATAGCGAAGAGGCCC
GCACCGATCGCCCTTCCCAACAGTTGCGCAGCCTGAATGGCGAATGGGAC
GCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCT
TCCCTTCCTTTCTCGCCACGTTCGCCGGCTTTCCCCGTCAAGCTCTAAAT
CGGGGGCTCCCTTTAGGGTTCCGATTTAGTGCTTTACGGCACCTCGACCC
CAAAAAACTTGATTAGGGTGATGGTTCACGTAGTGGGCCATCGCCCTGAT
AGACGGTTTTTCGCCCTTTGACGTTGGAGTCCACGTTCTTTAATAGTGGA
CTCTTGTTCCAAACTGGAACAACACTCAACCCTATCTCGGTCTATTCTTT
TGATTTATAAGGGATTTTGCCGATTTCGGCCTATTGGTTAAAAAATGAGC
TGATTTAACAAAAATTTAACGCGAATTTTAACAAAATATTAACGTTTATA
ATTTCAGGTGGCATCTTTCGGGGAAATGTGCGCGGAACCCCTATTTGTTT
ATTTTTCTAAATACATTCAAATATGTATCCGCTCATGAGACAATAACCCT
GATAAATGCTTCAATAATATTGAAAAAGGAAGAGTATGAGCCATATTCAA
CGGGAAACGTCGAGGCCGCGATTAAATTCCAACATGGATGCTGATTTATA
TGGGTATAAATGGGCTCGCGATAATGTCGGGCAATCAGGTGCGACAATCT
ATCGCTTGTATGGGAAGCCCGATGCGCCAGAGTTGTTTCTGAAACATGGC
AAAGGTAGCGTTGCCAATGATGTTACAGATGAGATGGTCAGACTAAACTG
GCTGACGGAATTTATGCCTCTTCCGACCATCAAGCATTTTATCCGTACTC
CTGATGATGCATGGTTACTCACCACTGCGATCCCCGGAAAAACAGCATTC
CAGGTATTAGAAGAATATCCTGATTCAGGTGAAAATATTGTTGATGCGCT
GGCAGTGTTCCTGCGCCGGTTGCATTCGATTCCTGTTTGTAATTGTCCTT
TTAACAGCGATCGCGTATTTCGTCTCGCTCAGGCGCAATCACGAATGAAT
AACGGTTTGGTTGATGCGAGTGATTTTGATGACGAGCGTAATGGCTGGCC
TGTTGAACAAGTCTGGAAAGAAATGCATAAACTTTTGCCATTCTCACCGG
ATTCAGTCGTCACTCATGGTGATTTCTCACTTGATAACCTTATTTTTGAC
GAGGGGAAATTAATAGGTTGTATTGATGTTGGACGAGTCGGAATCGCAGA
CCGATACCAGGATCTTGCCATCCTATGGAACTGCCTCGGTGAGTTTTCTC
CTTCATTACAGAAACGGCTTTTTCAAAAATATGGTATTGATAATCCTGAT
ATGAATAAATTGCAGTTTCATTTGATGCTCGATGAGTTTTTCTAACTGTC
AGACCAAGTTTACTCATATATACTTTAGATTGATTTAAAACTTCATTTTT
AATTTAAAAGGATCTAGGTGAAGATCCTTTTTGATAATCTCATGACCAAA
ATCCCTTAACGTGAGTTTTCGTTCCACTGAGCGTCAGACCCCGTAGAAAA
GATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGAT
ACCAAATACTGTTCTTCTAGTGTAGCCGTAGTTAGGCCACCACTTCAAGA
ACTCTGTAGCACCGCCTACATACCTCGCTCTGCTAATCCTGTTACCAGTG
GCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCA
CACAGCCCAGCTTGGAGCGAACGACCTACACCGAACTGAGATACCTACAG
CGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTC
CAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGC
CTTTTGCTCACATGTTCTTTCCTGCGTTATCCCCTGATTCTGTGGATAAC
CGTATTACCGCCTTTGAGTGAGCTGATACCGCTCGCCGCAGCCGAACGAC
CGAGCGCAGCGAGTCAGTGAGCGAGGAAGCGGAAGAGCGCCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGCCAGATTTAATTAAGG

SEQ ID NO:14

MDAMKRGLCCVLLLCGAVFVSASQCVNLTTRTQLPPAYTNSFTRGVYYPD
KVFRSSVLHSTQDLFLPFFSNVTWFHAIHVSGTNGTKRFDNPVLPFNDGV
YFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLR
EFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVD
CALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPC
SFGGVSVITPGTNTSNQVAVLYQDVNCTEVPVAIHADQLTPTWRVYSTGS
NVFQTRAGCLIGAEHVNNSYECDIPIGAGICASYQTQTNSPRRAR

SEQ ID NO:15

cCTTAATTAGGCtgcgcgctcgctcgctcactgaggccgcccgggcaaag
cccgggcgtcgggcgacctttggtcgcccggcctcagtgagcgagcgagc
gcgcagagagggagtggccaactccatcactaggggttccttgtagttaa
tgattaacccgccatgctacttatctacgtagccatgctctaggaagatc
ggaattcgcccttaagctagctaggtcttgaaaggagtgggaattggctc
cggtgcccgtcagtgggcagagcgcacatcgcccacagtccccgagaagt
tggggggaggggtcggcaattgatccggtgcctagagaaggtggcgcggg
gtaaactgggaaagtgatgtcgtgtactggctccgcctttttcccgaggg
tgggggagaaccgtatataagtgcagtagtcgccgtgaacgttctttttc
gcaacgggtttgccgccagaacacagGCggccgcctagatgccaccaTGT
TCGTCTTCCTGGTCCTGCTGCCTCTGGTCTCCTCACAGTGCGTCAATtTc
ACAACTCGGACTCAGCTGCCACCTGCTTATACTAATAGCTTCACCAGAGG
CGTGTACTATCCTGACAAGGTGTTTAGAAGCTCCGTGCTGCACTCTACAC
AGGATCTGTTTCTGCCATTCTTTAGCAACGTGACCTGGTTCCACGCCATC
CACGTGAGCGGCACCAATGGCACAAAGCGGTTCGcCAATCCCGTGCTGCC
TTTTAACGATGGCGTGTACTTCGCCTCTACCGAGAAGAGCAACATCATCA
GAGGCTGGATCTTTGGCACCACACTGGACTCCAAGACACAGTCTCTGCTG
ATCGTGAACAATGCCACCAACGTGGTCATCAAGGTGTGCGAGTTCCAGTT
TTGTAATGATCCCTTCCTGGGCGTGTACTATCACAAGAACAATAAGAGCT
GGATGGAGTCCGAGTTTAGAGTGTATTCTAGCGCCAACAACTGCACATTT
GAGTACGTGAGCCAGCCTTTCCTGATGGACCTGGAGGGCAAGCAGGGCAA
TTTCAAGAACCTGAGGGAGTTCGTGTTTAAGAATATCGACGGCTACTTCA
AAATCTACTCTAAGCACACCCCCATCAACCTGGTGCGCGgCCTGCCTCAG
GGCTTCAGCGCCCTGGAGCCCCTGGTGGATCTGCCTATCGGCATCAACAT
CACCCGGTTTCAGACACTGCTGGCCCTGCACAtAAGCTACCTGACACCCG
GCGACTCCTCTAGCGGATGGACCGCCGGCGCTGCCGCCTACTATGTGGGC
TACCTCCAGCCCCGGACCTTCCTGCTGAAGTACAACGAGAATGGCACCAT
CACAGACGCAGTGGATTGCGCCCTGGACCCCCTGAGCGAGACAAAGTGTA
CACTGAAGTCCTTTACCGTGGAGAAGGGCATCTATCAGACATCCAATTTC
AGGGTGCAGCCAACCGAGTCTATCGTGCGCTTTCCTAATATCACAAACCT
GTGCCCATTTGGCGAGGTGTTCAACGCAACCCGCTTCGCCAGCGTGTACG
CCTGGAATAGGAAGCGGATCAGCAACTGCGTGGCCGACTATAGCGTGCTG
TACAACTCCGCCTCTTTCAGCACCTTTAAGTGCTATGGCGTGTCCCCCAC
AAAGCTGAATGACCTGTGCTTTACCAACGTCTACGCCGATTCTTTCGTGA
TCAGGGGCGACGAGGTGCGCCAGATCGCCCCCGGCCAGACAGGCAAcATC
GCAGACTACAATTATAAGCTGCCAGACGATTTCACCGGCTGCGTGATCGC
CTGGAACAGCAACAATCTGGATTCCAAAGTGGGCGGCAACTACAATTATC
TGTACCGGCTGTTTAGAAAGAGCAATCTGAAGCCCTTCGAGAGGGACATC
TCTACAGAAATCTACCAGGCCGGCAGCACCCCTTGCAATGGCGTGaAGGG
CTTTAACTGTTATTTCCCACTCCAGTCCTACGGCTTCCAGCCCACAtACG
GCGTGGGCTATCAGCCTTACCGCGTGGTGGTGCTGAGCTTTGAGCTGCTG
CACGCCCCAGCAACAGTGTGCGGCCCCAAGAAGTCCACCAATCTGGTGAA
GAACAAGTGCGTGAACTTCAACTTCAACGGCCTGACCGGCACAGGCGTGC
TGACCGAGTCCAACAAGAAGTTCCTGCCATTTCAGCAGTTCGGCAGGGAC
ATCGCAGATACCACAGACGCCGTGCGCGACCCACAGACCCTGGAGATCCT
GGACATCACACCCTGCTCTTTCGGCGGCGTGAGCGTGATCACACCCGGCA
CCAATACAAGCAACCAGGTGgccgtgctgtatcagggggtgaattgtacc
gaggTGCCCGTGGCTATCCACGCCGATCAGCTGACCCCAACATGGCGGGT
GTACAGCACCGGCTCCAACGTCTTCCAGACAAGAGCCGGATGCCTGATCG
GAGCAGAGCACGTGAACAATTCCTATGAGTGCGACATCCCAATCGGCGCC
GGCATCTGTGCCTCTTACCAGACCcagacaaactctcccggaagcgccag
tagcgtggcctcccagTCTATCATCGCCTATACCATGTCCCTGGGCGtCG
AGAACAGCGTGGCCTACTCTAACAATAGCATCGCCATCCCAACCAACTTC
ACAATCTCTGTGACCACAGAGATCCTGCCCGTGTCCATGACCAAGACATC
TGTGGACTGCACAATGTATATCTGTGGCGATTCTACCGAGTGCAGCAACC
TGCTGCTCCAGTACGGCAGCTTTTGTACCCAGCTGAATAGAGCCCTGACA
GGCATCGCCGTGGAGCAGGATAAGAACACACAGGAGGTGTTCGCCCAGGT
GAAGCAAATCTACAAGACCCCCCCTATCAAGGACTTTGGCGGCTTCAATT
TTTCCCAGATCCTGCCTGATCCATCCAAGCCTTCTAAGCGGAGCTTTATC
GAGGACCTGCTGTTCAACAAGGTGACCCTGGCCGATGCCGGCTTCATCAA
GCAGTATGGCGATTGCCTGGGCGACATCGCAGCCAGGGACCTGATCTGCG
CCCAGAAGTTTAATGGCCTGACCGTGCTGCCACCCCTGCTGACAGATGAG
ATGATCGCACAGTACACAAGCGCCCTGCTGGCCGGCACCATCACATCCGG
ATGGACCTTCGGCGCAGGAGCCGCCCTCCAGATCCCCTTTGCCATGCAGA
TGGCCTATAGGTTCAACGGCATCGGCGTGACCCAGAATGTGCTGTACGAG
AACCAGAAGCTGATCGCCAATCAGTTTAACTCCGCCATCGGCAAGATCCA
GGACAGCCTGTCCTCTACAGCCAGCGCCCTGGGCAAGCTCCAGGATGTGG
TGAATCAGAACGCCCAGGCCCTGAATACCCTGGTGAAGCAGCTGAGCAGC
AACTTCGGCGCCATCTCTAGCGTGCTGAATGAcatcctgagccggctgga
cccaccggaggcagaggtgcagatcGACCGGCTGATCACCGGCCGGCTCC
AGAGCCTCCAGACCTATGTGACACAGCAGCTGATCAGGGCCGCCGAGATC
AGGGCCAGCGCCAATCTGGCAGCAACCAAGATGTCCGAGTGCGTGCTGGG
CCAGTCTAAGAGAGTGGACTTTTGTGGCAAGGGCTATCACCTGATGTCCT
TCCCTCAGTCTGCCCCACACGGCGTGGTGTTTCTGCACGTGACCTACGTG
CCCGCCCAGGAGAAGAACTTCACCACAGCCCCTGCCATCTGCCACGATGG
CAAGGCCCACTTTCCAAGGGAGGGCGTGTTCGTGTCCAACGGCACCCACT
GGTTTGTGACACAGCGCAATTTCTACGAGCCCCAGATCATCACCACAGAC
AACACCTTCGTGAGCGGCAACTGTGACGTGGTCATCGGCATCGTGAACAA
TACCGTGTATGATCCACTCCAGCCCGAGCTGGACAGCTTTAAGGAGGAGC
TGGATAAGTATTTCAAGAATCACACCTCCCCTGACGTGGATCTGGGCGAC
ATCAGCGGCATCAATGCCTCCGTGGTGAACATCCAGAAGGAGATCGACCG
CCTGAACGAGGTGGCTAAGAATCTGAACGAGAGCCTGATCGACCTCCAGG
AGCTGGGCAAGTATGAGCAGTACATCAAGTGGCCCTGGTACATCTGGCTG
GGCTTCATCGCCGGCCTGATCGCCATCGTGATGGTGACCATCATGCTGTG
CTGTATGACATCCTGCTGTTCTTGCCTGAAGGGCTGCTGTAGCTGTGGCT
CCTGCTGTAAGTTTGACGAGGATGACTCTGAACCTGTGCTGAAGGGCGTG
AAGCTGCACTACACCTAAATCGGATCTAATAAAATATCTTTATTTTCATT
ACATCTGTGTGTTGGTTTTTTGTGTGCtcgagttaagggcgaattcccga
taaggatcttcctagagcatggctacgtagataagtagcatggcgggtta
atcattaactacaaggaacccctagtgatggagttggccactccctctct
gcgcgctcgctcgctcactgaggccgggcgaccaaaggtcgcccgacgcc
cgggctttgcccgggcggcctcagtgagcgagcgagcgcgcagccttaat
taacctaattcactggccgtcgttttacaacgtcgtgactgggaaaaccc
tggcgttacccaacttaatcgccttgcagcacatccccctttcgccagct
ggcgtaatagcgaagaggcccgcaccgatcgcccttcccaacagttgcgc
agcctgaatggcgaatgggacgcgccctgtagcggcgcattaagcgcggc
gggtgtggtggttacgcgcagcgtgaccgctacacttgccagcgccctag
cgcccgctcctttcgctttcttcccttcctttctcgccacgttcgccggc
tttccccgtcaagctctaaatcgggggctccctttagggttccgatttag
tgctttacggcacctcgaccccaaaaaacttgattagggtgatggttcac
gtagtgggccatcgccctgatagacggtttttcgccctttgacgttggag
tccacgttctttaatagtggactcttgttccaaactggaacaacactcaa
ccctatctcggtctattcttttgatttataagggattttgccgatttcgg
cctattggttaaaaaatgagctgatttaacaaaaatttaacgcgaatttt
aacaaaatattaacgtttataatttcaggtggcatctttcggggaaatgt
gcgcggaacccctatttgtttatttttctaaatacattcaaatatgtatc
cgctcatgagacaataaccctgataaatgcttcaataatattgaaaaagg
aagagtATGAGCCATATTCAACGGGAAACGTCGAGGCCGCGATTAAATTC
CAACATGGATGCTGATTTATATGGGTATAAATGGGCTCGCGATAATGTCG
GGCAATCAGGTGCGACAATCTATCGCTTGTATGGGAAGCCCGATGCGCCA
GAGTTGTTTCTGAAACATGGCAAAGGTAGCGTTGCCAATGATGTTACAGA
TGAGATGGTCAGACTAAACTGGCTGACGGAATTTATGCCTCTTCCGACCA
TCAAGCATTTTATCCGTACTCCTGATGATGCATGGTTACTCACCACTGCG
ATCCCCGGAAAAACAGCATTCCAGGTATTAGAAGAATATCCTGATTCAGG
TGAAAATATTGTTGATGCGCTGGCAGTGTTCCTGCGCCGGTTGCATTCGA
TTCCTGTTTGTAATTGTCCTTTTAACAGCGATCGCGTATTTCGTCTCGCT
CAGGCGCAATCACGAATGAATAACGGTTTGGTTGATGCGAGTGATTTTGA
TGACGAGCGTAATGGCTGGCCTGTTGAACAAGTCTGGAAAGAAATGCATA
AACTTTTGCCATTCTCACCGGATTCAGTCGTCACTCATGGTGATTTCTCA
CTTGATAACCTTATTTTTGACGAGGGGAAATTAATAGGTTGTATTGATGT
TGGACGAGTCGGAATCGCAGACCGATACCAGGATCTTGCCATCCTATGGA
ACTGCCTCGGTGAGTTTTCTCCTTCATTACAGAAACGGCTTTTTCAAAAA
TATGGTATTGATAATCCTGATATGAATAAATTGCAGTTTCATTTGATGCT
CGATGAGTTTTTCTAActgtcagaccaagtttactcatatatactttaga
ttgatttaaaacttcatttttaatttaaaaggatctaggtgaagatcctt
tttgataatctcatgaccaaaatcccttaacgtgagttttcgttccactg
agcgtcagaccccgtagaaaagatcaaaggatcttcttgagatccttttt
ttctgcgcgtaatctgctgcttgcaaacaaaaaaaccaccgctaccagcg
gtggtttgtttgccggatcaagagctaccaactctttttccgaaggtaac
tggcttcagcagagcgcagataccaaatactgtccttctagtgtagccgt
agttaggccaccacttcaagaactctgtagcaccgcctacatacctcgct
ctgctaatcctgttaccagtggctgctgccagtggcgataagtcgtgtct
taccgggttggactcaagacgatagttaccggataaggcgcagcggtcgg
gctgaacggggggttcgtgcacacagcccagcttggagcgaacgacctac
accgaactgagatacctacagcgtgagctatgagaaagcgccacgcttcc
cgaagggagaaaggcggacaggtatccggtaagcggcagggtcggaacag
gagagcgcacgagggagcttccagggggaaacgcctggtatctttatagt
cctgtcgggtttcgccacctctgacttgagcgtcgatttttgtgatgctc
gtcaggggggcggagcctatggaaaaacgccagcaacgcggcctttttac
ggttcctggccttttgctgcggttttgctcacatgttctttcctgcgtta
tcccctgattctgtggataaccgtattaccgcctttgagtgagctgatac
cgctcgccgcagccgaacgaccgagcgcagcgagtcagtgagcgaggaag
cggaagagcgcccaatacgcaaaccgcctctccccgcgcgttggccgatt
cattaatgcagctggcacgacaggtttcccgactggaaagcgggcagtga
gcgcaacgcaattaatgtgagttagctcactcattaggcaccccaggctt
tacactttatgcttccggctcgtatgttgtgtggaattgtgagcggataa
caatttcacacaggaaacagctatgaccatgattacgccagatttaatta
agg

SEQ ID NO:16

cCTTAATTAGGCtgcgcgctcgctcgctcactgaggccgcccgggcaaag
cccgggcgtcgggcgacctttggtcgcccggcctcagtgagcgagcgagc
gcgcagagagggagtggccaactccatcactaggggttccttgtagttaa
tgattaacccgccatgctacttatctacgtagccatgctctaggaagatc
ggaattcgcccttaagctagctaggtcttgaaaggagtgggaattggctc
cggtgcccgtcagtgggcagagcgcacatcgcccacagtccccgagaagt
tggggggaggggtcggcaattgatccggtgcctagagaaggtggcgcggg
gtaaactgggaaagtgatgtcgtgtactggctccgcctttttcccgaggg
tgggggagaaccgtatataagtgcagtagtcgccgtgaacgttctttttc
gcaacgggtttgccgccagaacacagGCggccgcctagatgccaccaTGT
TCGTCTTCCTGGTCCTGCTGCCTCTGGTCTCCTCACAGTGCGTCAATtTc
ACAAaTCGGACTCAGCTGCCAagTGCTTATACTAATAGCTTCACCAGAGG
CGTGTACTATCCTGACAAGGTGTTTAGAAGCTCCGTGCTGCACTCTACAC
AGGATCTGTTTCTGCCATTCTTTAGCAACGTGACCTGGTTCCACGCCATC
CACGTGAGCGGCACCAATGGCACAAAGCGGTTCGACAATCCCGTGCTGCC
TTTTAACGATGGCGTGTACTTCGCCTCTACCGAGAAGAGCAACATCATCA
GAGGCTGGATCTTTGGCACCACACTGGACTCCAAGACACAGTCTCTGCTG
ATCGTGAACAATGCCACCAACGTGGTCATCAAGGTGTGCGAGTTCCAGTT
TTGTAATtATCCCTTCCTGGGCGTGTACTATCACAAGAACAATAAGAGCT
GGATGGAGTCCGAGTTTAGAGTGTATTCTAGCGCCAACAACTGCACATTT
GAGTACGTGAGCCAGCCTTTCCTGATGGACCTGGAGGGCAAGCAGGGCAA
TTTCAAGAACCTGAGtGAGTTCGTGTTTAAGAATATCGACGGCTACTTCA
AAATCTACTCTAAGCACACCCCCATCAACCTGGTGCGCGACCTGCCTCAG
GGCTTCAGCGCCCTGGAGCCCCTGGTGGATCTGCCTATCGGCATCAACAT
CACCCGGTTTCAGACACTGCTGGCCCTGCACAGAAGCTACCTGACACCCG
GCGACTCCTCTAGCGGATGGACCGCCGGCGCTGCCGCCTACTATGTGGGC
TACCTCCAGCCCCGGACCTTCCTGCTGAAGTACAACGAGAATGGCACCAT
CACAGACGCAGTGGATTGCGCCCTGGACCCCCTGAGCGAGACAAAGTGTA
CACTGAAGTCCTTTACCGTGGAGAAGGGCATCTATCAGACATCCAATTTC
AGGGTGCAGCCAACCGAGTCTATCGTGCGCTTTCCTAATATCACAAACCT
GTGCCCATTTGGCGAGGTGTTCAACGCAACCCGCTTCGCCAGCGTGTACG
CCTGGAATAGGAAGCGGATCAGCAACTGCGTGGCCGACTATAGCGTGCTG
TACAACTCCGCCTCTTTCAGCACCTTTAAGTGCTATGGCGTGTCCCCCAC
AAAGCTGAATGACCTGTGCTTTACCAACGTCTACGCCGATTCTTTCGTGA
TCAGGGGCGACGAGGTGCGCCAGATCGCCCCCGGCCAGACAGGCAcGATC
GCAGACTACAATTATAAGCTGCCAGACGATTTCACCGGCTGCGTGATCGC
CTGGAACAGCAACAATCTGGATTCCAAAGTGGGCGGCAACTACAATTATC
TGTACCGGCTGTTTAGAAAGAGCAATCTGAAGCCCTTCGAGAGGGACATC
TCTACAGAAATCTACCAGGCCGGCAGCACCCCTTGCAATGGCGTGaAGGG
CTTTAACTGTTATTTCCCACTCCAGTCCTACGGCTTCCAGCCCACAtACG
GCGTGGGCTATCAGCCTTACCGCGTGGTGGTGCTGAGCTTTGAGCTGCTG
CACGCCCCAGCAACAGTGTGCGGCCCCAAGAAGTCCACCAATCTGGTGAA
GAACAAGTGCGTGAACTTCAACTTCAACGGCCTGACCGGCACAGGCGTGC
TGACCGAGTCCAACAAGAAGTTCCTGCCATTTCAGCAGTTCGGCAGGGAC
ATCGCAGATACCACAGACGCCGTGCGCGACCCACAGACCCTGGAGATCCT
GGACATCACACCCTGCTCTTTCGGCGGCGTGAGCGTGATCACACCCGGCA
CCAATACAAGCAACCAGGTGgccgtgctgtatcagggggtgaattgtacc
gaggTGCCCGTGGCTATCCACGCCGATCAGCTGACCCCAACATGGCGGGT
GTACAGCACCGGCTCCAACGTCTTCCAGACAAGAGCCGGATGCCTGATCG
GAGCAGAGtACGTGAACAATTCCTATGAGTGCGACATCCCAATCGGCGCC
GGCATCTGTGCCTCTTACCAGACCcagacaaactctcccggaagcgccag
tagcgtggcctcccagTCTATCATCGCCTATACCATGTCCCTGGGCGCCG
AGAACAGCGTGGCCTACTCTAACAATAGCATCGCCATCCCAACCAACTTC
ACAATCTCTGTGACCACAGAGATCCTGCCCGTGTCCATGACCAAGACATC
TGTGGACTGCACAATGTATATCTGTGGCGATTCTACCGAGTGCAGCAACC
TGCTGCTCCAGTACGGCAGCTTTTGTACCCAGCTGAATAGAGCCCTGACA
GGCATCGCCGTGGAGCAGGATAAGAACACACAGGAGGTGTTCGCCCAGGT
GAAGCAAATCTACAAGACCCCCCCTATCAAGGACTTTGGCGGCTTCAATT
TTTCCCAGATCCTGCCTGATCCATCCAAGCCTTCTAAGCGGAGCTTTATC
GAGGACCTGCTGTTCAACAAGGTGACCCTGGCCGATGCCGGCTTCATCAA
GCAGTATGGCGATTGCCTGGGCGACATCGCAGCCAGGGACCTGATCTGCG
CCCAGAAGTTTAATGGCCTGACCGTGCTGCCACCCCTGCTGACAGATGAG
ATGATCGCACAGTACACAAGCGCCCTGCTGGCCGGCACCATCACATCCGG
ATGGACCTTCGGCGCAGGAGCCGCCCTCCAGATCCCCTTTGCCATGCAGA
TGGCCTATAGGTTCAACGGCATCGGCGTGACCCAGAATGTGCTGTACGAG
AACCAGAAGCTGATCGCCAATCAGTTTAACTCCGCCATCGGCAAGATCCA
GGACAGCCTGTCCTCTACAGCCAGCGCCCTGGGCAAGCTCCAGGATGTGG
TGAATCAGAACGCCCAGGCCCTGAATACCCTGGTGAAGCAGCTGAGCAGC
AACTTCGGCGCCATCTCTAGCGTGCTGAATGAcatcctgagccggctgga
cccaccggaggcagaggtgcagatcGACCGGCTGATCACCGGCCGGCTCC
AGAGCCTCCAGACCTATGTGACACAGCAGCTGATCAGGGCCGCCGAGATC
AGGGCCAGCGCCAATCTGGCAGCAAtCAAGATGTCCGAGTGCGTGCTGGG
CCAGTCTAAGAGAGTGGACTTTTGTGGCAAGGGCTATCACCTGATGTCCT
TCCCTCAGTCTGCCCCACACGGCGTGGTGTTTCTGCACGTGACCTACGTG
CCCGCCCAGGAGAAGAACTTCACCACAGCCCCTGCCATCTGCCACGATGG
CAAGGCCCACTTTCCAAGGGAGGGCGTGTTCGTGTCCAACGGCACCCACT
GGTTTGTGACACAGCGCAATTTCTACGAGCCCCAGATCATCACCACAGAC
AACACCTTCGTGAGCGGCAACTGTGACGTGGTCATCGGCATCGTGAACAA
TACCGTGTATGATCCACTCCAGCCCGAGCTGGACAGCTTTAAGGAGGAGC
TGGATAAGTATTTCAAGAATCACACCTCCCCTGACGTGGATCTGGGCGAC
ATCAGCGGCATCAATGCCTCCtTcGTGAACATCCAGAAGGAGATCGACCG
CCTGAACGAGGTGGCTAAGAATCTGAACGAGAGCCTGATCGACCTCCAGG
AGCTGGGCAAGTATGAGCAGTACATCAAGTGGCCCTGGTACATCTGGCTG
GGCTTCATCGCCGGCCTGATCGCCATCGTGATGGTGACCATCATGCTGTG
CTGTATGACATCCTGCTGTTCTTGCCTGAAGGGCTGCTGTAGCTGTGGCT
CCTGCTGTAAGTTTGACGAGGATGACTCTGAACCTGTGCTGAAGGGCGTG
AAGCTGCACTACACCTAAATCGGATCTAATAAAATATCTTTATTTTCATT
ACATCTGTGTGTTGGTTTTTTGTGTGCtcgagttaagggcgaattcccga
taaggatcttcctagagcatggctacgtagataagtagcatggcgggtta
atcattaactacaaggaacccctagtgatggagttggccactccctctct
gcgcgctcgctcgctcactgaggccgggcgaccaaaggtcgcccgacgcc
cgggctttgcccgggcggcctcagtgagcgagcgagcgcgcagccttaat
taacctaattcactggccgtcgttttacaacgtcgtgactgggaaaaccc
tggcgttacccaacttaatcgccttgcagcacatccccctttcgccagct
ggcgtaatagcgaagaggcccgcaccgatcgcccttcccaacagttgcgc
agcctgaatggcgaatgggacgcgccctgtagcggcgcattaagcgcggc
gggtgtggtggttacgcgcagcgtgaccgctacacttgccagcgccctag
cgcccgctcctttcgctttcttcccttcctttctcgccacgttcgccggc
tttccccgtcaagctctaaatcgggggctccctttagggttccgatttag
tgctttacggcacctcgaccccaaaaaacttgattagggtgatggttcac
gtagtgggccatcgccctgatagacggtttttcgccctttgacgttggag
tccacgttctttaatagtggactcttgttccaaactggaacaacactcaa
ccctatctcggtctattcttttgatttataagggattttgccgatttcgg
cctattggttaaaaaatgagctgatttaacaaaaatttaacgcgaatttt
aacaaaatattaacgtttataatttcaggtggcatctttcggggaaatgt
gcgcggaacccctatttgtttatttttctaaatacattcaaatatgtatc
cgctcatgagacaataaccctgataaatgcttcaataatattgaaaaagg
aagagtATGAGCCATATTCAACGGGAAACGTCGAGGCCGCGATTAAATTC
CAACATGGATGCTGATTTATATGGGTATAAATGGGCTCGCGATAATGTCG
GGCAATCAGGTGCGACAATCTATCGCTTGTATGGGAAGCCCGATGCGCCA
GAGTTGTTTCTGAAACATGGCAAAGGTAGCGTTGCCAATGATGTTACAGA
TGAGATGGTCAGACTAAACTGGCTGACGGAATTTATGCCTCTTCCGACCA
TCAAGCATTTTATCCGTACTCCTGATGATGCATGGTTACTCACCACTGCG
ATCCCCGGAAAAACAGCATTCCAGGTATTAGAAGAATATCCTGATTCAGG
TGAAAATATTGTTGATGCGCTGGCAGTGTTCCTGCGCCGGTTGCATTCGA
TTCCTGTTTGTAATTGTCCTTTTAACAGCGATCGCGTATTTCGTCTCGCT
CAGGCGCAATCACGAATGAATAACGGTTTGGTTGATGCGAGTGATTTTGA
TGACGAGCGTAATGGCTGGCCTGTTGAACAAGTCTGGAAAGAAATGCATA
AACTTTTGCCATTCTCACCGGATTCAGTCGTCACTCATGGTGATTTCTCA
CTTGATAACCTTATTTTTGACGAGGGGAAATTAATAGGTTGTATTGATGT
TGGACGAGTCGGAATCGCAGACCGATACCAGGATCTTGCCATCCTATGGA
ACTGCCTCGGTGAGTTTTCTCCTTCATTACAGAAACGGCTTTTTCAAAAA
TATGGTATTGATAATCCTGATATGAATAAATTGCAGTTTCATTTGATGCT
CGATGAGTTTTTCTAActgtcagaccaagtttactcatatatactttaga
ttgatttaaaacttcatttttaatttaaaaggatctaggtgaagatcctt
tttgataatctcatgaccaaaatcccttaacgtgagttttcgttccactg
agcgtcagaccccgtagaaaagatcaaaggatcttcttgagatccttttt
ttctgcgcgtaatctgctgcttgcaaacaaaaaaaccaccgctaccagcg
gtggtttgtttgccggatcaagagctaccaactctttttccgaaggtaac
tggcttcagcagagcgcagataccaaatactgtccttctagtgtagccgt
agttaggccaccacttcaagaactctgtagcaccgcctacatacctcgct
ctgctaatcctgttaccagtggctgctgccagtggcgataagtcgtgtct
taccgggttggactcaagacgatagttaccggataaggcgcagcggtcgg
gctgaacggggggttcgtgcacacagcccagcttggagcgaacgacctac
accgaactgagatacctacagcgtgagctatgagaaagcgccacgcttcc
cgaagggagaaaggcggacaggtatccggtaagcggcagggtcggaacag
gagagcgcacgagggagcttccagggggaaacgcctggtatctttatagt
cctgtcgggtttcgccacctctgacttgagcgtcgatttttgtgatgctc
gtcaggggggcggagcctatggaaaaacgccagcaacgcggcctttttac
ggttcctggccttttgctgcggttttgctcacatgttctttcctgcgtta
tcccctgattctgtggataaccgtattaccgcctttgagtgagctgatac
cgctcgccgcagccgaacgaccgagcgcagcgagtcagtgagcgaggaag
cggaagagcgcccaatacgcaaaccgcctctccccgcgcgttggccgatt
cattaatgcagctggcacgacaggtttcccgactggaaagcgggcagtga
gcgcaacgcaattaatgtgagttagctcactcattaggcaccccaggctt
tacactttatgcttccggctcgtatgttgtgtggaattgtgagcggataa
caatttcacacaggaaacagctatgaccatgattacgccagatttaatta
agg

SEQ ID NO:17

cCTTAATTAGGCtgcgcgctcgctcgctcactgaggccgcccgggcaaag
cccgggcgtcgggcgacctttggtcgcccggcctcagtgagcgagcgagc
gcgcagagagggagtggccaactccatcactaggggttccttgtagttaa
tgattaacccgccatgctacttatctacgtagccatgctctaggaagatc
ggaattcgcccttaagCTAGCcgttacataacttacggtaaatggcccgc
ctggctgaccgcccaacgacccccgcccattgacgtcaataatgacgtat
gttcccatagtaacgccaatagggactttccattgacgtcaatgggtgga
gtatttacggtaaactgcccacttggcagtacatcaagtgtatcatatgc
caagtacgccccctattgacgtcaatgacggtaaatggcccgcctggcat
tatgcccagtacatgaccttatgggactttcctacttggcagtacatcta
cgtattagtcatcgctattaccatgATACAGCTCCACCGCACATGCCACC
CTCCGGATATATTCGTCTCGAGCAAATCACTTGGCAGTCTAGCGGCggcc
gcctagatgccaccaTGTTCGTCTTCCTGGTCCTGCTGCCTCTGGTCTCC
TCACAGTGCGTCAATtTcACAACTCGGACTCAGCTGCCACCTGCTTATAC
TAATAGCTTCACCAGAGGCGTGTACTATCCTGACAAGGTGTTTAGAAGCT
CCGTGCTGCACTCTACACAGGATCTGTTTCTGCCATTCTTTAGCAACGTG
ACCTGGTTCCACGCCATCCACGTGAGCGGCACCAATGGCACAAAGCGGTT
CGcCAATCCCGTGCTGCCTTTTAACGATGGCGTGTACTTCGCCTCTACCG
AGAAGAGCAACATCATCAGAGGCTGGATCTTTGGCACCACACTGGACTCC
AAGACACAGTCTCTGCTGATCGTGAACAATGCCACCAACGTGGTCATCAA
GGTGTGCGAGTTCCAGTTTTGTAATGATCCCTTCCTGGGCGTGTACTATC
ACAAGAACAATAAGAGCTGGATGGAGTCCGAGTTTAGAGTGTATTCTAGC
GCCAACAACTGCACATTTGAGTACGTGAGCCAGCCTTTCCTGATGGACCT
GGAGGGCAAGCAGGGCAATTTCAAGAACCTGAGGGAGTTCGTGTTTAAGA
ATATCGACGGCTACTTCAAAATCTACTCTAAGCACACCCCCATCAACCTG
GTGCGCGgCCTGCCTCAGGGCTTCAGCGCCCTGGAGCCCCTGGTGGATCT
GCCTATCGGCATCAACATCACCCGGTTTCAGACACTGCTGGCCCTGCACA
tAAGCTACCTGACACCCGGCGACTCCTCTAGCGGATGGACCGCCGGCGCT
GCCGCCTACTATGTGGGCTACCTCCAGCCCCGGACCTTCCTGCTGAAGTA
CAACGAGAATGGCACCATCACAGACGCAGTGGATTGCGCCCTGGACCCCC
TGAGCGAGACAAAGTGTACACTGAAGTCCTTTACCGTGGAGAAGGGCATC
TATCAGACATCCAATTTCAGGGTGCAGCCAACCGAGTCTATCGTGCGCTT
TCCTAATATCACAAACCTGTGCCCATTTGGCGAGGTGTTCAACGCAACCC
GCTTCGCCAGCGTGTACGCCTGGAATAGGAAGCGGATCAGCAACTGCGTG
GCCGACTATAGCGTGCTGTACAACTCCGCCTCTTTCAGCACCTTTAAGTG
CTATGGCGTGTCCCCCACAAAGCTGAATGACCTGTGCTTTACCAACGTCT
ACGCCGATTCTTTCGTGATCAGGGGCGACGAGGTGCGCCAGATCGCCCCC
GGCCAGACAGGCAAcATCGCAGACTACAATTATAAGCTGCCAGACGATTT
CACCGGCTGCGTGATCGCCTGGAACAGCAACAATCTGGATTCCAAAGTGG
GCGGCAACTACAATTATCTGTACCGGCTGTTTAGAAAGAGCAATCTGAAG
CCCTTCGAGAGGGACATCTCTACAGAAATCTACCAGGCCGGCAGCACCCC
TTGCAATGGCGTGaAGGGCTTTAACTGTTATTTCCCACTCCAGTCCTACG
GCTTCCAGCCCACAtACGGCGTGGGCTATCAGCCTTACCGCGTGGTGGTG
CTGAGCTTTGAGCTGCTGCACGCCCCAGCAACAGTGTGCGGCCCCAAGAA
GTCCACCAATCTGGTGAAGAACAAGTGCGTGAACTTCAACTTCAACGGCC
TGACCGGCACAGGCGTGCTGACCGAGTCCAACAAGAAGTTCCTGCCATTT
CAGCAGTTCGGCAGGGACATCGCAGATACCACAGACGCCGTGCGCGACCC
ACAGACCCTGGAGATCCTGGACATCACACCCTGCTCTTTCGGCGGCGTGA
GCGTGATCACACCCGGCACCAATACAAGCAACCAGGTGgccgtgctgtat
cagggggtgaattgtaccgaggTGCCCGTGGCTATCCACGCCGATCAGCT
GACCCCAACATGGCGGGTGTACAGCACCGGCTCCAACGTCTTCCAGACAA
GAGCCGGATGCCTGATCGGAGCAGAGCACGTGAACAATTCCTATGAGTGC
GACATCCCAATCGGCGCCGGCATCTGTGCCTCTTACCAGACCcagacaaa
ctctcccggaagcgccagtagcgtggcctcccagTCTATCATCGCCTATA
CCATGTCCCTGGGCGtCGAGAACAGCGTGGCCTACTCTAACAATAGCATC
GCCATCCCAACCAACTTCACAATCTCTGTGACCACAGAGATCCTGCCCGT
GTCCATGACCAAGACATCTGTGGACTGCACAATGTATATCTGTGGCGATT
CTACCGAGTGCAGCAACCTGCTGCTCCAGTACGGCAGCTTTTGTACCCAG
CTGAATAGAGCCCTGACAGGCATCGCCGTGGAGCAGGATAAGAACACACA
GGAGGTGTTCGCCCAGGTGAAGCAAATCTACAAGACCCCCCCTATCAAGG
ACTTTGGCGGCTTCAATTTTTCCCAGATCCTGCCTGATCCATCCAAGCCT
TCTAAGCGGAGCTTTATCGAGGACCTGCTGTTCAACAAGGTGACCCTGGC
CGATGCCGGCTTCATCAAGCAGTATGGCGATTGCCTGGGCGACATCGCAG
CCAGGGACCTGATCTGCGCCCAGAAGTTTAATGGCCTGACCGTGCTGCCA
CCCCTGCTGACAGATGAGATGATCGCACAGTACACAAGCGCCCTGCTGGC
CGGCACCATCACATCCGGATGGACCTTCGGCGCAGGAGCCGCCCTCCAGA
TCCCCTTTGCCATGCAGATGGCCTATAGGTTCAACGGCATCGGCGTGACC
CAGAATGTGCTGTACGAGAACCAGAAGCTGATCGCCAATCAGTTTAACTC
CGCCATCGGCAAGATCCAGGACAGCCTGTCCTCTACAGCCAGCGCCCTGG
GCAAGCTCCAGGATGTGGTGAATCAGAACGCCCAGGCCCTGAATACCCTG
GTGAAGCAGCTGAGCAGCAACTTCGGCGCCATCTCTAGCGTGCTGAATGA
catcctgagccggctggacccaccggaggcagaggtgcagatcGACCGGC
TGATCACCGGCCGGCTCCAGAGCCTCCAGACCTATGTGACACAGCAGCTG
ATCAGGGCCGCCGAGATCAGGGCCAGCGCCAATCTGGCAGCAACCAAGAT
GTCCGAGTGCGTGCTGGGCCAGTCTAAGAGAGTGGACTTTTGTGGCAAGG
GCTATCACCTGATGTCCTTCCCTCAGTCTGCCCCACACGGCGTGGTGTTT
CTGCACGTGACCTACGTGCCCGCCCAGGAGAAGAACTTCACCACAGCCCC
TGCCATCTGCCACGATGGCAAGGCCCACTTTCCAAGGGAGGGCGTGTTCG
TGTCCAACGGCACCCACTGGTTTGTGACACAGCGCAATTTCTACGAGCCC
CAGATCATCACCACAGACAACACCTTCGTGAGCGGCAACTGTGACGTGGT
CATCGGCATCGTGAACAATACCGTGTATGATCCACTCCAGCCCGAGCTGG
ACAGCTTTAAGGAGGAGCTGGATAAGTATTTCAAGAATCACACCTCCCCT
GACGTGGATCTGGGCGACATCAGCGGCATCAATGCCTCCGTGGTGAACAT
CCAGAAGGAGATCGACCGCCTGAACGAGGTGGCTAAGAATCTGAACGAGA
GCCTGATCGACCTCCAGGAGCTGGGCAAGTATGAGCAGTACATCAAGTGG
CCCTGGTACATCTGGCTGGGCTTCATCGCCGGCCTGATCGCCATCGTGAT
GGTGACCATCATGCTGTGCTGTATGACATCCTGCTGTTCTTGCCTGAAGG
GCTGCTGTAGCTGTGGCTCCTGCTGTAAGTTTGACGAGGATGACTCTGAA
CCTGTGCTGAAGGGCGTGAAGCTGCACTACACCTAAATCGGATCTAATAA
AATATCTTTATTTTCATTACATCTGTGTGTTGGTTTTTTGTGTGCtcgag
ttaagggcgaattcccgataaggatcttcctagagcatggctacgtagat
aagtagcatggcgggttaatcattaactacaaggaacccctagtgatgga
gttggccactccctctctgcgcgctcgctcgctcactgaggccgggcgac
caaaggtcgcccgacgcccgggctttgcccgggcggcctcagtgagcgag
cgagcgcgcagccttaattaacctaattcactggccgtcgttttacaacg
tcgtgactgggaaaaccctggcgttacccaacttaatcgccttgcagcac
atccccctttcgccagctggcgtaatagcgaagaggcccgcaccgatcgc
ccttcccaacagttgcgcagcctgaatggcgaatgggacgcgccctgtag
cggcgcattaagcgcggcgggtgtggtggttacgcgcagcgtgaccgcta
cacttgccagcgccctagcgcccgctcctttcgctttcttcccttccttt
ctcgccacgttcgccggctttccccgtcaagctctaaatcgggggctccc
tttagggttccgatttagtgctttacggcacctcgaccccaaaaaacttg
attagggtgatggttcacgtagtgggccatcgccctgatagacggttttt
cgccctttgacgttggagtccacgttctttaatagtggactcttgttcca
aactggaacaacactcaaccctatctcggtctattcttttgatttataag
ggattttgccgatttcggcctattggttaaaaaatgagctgatttaacaa
aaatttaacgcgaattttaacaaaatattaacgtttataatttcaggtgg
catctttcggggaaatgtgcgcggaacccctatttgtttatttttctaaa
tacattcaaatatgtatccgctcatgagacaataaccctgataaatgctt
caataatattgaaaaaggaagagtATGAGCCATATTCAACGGGAAACGTC
GAGGCCGCGATTAAATTCCAACATGGATGCTGATTTATATGGGTATAAAT
GGGCTCGCGATAATGTCGGGCAATCAGGTGCGACAATCTATCGCTTGTAT
GGGAAGCCCGATGCGCCAGAGTTGTTTCTGAAACATGGCAAAGGTAGCGT
TGCCAATGATGTTACAGATGAGATGGTCAGACTAAACTGGCTGACGGAAT
TTATGCCTCTTCCGACCATCAAGCATTTTATCCGTACTCCTGATGATGCA
TGGTTACTCACCACTGCGATCCCCGGAAAAACAGCATTCCAGGTATTAGA
AGAATATCCTGATTCAGGTGAAAATATTGTTGATGCGCTGGCAGTGTTCC
TGCGCCGGTTGCATTCGATTCCTGTTTGTAATTGTCCTTTTAACAGCGAT
CGCGTATTTCGTCTCGCTCAGGCGCAATCACGAATGAATAACGGTTTGGT
TGATGCGAGTGATTTTGATGACGAGCGTAATGGCTGGCCTGTTGAACAAG
TCTGGAAAGAAATGCATAAACTTTTGCCATTCTCACCGGATTCAGTCGTC
ACTCATGGTGATTTCTCACTTGATAACCTTATTTTTGACGAGGGGAAATT
AATAGGTTGTATTGATGTTGGACGAGTCGGAATCGCAGACCGATACCAGG
ATCTTGCCATCCTATGGAACTGCCTCGGTGAGTTTTCTCCTTCATTACAG
AAACGGCTTTTTCAAAAATATGGTATTGATAATCCTGATATGAATAAATT
GCAGTTTCATTTGATGCTCGATGAGTTTTTCTAActgtcagaccaagttt
actcatatatactttagattgatttaaaacttcatttttaatttaaaagg
atctaggtgaagatcctttttgataatctcatgaccaaaatcccttaacg
tgagttttcgttccactgagcgtcagaccccgtagaaaagatcaaaggat
cttcttgagatcctttttttctgcgcgtaatctgctgcttgcaaacaaaa
aaaccaccgctaccagcggtggtttgtttgccggatcaagagctaccaac
tctttttccgaaggtaactggcttcagcagagcgcagataccaaatactg
tccttctagtgtagccgtagttaggccaccacttcaagaactctgtagca
ccgcctacatacctcgctctgctaatcctgttaccagtggctgctgccag
tggcgataagtcgtgtcttaccgggttggactcaagacgatagttaccgg
ataaggcgcagcggtcgggctgaacggggggttcgtgcacacagcccagc
ttggagcgaacgacctacaccgaactgagatacctacagcgtgagctatg
agaaagcgccacgcttcccgaagggagaaaggcggacaggtatccggtaa
gcggcagggtcggaacaggagagcgcacgagggagcttccagggggaaac
gcctggtatctttatagtcctgtcgggtttcgccacctctgacttgagcg
tcgatttttgtgatgctcgtcaggggggcggagcctatggaaaaacgcca
gcaacgcggcctttttacggttcctggccttttgctgcggttttgctcac
atgttctttcctgcgttatcccctgattctgtggataaccgtattaccgc
ctttgagtgagctgataccgctcgccgcagccgaacgaccgagcgcagcg
agtcagtgagcgaggaagcggaagagcgcccaatacgcaaaccgcctctc
cccgcgcgttggccgattcattaatgcagctggcacgacaggtttcccga
ctggaaagcgggcagtgagcgcaacgcaattaatgtgagttagctcactc
attaggcaccccaggctttacactttatgcttccggctcgtatgttgtgt
ggaattgtgagcggataacaatttcacacaggaaacagctatgaccatga
ttacgccagatttaattaagg

SEQ ID NO:18

cCTTAATTAGGCtgcgcgctcgctcgctcactgaggccgcccgggcaaag
cccgggcgtcgggcgacctttggtcgcccggcctcagtgagcgagcgagc
gcgcagagagggagtggccaactccatcactaggggttccttgtagttaa
tgattaacccgccatgctacttatctacgtagccatgctctaggaagatc
ggaattcgcccttaagCTAGCcgttacataacttacggtaaatggcccgc
ctggctgaccgcccaacgacccccgcccattgacgtcaataatgacgtat
gttcccatagtaacgccaatagggactttccattgacgtcaatgggtgga
gtatttacggtaaactgcccacttggcagtacatcaagtgtatcatatgc
caagtacgccccctattgacgtcaatgacggtaaatggcccgcctggcat
tatgcccagtacatgaccttatgggactttcctacttggcagtacatcta
cgtattagtcatcgctattaccatgATACAGCTCCACCGCACATGCCACC
CTCCGGATATATTCGTCTCGAGCAAATCACTTGGCAGTCTAGCGGCggcc
gcctagatgccaccaTGTTCGTCTTCCTGGTCCTGCTGCCTCTGGTCTCC
TCACAGTGCGTCAATtTcACAAaTCGGACTCAGCTGCCAagTGCTTATAC
TAATAGCTTCACCAGAGGCGTGTACTATCCTGACAAGGTGTTTAGAAGCT
CCGTGCTGCACTCTACACAGGATCTGTTTCTGCCATTCTTTAGCAACGTG
ACCTGGTTCCACGCCATCCACGTGAGCGGCACCAATGGCACAAAGCGGTT
CGACAATCCCGTGCTGCCTTTTAACGATGGCGTGTACTTCGCCTCTACCG
AGAAGAGCAACATCATCAGAGGCTGGATCTTTGGCACCACACTGGACTCC
AAGACACAGTCTCTGCTGATCGTGAACAATGCCACCAACGTGGTCATCAA
GGTGTGCGAGTTCCAGTTTTGTAATtATCCCTTCCTGGGCGTGTACTATC
ACAAGAACAATAAGAGCTGGATGGAGTCCGAGTTTAGAGTGTATTCTAGC
GCCAACAACTGCACATTTGAGTACGTGAGCCAGCCTTTCCTGATGGACCT
GGAGGGCAAGCAGGGCAATTTCAAGAACCTGAGtGAGTTCGTGTTTAAGA
ATATCGACGGCTACTTCAAAATCTACTCTAAGCACACCCCCATCAACCTG
GTGCGCGACCTGCCTCAGGGCTTCAGCGCCCTGGAGCCCCTGGTGGATCT
GCCTATCGGCATCAACATCACCCGGTTTCAGACACTGCTGGCCCTGCACA
GAAGCTACCTGACACCCGGCGACTCCTCTAGCGGATGGACCGCCGGCGCT
GCCGCCTACTATGTGGGCTACCTCCAGCCCCGGACCTTCCTGCTGAAGTA
CAACGAGAATGGCACCATCACAGACGCAGTGGATTGCGCCCTGGACCCCC
TGAGCGAGACAAAGTGTACACTGAAGTCCTTTACCGTGGAGAAGGGCATC
TATCAGACATCCAATTTCAGGGTGCAGCCAACCGAGTCTATCGTGCGCTT
TCCTAATATCACAAACCTGTGCCCATTTGGCGAGGTGTTCAACGCAACCC
GCTTCGCCAGCGTGTACGCCTGGAATAGGAAGCGGATCAGCAACTGCGTG
GCCGACTATAGCGTGCTGTACAACTCCGCCTCTTTCAGCACCTTTAAGTG
CTATGGCGTGTCCCCCACAAAGCTGAATGACCTGTGCTTTACCAACGTCT
ACGCCGATTCTTTCGTGATCAGGGGCGACGAGGTGCGCCAGATCGCCCCC
GGCCAGACAGGCAcGATCGCAGACTACAATTATAAGCTGCCAGACGATTT
CACCGGCTGCGTGATCGCCTGGAACAGCAACAATCTGGATTCCAAAGTGG
GCGGCAACTACAATTATCTGTACCGGCTGTTTAGAAAGAGCAATCTGAAG
CCCTTCGAGAGGGACATCTCTACAGAAATCTACCAGGCCGGCAGCACCCC
TTGCAATGGCGTGaAGGGCTTTAACTGTTATTTCCCACTCCAGTCCTACG
GCTTCCAGCCCACAtACGGCGTGGGCTATCAGCCTTACCGCGTGGTGGTG
CTGAGCTTTGAGCTGCTGCACGCCCCAGCAACAGTGTGCGGCCCCAAGAA
GTCCACCAATCTGGTGAAGAACAAGTGCGTGAACTTCAACTTCAACGGCC
TGACCGGCACAGGCGTGCTGACCGAGTCCAACAAGAAGTTCCTGCCATTT
CAGCAGTTCGGCAGGGACATCGCAGATACCACAGACGCCGTGCGCGACCC
ACAGACCCTGGAGATCCTGGACATCACACCCTGCTCTTTCGGCGGCGTGA
GCGTGATCACACCCGGCACCAATACAAGCAACCAGGTGgccgtgctgtat
cagggggtgaattgtaccgaggTGCCCGTGGCTATCCACGCCGATCAGCT
GACCCCAACATGGCGGGTGTACAGCACCGGCTCCAACGTCTTCCAGACAA
GAGCCGGATGCCTGATCGGAGCAGAGtACGTGAACAATTCCTATGAGTGC
GACATCCCAATCGGCGCCGGCATCTGTGCCTCTTACCAGACCcagacaaa
ctctcccggaagcgccagtagcgtggcctcccagTCTATCATCGCCTATA
CCATGTCCCTGGGCGCCGAGAACAGCGTGGCCTACTCTAACAATAGCATC
GCCATCCCAACCAACTTCACAATCTCTGTGACCACAGAGATCCTGCCCGT
GTCCATGACCAAGACATCTGTGGACTGCACAATGTATATCTGTGGCGATT
CTACCGAGTGCAGCAACCTGCTGCTCCAGTACGGCAGCTTTTGTACCCAG
CTGAATAGAGCCCTGACAGGCATCGCCGTGGAGCAGGATAAGAACACACA
GGAGGTGTTCGCCCAGGTGAAGCAAATCTACAAGACCCCCCCTATCAAGG
ACTTTGGCGGCTTCAATTTTTCCCAGATCCTGCCTGATCCATCCAAGCCT
TCTAAGCGGAGCTTTATCGAGGACCTGCTGTTCAACAAGGTGACCCTGGC
CGATGCCGGCTTCATCAAGCAGTATGGCGATTGCCTGGGCGACATCGCAG
CCAGGGACCTGATCTGCGCCCAGAAGTTTAATGGCCTGACCGTGCTGCCA
CCCCTGCTGACAGATGAGATGATCGCACAGTACACAAGCGCCCTGCTGGC
CGGCACCATCACATCCGGATGGACCTTCGGCGCAGGAGCCGCCCTCCAGA
TCCCCTTTGCCATGCAGATGGCCTATAGGTTCAACGGCATCGGCGTGACC
CAGAATGTGCTGTACGAGAACCAGAAGCTGATCGCCAATCAGTTTAACTC
CGCCATCGGCAAGATCCAGGACAGCCTGTCCTCTACAGCCAGCGCCCTGG
GCAAGCTCCAGGATGTGGTGAATCAGAACGCCCAGGCCCTGAATACCCTG
GTGAAGCAGCTGAGCAGCAACTTCGGCGCCATCTCTAGCGTGCTGAATGA
catcctgagccggctggacccaccggaggcagaggtgcagatcGACCGGC
TGATCACCGGCCGGCTCCAGAGCCTCCAGACCTATGTGACACAGCAGCTG
ATCAGGGCCGCCGAGATCAGGGCCAGCGCCAATCTGGCAGCAAtCAAGAT
GTCCGAGTGCGTGCTGGGCCAGTCTAAGAGAGTGGACTTTTGTGGCAAGG
GCTATCACCTGATGTCCTTCCCTCAGTCTGCCCCACACGGCGTGGTGTTT
CTGCACGTGACCTACGTGCCCGCCCAGGAGAAGAACTTCACCACAGCCCC
TGCCATCTGCCACGATGGCAAGGCCCACTTTCCAAGGGAGGGCGTGTTCG
TGTCCAACGGCACCCACTGGTTTGTGACACAGCGCAATTTCTACGAGCCC
CAGATCATCACCACAGACAACACCTTCGTGAGCGGCAACTGTGACGTGGT
CATCGGCATCGTGAACAATACCGTGTATGATCCACTCCAGCCCGAGCTGG
ACAGCTTTAAGGAGGAGCTGGATAAGTATTTCAAGAATCACACCTCCCCT
GACGTGGATCTGGGCGACATCAGCGGCATCAATGCCTCCtTcGTGAACAT
CCAGAAGGAGATCGACCGCCTGAACGAGGTGGCTAAGAATCTGAACGAGA
GCCTGATCGACCTCCAGGAGCTGGGCAAGTATGAGCAGTACATCAAGTGG
CCCTGGTACATCTGGCTGGGCTTCATCGCCGGCCTGATCGCCATCGTGAT
GGTGACCATCATGCTGTGCTGTATGACATCCTGCTGTTCTTGCCTGAAGG
GCTGCTGTAGCTGTGGCTCCTGCTGTAAGTTTGACGAGGATGACTCTGAA
CCTGTGCTGAAGGGCGTGAAGCTGCACTACACCTAAATCGGATCTAATAA
AATATCTTTATTTTCATTACATCTGTGTGTTGGTTTTTTGTGTGCtcgag
ttaagggcgaattcccgataaggatcttcctagagcatggctacgtagat
aagtagcatggcgggttaatcattaactacaaggaacccctagtgatgga
gttggccactccctctctgcgcgctcgctcgctcactgaggccgggcgac
caaaggtcgcccgacgcccgggctttgcccgggcggcctcagtgagcgag
cgagcgcgcagccttaattaacctaattcactggccgtcgttttacaacg
tcgtgactgggaaaaccctggcgttacccaacttaatcgccttgcagcac
atccccctttcgccagctggcgtaatagcgaagaggcccgcaccgatcgc
ccttcccaacagttgcgcagcctgaatggcgaatgggacgcgccctgtag
cggcgcattaagcgcggcgggtgtggtggttacgcgcagcgtgaccgcta
cacttgccagcgccctagcgcccgctcctttcgctttcttcccttccttt
ctcgccacgttcgccggctttccccgtcaagctctaaatcgggggctccc
tttagggttccgatttagtgctttacggcacctcgaccccaaaaaacttg
attagggtgatggttcacgtagtgggccatcgccctgatagacggttttt
cgccctttgacgttggagtccacgttctttaatagtggactcttgttcca
aactggaacaacactcaaccctatctcggtctattcttttgatttataag
ggattttgccgatttcggcctattggttaaaaaatgagctgatttaacaa
aaatttaacgcgaattttaacaaaatattaacgtttataatttcaggtgg
catctttcggggaaatgtgcgcggaacccctatttgtttatttttctaaa
tacattcaaatatgtatccgctcatgagacaataaccctgataaatgctt
caataatattgaaaaaggaagagtATGAGCCATATTCAACGGGAAACGTC
GAGGCCGCGATTAAATTCCAACATGGATGCTGATTTATATGGGTATAAAT
GGGCTCGCGATAATGTCGGGCAATCAGGTGCGACAATCTATCGCTTGTAT
GGGAAGCCCGATGCGCCAGAGTTGTTTCTGAAACATGGCAAAGGTAGCGT
TGCCAATGATGTTACAGATGAGATGGTCAGACTAAACTGGCTGACGGAAT
TTATGCCTCTTCCGACCATCAAGCATTTTATCCGTACTCCTGATGATGCA
TGGTTACTCACCACTGCGATCCCCGGAAAAACAGCATTCCAGGTATTAGA
AGAATATCCTGATTCAGGTGAAAATATTGTTGATGCGCTGGCAGTGTTCC
TGCGCCGGTTGCATTCGATTCCTGTTTGTAATTGTCCTTTTAACAGCGAT
CGCGTATTTCGTCTCGCTCAGGCGCAATCACGAATGAATAACGGTTTGGT
TGATGCGAGTGATTTTGATGACGAGCGTAATGGCTGGCCTGTTGAACAAG
TCTGGAAAGAAATGCATAAACTTTTGCCATTCTCACCGGATTCAGTCGTC
ACTCATGGTGATTTCTCACTTGATAACCTTATTTTTGACGAGGGGAAATT
AATAGGTTGTATTGATGTTGGACGAGTCGGAATCGCAGACCGATACCAGG
ATCTTGCCATCCTATGGAACTGCCTCGGTGAGTTTTCTCCTTCATTACAG
AAACGGCTTTTTCAAAAATATGGTATTGATAATCCTGATATGAATAAATT
GCAGTTTCATTTGATGCTCGATGAGTTTTTCTAActgtcagaccaagttt
actcatatatactttagattgatttaaaacttcatttttaatttaaaagg
atctaggtgaagatcctttttgataatctcatgaccaaaatcccttaacg
tgagttttcgttccactgagcgtcagaccccgtagaaaagatcaaaggat
cttcttgagatcctttttttctgcgcgtaatctgctgcttgcaaacaaaa
aaaccaccgctaccagcggtggtttgtttgccggatcaagagctaccaac
tctttttccgaaggtaactggcttcagcagagcgcagataccaaatactg
tccttctagtgtagccgtagttaggccaccacttcaagaactctgtagca
ccgcctacatacctcgctctgctaatcctgttaccagtggctgctgccag
tggcgataagtcgtgtcttaccgggttggactcaagacgatagttaccgg
ataaggcgcagcggtcgggctgaacggggggttcgtgcacacagcccagc
ttggagcgaacgacctacaccgaactgagatacctacagcgtgagctatg
agaaagcgccacgcttcccgaagggagaaaggcggacaggtatccggtaa
gcggcagggtcggaacaggagagcgcacgagggagcttccagggggaaac
gcctggtatctttatagtcctgtcgggtttcgccacctctgacttgagcg
tcgatttttgtgatgctcgtcaggggggcggagcctatggaaaaacgcca
gcaacgcggcctttttacggttcctggccttttgctgcggttttgctcac
atgttctttcctgcgttatcccctgattctgtggataaccgtattaccgc
ctttgagtgagctgataccgctcgccgcagccgaacgaccgagcgcagcg
agtcagtgagcgaggaagcggaagagcgcccaatacgcaaaccgcctctc
cccgcgcgttggccgattcattaatgcagctggcacgacaggtttcccga
ctggaaagcgggcagtgagcgcaacgcaattaatgtgagttagctcactc
attaggcaccccaggctttacactttatgcttccggctcgtatgttgtgt
ggaattgtgagcggataacaatttcacacaggaaacagctatgaccatga
ttacgccagatttaattaagg

SEQ ID NO:19

GTG CAG CCA ACC GAG

SEQ ID NO:20

ACA CCT CGC CAA ATG G

SEQ ID NO:21

TCT ATC GTG CGC TTT C

SEQ ID NO:22

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNF>NFNGLTGT
GVLTESNKKFLPFQQFGRDIADTTDAVRDPQTLEILDITPCSFGGVSVIT
PGTNTSNQVAVLYQDVNCTEVPVAIHADQLTPTWRVYSTGSNVFQTRAGC
LIGAEHVNNSYECDIPIGAGICASYQTQTNSPRRARSVASQSIIAYTMSL
GAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGG
FNFSQILPDPSKPSKRSFIEDLLFNKVTLADAGFIKQYGDCLGDIAARDL
ICAQKFNGLTVLPPLLTDEMIAQYTSALLAGTITSGWTFGAGAALQIPFA
MQMAYRFNGIGVTQNVLYENQKLIANQFNSAIGKIQDSLSSTASALGKLQ
DVVNQNAQALNTLVKQLSSNFGAISSVLNDILSRLDKVEAEVQIDRLITG
RLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLID
LQELGKYEQYIKWPWYIWLGFIAGLIAIVMVTIMLCCMTSCCSCLKGCCS
CGSCCKFDEDDSEPVLKGVKLHYT

SEQ ID NO:23

cCTTAATTAGGCtgcgcgctcgctcgctcactgaggccgcccgggcaaag
cccgggcgtcgggcgacctttggtcgcccggcctcagtgagcgagcgagc
gcgcagagagggagtggccaactccatcactaggggttccttgtagttaa
tgattaacccgccatgctacttatctacgtagccatgctctaggaagatc
ggaattcgcccttaagctagcGGTGTGGAAAGTCCCCAGGCTCCCCAGCA
GGCAGAAGTATGCAAAGCATGCATCTCAATTAGTCAGCAACCAGGTGTGG
AAAGTCCCCAGGCTCCCCAGCAGGCAGAAGTATGCAAAGCATGCATCTCA
ATTAGTCAGCAACCATAGTCCCGCCCCTAACTCCGCCCATCCCGCCCCTA
ACTCCGCCCAGTTCCGCCCATTCTCCGCCCCATGGCTGACTAATTTTTTT
TATTTATGCAGAGGCCGAGGCCGCCTCGGCCTCTGAGCTATTCCAGAAGT
AGTGAGGAGGCTTTTTTGGAGGCCTAGGCTTTTGCAAAgcggccgcctag
atgccaccaTGTTCGTCTTCCTGGTCCTGCTGCCTCTGGTCTCCTCACAG
TGCGTCAATCTGACAACTCGGACTCAGCTGCCACCTGCTTATACTAATAG
CTTCACCAGAGGCGTGTACTATCCTGACAAGGTGTTTAGAAGCTCCGTGC
TGCACTCTACACAGGATCTGTTTCTGCCATTCTTTAGCAACGTGACCTGG
TTCCACGCCATCCACGTGAGCGGCACCAATGGCACAAAGCGGTTCGACAA
TCCCGTGCTGCCTTTTAACGATGGCGTGTACTTCGCCTCTACCGAGAAGA
GCAACATCATCAGAGGCTGGATCTTTGGCACCACACTGGACTCCAAGACA
CAGTCTCTGCTGATCGTGAACAATGCCACCAACGTGGTCATCAAGGTGTG
CGAGTTCCAGTTTTGTAATGATCCCTTCCTGGGCGTGTACTATCACAAGA
ACAATAAGAGCTGGATGGAGTCCGAGTTTAGAGTGTATTCTAGCGCCAAC
AACTGCACATTTGAGTACGTGAGCCAGCCTTTCCTGATGGACCTGGAGGG
CAAGCAGGGCAATTTCAAGAACCTGAGGGAGTTCGTGTTTAAGAATATCG
ACGGCTACTTCAAAATCTACTCTAAGCACACCCCCATCAACCTGGTGCGC
GACCTGCCTCAGGGCTTCAGCGCCCTGGAGCCCCTGGTGGATCTGCCTAT
CGGCATCAACATCACCCGGTTTCAGACACTGCTGGCCCTGCACAGAAGCT
ACCTGACACCCGGCGACTCCTCTAGCGGATGGACCGCCGGCGCTGCCGCC
TACTATGTGGGCTACCTCCAGCCCCGGACCTTCCTGCTGAAGTACAACGA
GAATGGCACCATCACAGACGCAGTGGATTGCGCCCTGGACCCCCTGAGCG
AGACAAAGTGTACACTGAAGTCCTTTACCGTGGAGAAGGGCATCTATCAG
ACATCCAATTTCAGGGTGCAGCCAACCGAGTCTATCGTGCGCTTTCCTAA
TATCACAAACCTGTGCCCATTTGGCGAGGTGTTCAACGCAACCCGCTTCG
CCAGCGTGTACGCCTGGAATAGGAAGCGGATCAGCAACTGCGTGGCCGAC
TATAGCGTGCTGTACAACTCCGCCTCTTTCAGCACCTTTAAGTGCTATGG
CGTGTCCCCCACAAAGCTGAATGACCTGTGCTTTACCAACGTCTACGCCG
ATTCTTTCGTGATCAGGGGCGACGAGGTGCGCCAGATCGCCCCCGGCCAG
ACAGGCAAGATCGCAGACTACAATTATAAGCTGCCAGACGATTTCACCGG
CTGCGTGATCGCCTGGAACAGCAACAATCTGGATTCCAAAGTGGGCGGCA
ACTACAATTATCTGTACCGGCTGTTTAGAAAGAGCAATCTGAAGCCCTTC
GAGAGGGACATCTCTACAGAAATCTACCAGGCCGGCAGCACCCCTTGCAA
TGGCGTGGAGGGCTTTAACTGTTATTTCCCACTCCAGTCCTACGGCTTCC
AGCCCACAAACGGCGTGGGCTATCAGCCTTACCGCGTGGTGGTGCTGAGC
TTTGAGCTGCTGCACGCCCCAGCAACAGTGTGCGGCCCCAAGAAGTCCAC
CAATCTGGTGAAGAACAAGTGCGTGAACTTCAACTTCAACGGCCTGACCG
GCACAGGCGTGCTGACCGAGTCCAACAAGAAGTTCCTGCCATTTCAGCAG
TTCGGCAGGGACATCGCAGATACCACAGACGCCGTGCGCGACCCACAGAC
CCTGGAGATCCTGGACATCACACCCTGCTCTTTCGGCGGCGTGAGCGTGA
TCACACCCGGCACCAATACAAGCAACCAGGTGGCCGTGCTGTATCAGGAC
GTGAATTGTACCGAGGTGCCCGTGGCTATCCACGCCGATCAGCTGACCCC
AACATGGCGGGTGTACAGCACCGGCTCCAACGTCTTCCAGACAAGAGCCG
GATGCCTGATCGGAGCAGAGCACGTGAACAATTCCTATGAGTGCGACATC
CCAATCGGCGCCGGCATCTGTGCCTCTTACCAGACCcagacaaactctcc
cggaagcgccagtagcgtggcctcccagTCTATCATCGCCTATACCATGT
CCCTGGGCGCCGAGAACAGCGTGGCCTACTCTAACAATAGCATCGCCATC
CCAACCAACTTCACAATCTCTGTGACCACAGAGATCCTGCCCGTGTCCAT
GACCAAGACATCTGTGGACTGCACAATGTATATCTGTGGCGATTCTACCG
AGTGCAGCAACCTGCTGCTCCAGTACGGCAGCTTTTGTACCCAGCTGAAT
AGAGCCCTGACAGGCATCGCCGTGGAGCAGGATAAGAACACACAGGAGGT
GTTCGCCCAGGTGAAGCAAATCTACAAGACCCCCCCTATCAAGGACTTTG
GCGGCTTCAATTTTTCCCAGATCCTGCCTGATCCATCCAAGCCTTCTAAG
CGGAGCTTTATCGAGGACCTGCTGTTCAACAAGGTGACCCTGGCCGATGC
CGGCTTCATCAAGCAGTATGGCGATTGCCTGGGCGACATCGCAGCCAGGG
ACCTGATCTGCGCCCAGAAGTTTAATGGCCTGACCGTGCTGCCACCCCTG
CTGACAGATGAGATGATCGCACAGTACACAAGCGCCCTGCTGGCCGGCAC
CATCACATCCGGATGGACCTTCGGCGCAGGAGCCGCCCTCCAGATCCCCT
TTGCCATGCAGATGGCCTATAGGTTCAACGGCATCGGCGTGACCCAGAAT
GTGCTGTACGAGAACCAGAAGCTGATCGCCAATCAGTTTAACTCCGCCAT
CGGCAAGATCCAGGACAGCCTGTCCTCTACAGCCAGCGCCCTGGGCAAGC
TCCAGGATGTGGTGAATCAGAACGCCCAGGCCCTGAATACCCTGGTGAAG
CAGCTGAGCAGCAACTTCGGCGCCATCTCTAGCGTGCTGAATGAcatcct
gagccggctggacccaccggaggcagaggtgcagatcGACCGGCTGATCA
CCGGCCGGCTCCAGAGCCTCCAGACCTATGTGACACAGCAGCTGATCAGG
GCCGCCGAGATCAGGGCCAGCGCCAATCTGGCAGCAACCAAGATGTCCGA
GTGCGTGCTGGGCCAGTCTAAGAGAGTGGACTTTTGTGGCAAGGGCTATC
ACCTGATGTCCTTCCCTCAGTCTGCCCCACACGGCGTGGTGTTTCTGCAC
GTGACCTACGTGCCCGCCCAGGAGAAGAACTTCACCACAGCCCCTGCCAT
CTGCCACGATGGCAAGGCCCACTTTCCAAGGGAGGGCGTGTTCGTGTCCA
ACGGCACCCACTGGTTTGTGACACAGCGCAATTTCTACGAGCCCCAGATC
ATCACCACAGACAACACCTTCGTGAGCGGCAACTGTGACGTGGTCATCGG
CATCGTGAACAATACCGTGTATGATCCACTCCAGCCCGAGCTGGACAGCT
TTAAGGAGGAGCTGGATAAGTATTTCAAGAATCACACCTCCCCTGACGTG
GATCTGGGCGACATCAGCGGCATCAATGCCTCCGTGGTGAACATCCAGAA
GGAGATCGACCGCCTGAACGAGGTGGCTAAGAATCTGAACGAGAGCCTGA
TCGACCTCCAGGAGCTGGGCAAGTATGAGCAGTACATCAAGTGGCCCTGG
TACATCTGGCTGGGCTTCATCGCCGGCCTGATCGCCATCGTGATGGTGAC
CATCATGCTGTGCTGTATGACATCCTGCTGTTCTTGCCTGAAGGGCTGCT
GTAGCTGTGGCTCCTGCTGTAAGTTTGACGAGGATGACTCTGAACCTGTG
CTGAAGGGCGTGAAGCTGCACTACACCTAAATCGgatcttaagatacatt
gatgagtttggacaaaccacaactagaatgcagtgaaaaaaatgctttat
ttgtgaaatttgtgatgctattgctttatttgtaaccattataagctgca
ataaacaagttctcgagttaagggcgaattcccgataaggatcttcctag
agcatggctacgtagataagtagcatggcgggttaatcattaactacaag
gaacccctagtgatggagttggccactccctctctgcgcgctcgctcgct
cactgaggccgggcgaccaaaggtcgcccgacgcccgggctttgcccggg
cggcctcagtgagcgagcgagcgcgcagccttaattaacctaattcactg
gccgtcgttttacaacgtcgtgactgggaaaaccctggcgttacccaact
taatcgccttgcagcacatccccctttcgccagctggcgtaatagcgaag
aggcccgcaccgatcgcccttcccaacagttgcgcagcctgaatggcgaa
tgggacgcgccctgtagcggcgcattaagcgcggcgggtgtggtggttac
gcgcagcgtgaccgctacacttgccagcgccctagcgcccgctcctttcg
ctttcttcccttcctttctcgccacgttcgccggctttccccgtcaagct
ctaaatcgggggctccctttagggttccgatttagtgctttacggcacct
cgaccccaaaaaacttgattagggtgatggttcacgtagtgggccatcgc
cctgatagacggtttttcgccctttgacgttggagtccacgttctttaat
agtggactcttgttccaaactggaacaacactcaaccctatctcggtcta
ttcttttgatttataagggattttgccgatttcggcctattggttaaaaa
atgagctgatttaacaaaaatttaacgcgaattttaacaaaatattaacg
tttataatttcaggtggcatctttcggggaaatgtgcgcggaacccctat
ttgtttatttttctaaatacattcaaatatgtatccgctcatgagacaat
aaccctgataaatgcttcaataatattgaaaaaggaagagtATGAGCCAT
ATTCAACGGGAAACGTCGAGGCCGCGATTAAATTCCAACATGGATGCTGA
TTTATATGGGTATAAATGGGCTCGCGATAATGTCGGGCAATCAGGTGCGA
CAATCTATCGCTTGTATGGGAAGCCCGATGCGCCAGAGTTGTTTCTGAAA
CATGGCAAAGGTAGCGTTGCCAATGATGTTACAGATGAGATGGTCAGACT
AAACTGGCTGACGGAATTTATGCCTCTTCCGACCATCAAGCATTTTATCC
GTACTCCTGATGATGCATGGTTACTCACCACTGCGATCCCCGGAAAAACA
GCATTCCAGGTATTAGAAGAATATCCTGATTCAGGTGAAAATATTGTTGA
TGCGCTGGCAGTGTTCCTGCGCCGGTTGCATTCGATTCCTGTTTGTAATT
GTCCTTTTAACAGCGATCGCGTATTTCGTCTCGCTCAGGCGCAATCACGA
ATGAATAACGGTTTGGTTGATGCGAGTGATTTTGATGACGAGCGTAATGG
CTGGCCTGTTGAACAAGTCTGGAAAGAAATGCATAAACTTTTGCCATTCT
CACCGGATTCAGTCGTCACTCATGGTGATTTCTCACTTGATAACCTTATT
TTTGACGAGGGGAAATTAATAGGTTGTATTGATGTTGGACGAGTCGGAAT
CGCAGACCGATACCAGGATCTTGCCATCCTATGGAACTGCCTCGGTGAGT
TTTCTCCTTCATTACAGAAACGGCTTTTTCAAAAATATGGTATTGATAAT
CCTGATATGAATAAATTGCAGTTTCATTTGATGCTCGATGAGTTTTTCTA
Actgtcagaccaagtttactcatatatactttagattgatttaaaacttc
atttttaatttaaaaggatctaggtgaagatcctttttgataatctcatg
accaaaatcccttaacgtgagttttcgttccactgagcgtcagaccccgt
agaaaagatcaaaggatcttcttgagatcctttttttctgcgcgtaatct
gctgcttgcaaacaaaaaaaccaccgctaccagcggtggtttgtttgccg
gatcaagagctaccaactctttttccgaaggtaactggcttcagcagagc
gcagataccaaatactgtTcttctagtgtagccgtagttaggccaccact
tcaagaactctgtagcaccgcctacatacctcgctctgctaatcctgtta
ccagtggctgctgccagtggcgataagtcgtgtcttaccgggttggactc
aagacgatagttaccggataaggcgcagcggtcgggctgaacggggggtt
cgtgcacacagcccagcttggagcgaacgacctacaccgaactgagatac
ctacagcgtgagctatgagaaagcgccacgcttcccgaagggagaaaggc
ggacaggtatccggtaagcggcagggtcggaacaggagagcgcacgaggg
agcttccagggggaaacgcctggtatctttatagtcctgtcgggtttcgc
cacctctgacttgagcgtcgatttttgtgatgctcgtcaggggggcggag
cctatggaaaaacgccagcaacgcggcctttttacggttcctggcctttt
gctgGCCttttgctcacatgttctttcctgcgttatcccctgattctgtg
gataaccgtattaccgcctttgagtgagctgataccgctcgccgcagccg
aacgaccgagcgcagcgagtcagtgagcgaggaagcggaagagcgcccaa
tacgcaaaccgcctctccccgcgcgttggccgattcattaatgcagctgg
cacgacaggtttcccgactggaaagcgggcagtgagcgcaacgcaattaa
tgtgagttagctcactcattaggcaccccaggctttacactttatgcttc
cggctcgtatgttgtgtggaattgtgagcggataacaatttcacacagga
aacagctatgaccatgattacgccagatttaattaagg

SEQ ID NO:24

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

SEQ ID NO:25

ctgcgcgctcgctcgctcactgaggccgcccgggcaaagcccgggcgtcg
ggcgacctttggtcgcccggcctcagtgagcgagcgagcgcgcagagagg
gagtggttaagctagctagttattaatagtaatcaattacggggtcatta
gttcatagcccatatatggagttccgcgttacataacttacggtaaatgg
cccgcctggctgaccgcccaacgacccccgcccattgacgtcaataatga
cgtatgttcccatagtaacgccaatagggactttccattgacgtcaatgg
gtggagtatttacggtaaactgcccacttggcagtacatcaagtgtatca
tatgccaagtacgccccctattgacgtcaatgacggtaaatggcccgcct
ggcattatgcccagtacatgaccttatgggactttcctacttggcagtac
atctacgtattagtcatcgctattaccatggtgatgcggttttggcagta
catcaatgggcgtggatagcggtttgactcacggggatttccaagtctcc
accccattgacgtcaatgggagtttgttttggcaccaaaatcaacgggac
tttccaaaatgtcgtaacaactccgccccattgacgcaaatgggcggtag
gcgtgtacggtgggaggtctatataagcagagctggtttagtgaaccgtc
agatcctgcagaagttggtcgtgaggcactgggcaggtaagtatcaaggt
tacaagacaggtttaaggagaccaatagaaactgggcttgtcgagacaga
gaagactcttgcgtttctgataggcacctattggtcttactgacatccac
tttgcctttctctccacaggtgtccaggcggccgccatggatgcaatgaa
gagagggctctgctgtgtgctgctgctgtgtggagcagtcttcgtttcgg
ccagcGTGCAGCCAACCGAGTCTATCGTGCGCTTTCCTAATATCACAAAC
CTGTGCCCATTTGGCGAGGTGTTCAACGCAACCCGCTTCGCCAGCGTGTA
CGCCTGGAATAGGAAGCGGATCAGCAACTGCGTGGCCGACTATAGCGTGC
TGTACAACTCCGCCTCTTTCAGCACCTTTAAGTGCTATGGCGTGTCCCCC
ACAAAGCTGAATGACCTGTGCTTTACCAACGTCTACGCCGATTCTTTCGT
GATCAGGGGCGACGAGGTGCGCCAGATCGCCCCCGGCCAGACAGGCAAGA
TCGCAGACTACAATTATAAGCTGCCAGACGATTTCACCGGCTGCGTGATC
GCCTGGAACAGCAACAATCTGGATTCCAAAGTGGGCGGCAACTACAATTA
TCTGTACCGGCTGTTTAGAAAGAGCAATCTGAAGCCCTTCGAGAGGGACA
TCTCTACAGAAATCTACCAGGCCGGCAGCACCCCTTGCAATGGCGTGGAG
GGCTTTAACTGTTATTTCCCACTCCAGTCCTACGGCTTCCAGCCCACAAA
CGGCGTGGGCTATCAGCCTTACCGCGTGGTGGTGCTGAGCTTTGAGCTGC
TGCACGCCCCAGCAACAGTGTGCGGCCCCAAGAAGTCCACCAATCTGGTG
AAGAACAAGTGCGTGAACTTCtaataagcttggatccaatcaacctctgg
attacaaaatttgtgaaagattgactggtattcttaactatgttgctcct
tttacgctatgtggatacgctgctttaatgcctttgtatcatgctattgc
ttcccgtatggctttcattttctcctccttgtataaatcctggttagttc
ttgccacggcggaactcatcgccgcctgccttgcccgctgctggacaggg
gctcggctgttgggcactgacaattccgtggtgagatctgcctcgactgt
gccttctagttgccagccatctgttgtttgcccctcccccgtgccttcct
tgaccctggaaggtgccactcccactgtcctttcctaataaaatgaggaa
attgcatcgcattgtctgagtaggtgtcattctattctggggggtggggt
ggggcaggacagcaagggggaggattgggaagacaatagcaggcatgctg
gggactcgagttaagggcgaattcccgataaggatcttcctagagcatgg
ctacgtagataagtagcatggcgggttaatcattaactacaaggaacccc
tagtgatggagttggccactccctctctgcgcgctcgctcgctcactgag
gccgggcgaccaaaggtcgcccgacgcccgggctttgcccgggcggcctc
agtgagcgagcgagcgcgcagccttaattaacctaattcactggccgtcg
ttttacaacgtcgtgactgggaaaaccctggcgttacccaacttaatcgc
cttgcagcacatccccctttcgccagctggcgtaatagcgaagaggcccg
caccgatcgcccttcccaacagttgcgcagcctgaatggcgaatgggacg
cgccctgtagcggcgcattaagcgcggcgggtgtggtggttacgcgcagc
gtgaccgctacacttgccagcgccctagcgcccgctcctttcgctttctt
cccttcctttctcgccacgttcgccggctttccccgtcaagctctaaatc
gggggctccctttagggttccgatttagtgctttacggcacctcgacccc
aaaaaacttgattagggtgatggttcacgtagtgggccatcgccctgata
gacggtttttcgccctttgacgttggagtccacgttctttaatagtggac
tcttgttccaaactggaacaacactcaaccctatctcggtctattctttt
gatttataagggattttgccgatttcggcctattggttaaaaaatgagct
gatttaacaaaaatttaacgcgaattttaacaaaatattaacgtttataa
tttcaggtggcatctttcggggaaatgtgcgcggaacccctatttgttta
tttttctaaatacattcaaatatgtatccgctcatgagacaataaccctg
ataaatgcttcaataatattgaaaaaggaagagtATGAGCCATATTCAAC
GGGAAACGTCGAGGCCGCGATTAAATTCCAACATGGATGCTGATTTATAT
GGGTATAAATGGGCTCGCGATAATGTCGGGCAATCAGGTGCGACAATCTA
TCGCTTGTATGGGAAGCCCGATGCGCCAGAGTTGTTTCTGAAACATGGCA
AAGGTAGCGTTGCCAATGATGTTACAGATGAGATGGTCAGACTAAACTGG
CTGACGGAATTTATGCCTCTTCCGACCATCAAGCATTTTATCCGTACTCC
TGATGATGCATGGTTACTCACCACTGCGATCCCCGGAAAAACAGCATTCC
AGGTATTAGAAGAATATCCTGATTCAGGTGAAAATATTGTTGATGCGCTG
GCAGTGTTCCTGCGCCGGTTGCATTCGATTCCTGTTTGTAATTGTCCTTT
TAACAGCGATCGCGTATTTCGTCTCGCTCAGGCGCAATCACGAATGAATA
ACGGTTTGGTTGATGCGAGTGATTTTGATGACGAGCGTAATGGCTGGCCT
GTTGAACAAGTCTGGAAAGAAATGCATAAACTTTTGCCATTCTCACCGGA
TTCAGTCGTCACTCATGGTGATTTCTCACTTGATAACCTTATTTTTGACG
AGGGGAAATTAATAGGTTGTATTGATGTTGGACGAGTCGGAATCGCAGAC
CGATACCAGGATCTTGCCATCCTATGGAACTGCCTCGGTGAGTTTTCTCC
TTCATTACAGAAACGGCTTTTTCAAAAATATGGTATTGATAATCCTGATA
TGAATAAATTGCAGTTTCATTTGATGCTCGATGAGTTTTTCTAActgtca
gaccaagtttactcatatatactttagattgatttaaaacttcattttta
atttaaaaggatctaggtgaagatcctttttgataatctcatgaccaaaa
tcccttaacgtgagttttcgttccactgagcgtcagaccccgtagaaaag
atcaaaggatcttcttgagatcctttttttctgcgcgtaatctgctgctt
gcaaacaaaaaaaccaccgctaccagcggtggtttgtttgccggatcaag
agctaccaactctttttccgaaggtaactggcttcagcagagcgcagata
ccaaatactgtTcttctagtgtagccgtagttaggccaccacttcaagaa
ctctgtagcaccgcctacatacctcgctctgctaatcctgttaccagtgg
ctgctgccagtggcgataagtcgtgtcttaccgggttggactcaagacga
tagttaccggataaggcgcagcggtcgggctgaacggggggttcgtgcac
acagcccagcttggagcgaacgacctacaccgaactgagatacctacagc
gtgagctatgagaaagcgccacgcttcccgaagggagaaaggcggacagg
tatccggtaagcggcagggtcggaacaggagagcgcacgagggagcttcc
agggggaaacgcctggtatctttatagtcctgtcgggtttcgccacctct
gacttgagcgtcgatttttgtgatgctcgtcaggggggcggagcctatgg
aaaaacgccagcaacgcggcctttttacggttcctggccttttgctgGCC
ttttgctcacatgttctttcctgcgttatcccctgattctgtggataacc
gtattaccgcctttgagtgagctgataccgctcgccgcagccgaacgacc
gagcgcagcgagtcagtgagcgaggaagcggaagagcgcccaatacgcaa
accgcctctccccgcgcgttggccgattcattaatgcagctggcacgaca
ggtttcccgactggaaagcgggcagtgagcgcaacgcaattaatgtgagt
tagctcactcattaggcaccccaggctttacactttatgcttccggctcg
tatgttgtgtggaattgtgagcggataacaatttcacacaggaaacagct
atgaccatgattacgccagatttaattaaggGATCTGGGCCACTCCCTCT

SEQ ID NO:26

MDAMKRGLCCVLLLCGAVFVSASVQPTESIVRFPNITNLCPFGEVFNATR
FASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNF

SEQ ID NO:27

ccttaattaggctgcgcgctcgctcgctcactgaggccgcccgggcaaag
ccCGGGCGTCGGGCGACCTTTGGTCGCCCGGCCTCAGTGAGCGAGCGAGC
GCGCAGAGAGGGAGTGGCCAACTCCATCACTAGGGGTTCCTTGTAGTTAA
TGATTAACCCGCCATGCTACTTATCTACGTAGCCATGCTCTAGGAAGATC
GGAATTCGCCCTTAAGCTAGCTAGTTATTAATAGTAATCAATTACGGGGT
CATTAGTTCATAGCCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGAGTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACCATGGTGATGCGGTTTTGG
CAGTACATCAATGGGCGTGGATAGCGGTTTGACTCACGGGGATTTCCAAG
TCTCCACCCCATTGACGTCAATGGGAGTTTGTTTTGGCACCAAAATCAAC
GGGACTTTCCAAAATGTCGTAACAACTCCGCCCCATTGACGCAAATGGGC
GGTAGGCGTGTACGGTGGGAGGTCTATATAAGCAGAGCTGGTTTAGTGAA
CCGTCAGATCCTGCAGAAGTTGGTCGTGAGGCACTGGGCAGGTAAGTATC
AAGGTTACAAGACAGGTTTAAGGAGACCAATAGAAACTGGGCTTGTCGAG
ACAGAGAAGACTCTTGCGTTTCTGATAGGCACCTATTGGTCTTACTGACA
TCCACTTTGCCTTTCTCTCCACAGGTGTCCAGGCGGCCGCCATGGATGCA
ATGAAGAGAGGGCTCTGCTGTGTGCTGCTGCTGTGTGGAGCAGTCTTCGT
TTCGGCCAGCCAGTGCGTCAATCTGACAACTCGGACTCAGCTGCCACCTG
CTTATACTAATAGCTTCACCAGAGGCGTGTACTATCCTGACAAGGTGTTT
AGAAGCTCCGTGCTGCACTCTACACAGGATCTGTTTCTGCCATTCTTTAG
CAACGTGACCTGGTTCCACGCCATCCACGTGAGCGGCACCAATGGCACAA
AGCGGTTCGACAATCCCGTGCTGCCTTTTAACGATGGCGTGTACTTCGCC
TCTACCGAGAAGAGCAACATCATCAGAGGCTGGATCTTTGGCACCACACT
GGACTCCAAGACACAGTCTCTGCTGATCGTGAACAATGCCACCAACGTGG
TCATCAAGGTGTGCGAGTTCCAGTTTTGTAATGATCCCTTCCTGGGCGTG
TACTATCACAAGAACAATAAGAGCTGGATGGAGTCCGAGTTTAGAGTGTA
TTCTAGCGCCAACAACTGCACATTTGAGTACGTGAGCCAGCCTTTCCTGA
TGGACCTGGAGGGCAAGCAGGGCAATTTCAAGAACCTGAGGGAGTTCGTG
TTTAAGAATATCGACGGCTACTTCAAAATCTACTCTAAGCACACCCCCAT
CAACCTGGTGCGCGACCTGCCTCAGGGCTTCAGCGCCCTGGAGCCCCTGG
TGGATCTGCCTATCGGCATCAACATCACCCGGTTTCAGACACTGCTGGCC
CTGCACAGAAGCTACCTGACACCCGGCGACTCCTCTAGCGGATGGACCGC
CGGCGCTGCCGCCTACTATGTGGGCTACCTCCAGCCCCGGACCTTCCTGC
TGAAGTACAACGAGAATGGCACCATCACAGACGCAGTGGATTGCGCCCTG
GACCCCCTGAGCGAGACAAAGTGTACACTGAAGTCCTTTACCGTGGAGAA
GGGCATCTATCAGACATCCAATTTCAGGGTGCAGCCAACCGAGTCTATCG
TGCGCTTTCCTAATATCACAAACCTGTGCCCATTTGGCGAGGTGTTCAAC
GCAACCCGCTTCGCCAGCGTGTACGCCTGGAATAGGAAGCGGATCAGCAA
CTGCGTGGCCGACTATAGCGTGCTGTACAACTCCGCCTCTTTCAGCACCT
TTAAGTGCTATGGCGTGTCCCCCACAAAGCTGAATGACCTGTGCTTTACC
AACGTCTACGCCGATTCTTTCGTGATCAGGGGCGACGAGGTGCGCCAGAT
CGCCCCCGGCCAGACAGGCAAGATCGCAGACTACAATTATAAGCTGCCAG
ACGATTTCACCGGCTGCGTGATCGCCTGGAACAGCAACAATCTGGATTCC
AAAGTGGGCGGCAACTACAATTATCTGTACCGGCTGTTTAGAAAGAGCAA
TCTGAAGCCCTTCGAGAGGGACATCTCTACAGAAATCTACCAGGCCGGCA
GCACCCCTTGCAATGGCGTGGAGGGCTTTAACTGTTATTTCCCACTCCAG
TCCTACGGCTTCCAGCCCACAAACGGCGTGGGCTATCAGCCTTACCGCGT
GGTGGTGCTGAGCTTTGAGCTGCTGCACGCCCCAGCAACAGTGTGCGGCC
CCAAGAAGTCCACCAATCTGGTGAAGAACAAGTGCGTGAACTTCAACTTC
AACGGCCTGACCGGCACAGGCGTGCTGACCGAGTCCAACAAGAAGTTCCT
GCCATTTCAGCAGTTCGGCAGGGACATCGCAGATACCACAGACGCCGTGC
GCGACCCACAGACCCTGGAGATCCTGGACATCACACCCTGCTCTTTCGGC
GGCGTGAGCGTGATCACACCCGGCACCAATACAAGCAACCAGGTGGCCGT
GCTGTATCAGGACGTGAATTGTACCGAGGTGCCCGTGGCTATCCACGCCG
ATCAGCTGACCCCAACATGGCGGGTGTACAGCACCGGCTCCAACGTCTTC
CAGACAAGAGCCGGATGCCTGATCGGAGCAGAGCACGTGAACAATTCCTA
TGAGTGCGACATCCCAATCGGCGCCGGCATCTGTGCCTCTTACCAGACCC
AGACAAACTCTCCCAGAAGAGCCCGGTAATAAGCTTGGATCCAATCAACC
TCTGGATTACAAAATTTGTGAAAGATTGACTGGTATTCTTAACTATGTTG
CTCCTTTTACGCTATGTGGATACGCTGCTTTAATGCCTTTGTATCATGCT
ATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGG
TGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCAC
GGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTT
CCTTGGCTGCTCGCCTGTGTTGCCACCTGGATTCTGCGCGGGACGTCCTT
CTGCTACGTCCCTTCGGCCCTCAATCCAGCGGACCTTCCTTCCCGCGGCC
TGCTGCCGGCTCTGCGGCCTCTTCCGCGTCTTCGAGATCTGCCTCGACTG
TGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGA
AATTGCATCGCATTGTCTGAGTAGGTGTCATTCTATTCTGGGGGGTGGGG
TGGGGCAGGACAGCAAGGGGGAGGATTGGGAAGACAATAGCAGGCATGCT
GGGGACTCGAGTTAAGGGCGAATTCCCGATAAGGATCTTCCTAGAGCATG
GCTACGTAGATAAGTAGCATGGCGGGTTAATCATTAACTACAAGGAACCC
CTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCT
CAGTGAGCGAGCGAGCGCGCAGCCTTAATTAACCTAATTCACTGGCCGTC
GTTTTACAACGTCGTGACTGGGAAAACCCTGGCGTTACCCAACTTAATCG
CCTTGCAGCACATCCCCCTTTCGCCAGCTGGCGTAATAGCGAAGAGGCCC
GCACCGATCGCCCTTCCCAACAGTTGCGCAGCCTGAATGGCGAATGGGAC
GCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCT
TCCCTTCCTTTCTCGCCACGTTCGCCGGCTTTCCCCGTCAAGCTCTAAAT
CGGGGGCTCCCTTTAGGGTTCCGATTTAGTGCTTTACGGCACCTCGACCC
CAAAAAACTTGATTAGGGTGATGGTTCACGTAGTGGGCCATCGCCCTGAT
AGACGGTTTTTCGCCCTTTGACGTTGGAGTCCACGTTCTTTAATAGTGGA
CTCTTGTTCCAAACTGGAACAACACTCAACCCTATCTCGGTCTATTCTTT
TGATTTATAAGGGATTTTGCCGATTTCGGCCTATTGGTTAAAAAATGAGC
TGATTTAACAAAAATTTAACGCGAATTTTAACAAAATATTAACGTTTATA
ATTTCAGGTGGCATCTTTCGGGGAAATGTGCGCGGAACCCCTATTTGTTT
ATTTTTCTAAATACATTCAAATATGTATCCGCTCATGAGACAATAACCCT
GATAAATGCTTCAATAATATTGAAAAAGGAAGAGTATGAGCCATATTCAA
CGGGAAACGTCGAGGCCGCGATTAAATTCCAACATGGATGCTGATTTATA
TGGGTATAAATGGGCTCGCGATAATGTCGGGCAATCAGGTGCGACAATCT
ATCGCTTGTATGGGAAGCCCGATGCGCCAGAGTTGTTTCTGAAACATGGC
AAAGGTAGCGTTGCCAATGATGTTACAGATGAGATGGTCAGACTAAACTG
GCTGACGGAATTTATGCCTCTTCCGACCATCAAGCATTTTATCCGTACTC
CTGATGATGCATGGTTACTCACCACTGCGATCCCCGGAAAAACAGCATTC
CAGGTATTAGAAGAATATCCTGATTCAGGTGAAAATATTGTTGATGCGCT
GGCAGTGTTCCTGCGCCGGTTGCATTCGATTCCTGTTTGTAATTGTCCTT
TTAACAGCGATCGCGTATTTCGTCTCGCTCAGGCGCAATCACGAATGAAT
AACGGTTTGGTTGATGCGAGTGATTTTGATGACGAGCGTAATGGCTGGCC
TGTTGAACAAGTCTGGAAAGAAATGCATAAACTTTTGCCATTCTCACCGG
ATTCAGTCGTCACTCATGGTGATTTCTCACTTGATAACCTTATTTTTGAC
GAGGGGAAATTAATAGGTTGTATTGATGTTGGACGAGTCGGAATCGCAGA
CCGATACCAGGATCTTGCCATCCTATGGAACTGCCTCGGTGAGTTTTCTC
CTTCATTACAGAAACGGCTTTTTCAAAAATATGGTATTGATAATCCTGAT
ATGAATAAATTGCAGTTTCATTTGATGCTCGATGAGTTTTTCTAACTGTC
AGACCAAGTTTACTCATATATACTTTAGATTGATTTAAAACTTCATTTTT
AATTTAAAAGGATCTAGGTGAAGATCCTTTTTGATAATCTCATGACCAAA
ATCCCTTAACGTGAGTTTTCGTTCCACTGAGCGTCAGACCCCGTAGAAAA
GATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGAT
ACCAAATACTGTTCTTCTAGTGTAGCCGTAGTTAGGCCACCACTTCAAGA
ACTCTGTAGCACCGCCTACATACCTCGCTCTGCTAATCCTGTTACCAGTG
GCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCA
CACAGCCCAGCTTGGAGCGAACGACCTACACCGAACTGAGATACCTACAG
CGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTC
CAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGC
CTTTTGCTCACATGTTCTTTCCTGCGTTATCCCCTGATTCTGTGGATAAC
CGTATTACCGCCTTTGAGTGAGCTGATACCGCTCGCCGCAGCCGAACGAC
CGAGCGCAGCGAGTCAGTGAGCGAGGAAGCGGAAGAGCGCCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGCCAGATTTAATTAAGG

SEQ ID NO:28

MDAMKRGLCCVLLLCGAVFVSASQCVNLTTRTQLPPAYTNSFTRGVYYPD
KVFRSSVLHSTQDLFLPFFSNVTWFHAIHVSGTNGTKRFDNPVLPFNDGV
YFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLR
EFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVD
CALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPC
SFGGVSVITPGTNTSNQVAVLYQDVNCTEVPVAIHADQLTPTWRVYSTGS
NVFQTRAGCLIGAEHVNNSYECDIPIGAGICASYQTQTNSPRRAR

SEQ ID NO:29

ctagtttgcttgttaatcaataaaccgtttaattcgtttcagttgaactt
tggtctctgcgtatttctttcttatctagtttccatgctctagaggtcct
gtattagaggtcacgtgagtgttttgcgacattttgcgacaccatgtggt
cacgctgggtatttaagcccgagtgagcacgcagggtctccattttgaag
cgggaggtttgaacgcgcagccgccaagccgaattctgcagatatccatc
acactggcggccgctcgactagagcggccgccaccgcggtggagctccag
cttttgttcgcgcgcttggcgtaatcatggtcatagctgtttcctgtgtg
aaattccacagcctggggtgcctaattgcgttgcgctcactgcccgcttt
ccagtcgggaaacctgtcgtgccagctgcattaatgaatcggccaacgcg
cggggagaggcggtttgcgtattgggcgctcttccgcttcctcgctcact
gactcgctgcgctcggtcgttcggctgcggcgagcggtatcagctcactc
aaaggcggtaatacggttatccacagaatcaggggataacgcaggaaaga
acatgtgagcaaaaggccagcaaaaggccaggaaccgtaaaaaggccgcg
ttgctggcgtttttccataggctccgcccccctgacgagcatcacaaaaa
tcgacgctcaagtcagaggtggcgaaacccgacaggactataaagatacc
aggcgtttccccctggaagctccctcgtgcgctctcctgttccgaccctg
ccgcttaccggatacctgtccgcctttctcccttcgggaagcgtggcgct
ttctcatagctcacgctgtaggtatctcagttcggtgtaggtcgttcgct
ccaagctgggctgtgtgcacgaaccccccgttcagcccgaccgctgcgcc
ttatccggtaactatcgtcttgagtccaacccggtaagacacgacttatc
gccactggcagcagccactggtaacaggattagcagagcgaggtatgtag
gcggtgctacagagttcttgaagtggtggcctaactacggctacactaga
agaacagtatttggtatctgcgctctgctgaagccagttaccttcggaaa
aagagttggtagctcttgatccggcaaacaaaccaccgctggtagcggtg
gtttttttgtttgcaagcagcagattacgcgcagaaaaaaaggatctcaa
gaagatcctttgatcttttctacggggtctgacgctcagtggaacgaaaa
ctcacgttaagggattttggtcatgagattatcaaaaaggatcttcacct
agatccttttaaattaaaaatgaagttttaaatcaatctaaagtatatat
gagtaaacttggtctgacagaaataataaaaaagccggattaataatctg
gctttttatattctctctctagtatataaacgcagaaaggcccacccgaa
ggtgagccagtgtgactctagtattattagaaaaactcatcgagcatcaa
atgaaactgcaatttattcatatcaggattatcaataccatatttttgaa
aaagccgtttctgtaatgaaggagaaaactcaccgaggcagttccaaaga
atggcaaggtcctggtaacggtctgcgattccgacccgtccaacatcaat
acaacctattaatttcccctcgtcaaaaataaggttatcaagtgagaaat
caccatgagtgacgactgaatccggtgagaatggcaagagcttgtgcatt
tctttccagacttgttcaacaggccagccattacgctcgtcatcaaaatc
actcgcatcaaccaaaccgttattcatgcgtgattgcgcctgagcaagac
gaaatacacgatcgctgttaaaaggacaattacaaacaggaatcgaatgt
aaccggcgcaggaacacggccagcgcatcaacaatattttcacctgaatc
aggatattcttctaatacctggaaggctgttttcccaggaatcgcggtgg
tgagtaaccacgcatcatcaggagtacggataaaatgcttgatggtcggg
agaggcataaactccgtcagccagttgagacggaccatctcatctgtaac
atcattggcaacgctacctttgccatgtttcagaaacaactctggcgcat
cgggcttcccatacaagcgatagattgtcgcacctgattgcccgacatta
tcgcgagcccatttatacccatataaatcagcgtccatgttggagtttaa
gcgcggacgggagcaagacgtttcccgttgaatatggctcataacacccc
ttgtattactgtttatgtaagcagacagttttattgttcatgatgatata
tttttatcttgtgcaatgtaacatcagagattttgagacacaacgtggct
ttgttgaataaatcgaacttttgctgagttgaaggatcagctctagtaaa
ataataaaaaagccggattaataatctggctttttatattctctctctag
tatataaacgcagaaaggcccacccgaaggtgagccagtgtgacggcaca
tttccccgaaaagtgccacctaaattgtaagcgttaatattttgttaaaa
ttcgcgttaaatttttgttaaatcagctcattttttaaccaataggccga
aatcggcaaaatcccttataaatcaaaagaatagaccgagatagggttga
gtgttgttccagtttggaacaagagtccactattaaagaacgtggactcc
aacgtcaaagggcgaaaaaccgtctatcagggcgatggcccactacgtga
accatcaccctaatcaagttttttggggtcgaggtgccgtaaagcactaa
atcggaaccctaaagggagcccccgatttagagcttgacggggaaagccg
gcgaacgtggcgagaaaggaagggaagaaagcgaaaggagcgggcgctag
ggcgctggcaagtgtagcggtcacgctgcgcgtaaccaccacacccgccg
cgcttaatgcgccgctacagggcgcgtcccattcgccattcaggctgcgc
aactgttgggaagggcgatcggtgcgggcctcttcgctattacgccagct
ggcgaaagggggatgtgctgcaaggcgattaagttgggtaacgccagggt
tttcccagtcacgacgttgtaaaacgacggccagtgagcgcgcggcgaat
tgggtaccgggccccccctcgaggtcgacggtatcgtatttaagcccgag
tgagcacgcagggtctccattttgaagcgggaggtttgaacgcgcagccg
ccatgccggggttttacgagattgtgattaaggtccccagcgaccttgac
gagcatctgcccggcatttctgacagctttgtgaactgggtggccgagaa
ggaatgggagttgccgccagattctgacatggatctgaatctgattgagc
aggcacccctgaccgtggccgagaagctgcagcgcgactttctgacggaa
tggcgccgtgtgagtaaggccccggaggctcttttctttgtgcaatttga
gaagggagagagctacttccacatgcacgtgctcgtggaaaccaccgggg
tgaaatccatggttttgggacgtttcctgagtcagattcgcgaaaaactg
attcagagaatttaccgcgggatcgagccgactttgccaaactggttcgc
ggtcacaaagaccagaaatggcgccggaggcgggaacaaggtggtggatg
agtgctacatccccaattacttgctccccaaaacccagcctgagctccag
tgggcgtggactaatatggaacagtatttaagcgcctgtttgaatctcac
ggagcgtaaacggttggtggcgcagcatctgacgcacgtgtcgcagacgc
aggagcagaacaaagagaatcagaatcccaattctgatgcgccggtgatc
agatcaaaaacttcagccaggtacatggagctggtcgggtggctcgtgga
caaggggattacctcggagaagcagtggatccaggaggaccaggcctcat
acatctccttcaatgcggcctccaactcgcggtcccaaatcaaggctgcc
ttggacaatgcgggaaagattatgagcctgactaaaaccgcccccgacta
cctggtgggccagcagcccgtggaggacatttccagcaatcggatttata
aaattttggaactaaacgggtacgatccccaatatgcggcttccgtcttt
ctgggatgggccacgaaaaagttcggcaagaggaacaccatctggctgtt
tgggcctgcaactaccgggaagaccaacatcgcggaggccatagcccaca
ctgtgcccttctacgggtgcgtaaactggaccaatgagaactttcccttc
aacgactgtgtcgacaagatggtgatctggtgggaggaggggaagatgac
cgccaaggtcgtggagtcggccaaagccattctcggaggaagcaaggtgc
gcgtggaccagaaatgcaagtcctcggcccagatagacccgactcccgtg
atcgtcacctccaacaccaacatgtgcgccgtgattgacgggaactcaac
gaccttcgaacaccagcagccgttgcaagaccggatgttcaaatttgaac
tcacccgccgtctggatcatgactttgggaaggtcaccaagcaggaagtc
aaagactttttccggtgggcaaaggatcacgtggttgaggtggagcatga
attctacgtcaaaaagggtggagccaagaaaagacccgcccccagtgacg
cagatataagtgagcccaaacgggtgcgcgagtcagttgcgcagccatcg
acgtcagacgcggaAGCTTCGATCAACTACGCAGACAGGTACCAAAACAA
ATGTTCTCGTCACGTGGGCATGAATCTGATGCTGTTTCCCTGCAGACAAT
GCGAGAGAATGAATCAGAATTCAAATATCTGCTTCACTCACGGACAGAAA
GACTGTTTAGAGTGCTTTCCCGTGTCAGAATCTCAACCCGTTTCTGTCGT
CAAAAAGGCGTATCAGAAACTGTGCTACATTCATCATATCATGGGAAAGG
TGCCAGACGCTTGCACTGCCTGCGATCTGGTCAATGTGGATTTGGATGAC
TGCATCTTTGAACAATAAATGATTTAAATCAGGTATGGCTGCTGACGGTT
ATCTTCCAGATTGGCTCGAGGACAACCTCTCTGAGGGCATTCGCGAGTGG
TGGGACCTGAAACCTGGAGCCCCGAAGCCCAAGGCCAACCAGCAGAAGCA
GGACGACGGCCGGGGTCTGGTGCTTCCTGGCTACAAGTACCTCGGACCCT
TCAACGGACTCGACAAGGGGGAGCCCGTCAACGCGGCGGACGCAGCGGCC
CTCGAGCACGACAAGGCCTACGACCAGCAGCTCAAAGCGGGTGACAATCC
GTACCTGCGGTATAACCACGCCGACGCCGAGTTTCAGGAGCGTCTGCAAG
AAGATACGTCTTTTGGGGGCAACCTCGGGCGAGCAGTCTTCCAGGCCAAG
AAGAGGGTACTCGAACCTCTGGGCCTGGTTGAAGAAGGTGCTAAAACGGC
TCCTGGAAAGAAGAGACCGTTAGAGTCACCACAAGAGCCCGACTCCTCCT
CGGGCATCGGCAAAAAAGGCAAACAACCAGCCAGAAAGAGGCTCAACTTT
GAAGAGGACACTGGAGCCGGAGACGGACCCCCTGAAGGATCAGATACCAG
CGCCATGTCTTCAGACATTGAAATGCGTGCAGCACCGGGCGGAAATGCTG
TCGATGCGGGACAAGGTTCCGATGGAGTGGGTAATGCCTCGGGTGATTGG
CATTGCGATTCCACCTGGTCTGAGGGCAAGGTCACAACAACCTCGACCAG
AACCTGGGTCTTGCCCACCTACAACAACCACTTGTACCTGCGTCTCGGAA
CAACATCAAGCAGCAACACCTACAACGGATTCTCCACCCCCTGGGGATAT
TTTGACTTCAACAGATTCCACTGTCACTTCTCACCACGTGACTGGCAAAG
ACTCATCAACAACAACTGGGGACTACGACCAAAAGCCATGCGCGTTAAAA
TCTTCAATATCCAAGTTAAGGAGGTCACAACGTCGAACGGCGAGACTACG
GTCGCTAATAACCTTACCAGCACGGTTCAGATATTTGCGGACTCGTCGTA
TGAGCTCCCGTACGTGATGGACGCTGGACAAGAGGGGAGCCTGCCTCCTT
TCCCCAATGACGTGTTCATGGTGCCTCAATATGGCTACTGTGGCATCGTG
ACTGGCGAGAATCAGAACCAAACGGACAGAAACGCTTTCTACTGCCTGGA
GTATTTTCCTTCGCAAATGTTGAGAACTGGCAACAACTTTGAAATGGCTT
ACAACTTTGAGAAGGTGCCGTTCCACTCAATGTATGCTCACAGCCAGAGC
CTGGACAGACTGATGAATCCCCTCCTGGACCAGTACCTGTGGCACTTACA
GTCGACTACCTCTGGAGAGACTCTGAATCAAGGCAATGCAGCAACCACAT
TTGGAAAAATCAGGAGTGGAGACTTTGCCTTTTACAGAAAGAACTGGCTG
CCTGGGCCTTGTGTTAAACAGCAGAGATTCTCAAAAACTGCCAGTCAAAA
TTACAAGATTCCTGCCAGCGGGGGCAACGCTCTGTTAAAGTATGACACCC
ACTATACCTTAAACAACCGCTGGAGCAACATCGCGCCCGGACCTCCAATG
GCCACAGCCGGACCTTCGGATGGGGACTTCAGTAACGCCCAGCTTATATT
CCCTGGACCATCTGTTACCGGAAATACAACAACTTCAGCCAACAATCTGT
TGTTTACATCAGAAGAAGAAATTGCTGCCACCAACCCAAGAGACACGGAC
ATGTTTGGCCAGATTGCTGACAATAATCAGAATGCTACAACTGCTCCCAT
AACCGGCAACGTGACTGCTATGGGAGTGCTGCCTGGCATGGTGTGGCAAA
ACAGAGACATTTACTACCAAGGGCCAATTTGGGCCAAGATCCCACACGCG
GACGGACATTTTCATCCTTCACCGCTGATTGGTGGGTTTGGACTGAAACA
CCCGCCTCCCCAGATATTCATCAAGAACACTCCCGTACCTGCCAATCCTG
CGACAACCTTCACTGCAGCCAGAGTGGACTCTTTCATCACACAATACAGC
ACCGGCCAGGTCGCTGTTCAGATTGAATGGGAAATTGAAAAGGAACGCTC
CAAACGCTGGAATCCTGAAGTGCAGTTTACTTCAAACTATGGGAACCAGT
CTTCTATGTTGTGGGCTCCTGATACAACTGGGAAGTATACAGAGCCGCGG
GTTATTGGCTCTCGTTATTTGACTAATCATTTGTAAA

What is claimed is:

1. A viral vector comprising an adeno-associated virus (AAV) vector comprising an antigenic region of a coronavirus.

2. The viral vector of claim 1, wherein the AAV vector is naturally occurring primate AAV.

3. The viral vector of claim 1, wherein the AAV is an engineered or synthetic AAV.

4. The viral vector of claim 1, wherein the AAV vector is selected from AAV1, AAV4, AAV5, AAV6, AAV8, AAV11 and rh32.33.

5. The viral vector of claim 1, wherein the AAV vector is Rh32.33.

6. The viral vector of claim 1, wherein the AAV vector is AAV11.

7. The viral vector of any of the preceding claims, wherein the coronavirus is SARS-nCoV-2019.

8. The viral vector of any of the preceding claims, wherein the antigenic region of a coronavirus comprises one or more SPIKE regions or a portion thereof.

9. The viral vector of any of the preceding claims, wherein the SPIKE region or a portion thereof comprises an S1 domain or a RBD domain.

10. The viral vector of any of the preceding claims, wherein the SPIKE region or a portion thereof is stabilized.

11. The viral vector of claim 10, wherein the stabilization comprises mutagenesis or codon optimization, cross-linking, or heteromerization or homomerization.

12. The viral vector of claim 10, wherein the stabilization comprises removal of a furin cleavage site.

13. The viral vector of claim 10, wherein the stabilization comprises the addition of a trimerization C-terminal domain.

14. The viral vector of any of the preceding claims, wherein the viral vector is configured for intramuscular delivery.

15. The viral vector of any of the preceding claims, further comprising an adjuvant.

16. The viral vector of claim 15, wherein the adjuvant is selected from IL-2, IL-12, IL-18, IFN-gamma, or Niv G.

17. The viral vector of claim 15, wherein the viral vector comprises a nucleic acid encoding the adjuvant.

18. The viral vector of claim 15, wherein the adjuvant is Freund’s adjuvant or montanide.

19. The viral vector of any of the preceding claims, wherein the viral vector further comprises a nucleic acid sequence encoding kanamycin resistance.

20. A method of vaccinating a subject against coronavirus, the method comprising:
providing a viral vector comprising an adeno-associated virus (AAV) vector comprising an antigenic region of a coronavirus; and
delivering the viral vector to a subject.

21. The method of claim 20, wherein the subject is a human, a companion animal, an exotic animal, or a livestock animal.

22. The method of claim 20 or 21, wherein the viral vector is delivered intramuscularly.

23. The method of claim 20 or 21, wherein the viral vector is delivered intranasally or subcutaneously.

24. The method of any of claims 20-23, wherein the viral vector is delivered prior to exposure or infection.

25. The method of any of claims 20-23, wherein the viral vector is delivered following exposure or infection.

26. The method of any of claims 20-25, wherein the subject exhibits a protective immune response.

27. The method of claim 26, wherein the protective immune response comprises an increase in Th1 cells.

28. The method of claim 26, wherein the protective immune response comprises an increase in Treg cell ratios.

29. The method of claim 26, wherein the protective immune response comprises an amelioration of cytokine storms, ARDS and/or myocardial damage severity.

30. The method of any of claims 20-29, wherein the subject exhibits decreased lymphocyte counts, decreased erythrocyte sedimentation rates following delivery, and/or decreased C-reactive protein levels.

31. A method of producing the viral vaccine of claim 1, the method comprising:
providing a population of adherent or suspension cells;
infecting the adherent cells with the viral vector; and
culturing the infected cells under conditions in which the virus replicates.

32. The method of claim 31, wherein the cells are baculovirus cells.

33. The method of claim 31, wherein the culturing step is performed in a bioreactor.

34. A viral vector comprising an amino acid sequence having at least 95% sequence identity to SEQ ID NOs: 5, 10, 12, 14, 22, 24, 26, or 28.

35. The viral vector of claim 34, wherein the viral vector has an amino acid sequence having at least 99% sequence identity to SEQ ID NOs: 5, 10, 12, 14, 22, 24, 26, or 28.

36. The viral vector of claim 34, wherein the viral vector has an amino acid sequence having the sequence shown in SEQ ID NOs: 5, 10, 12, 14, 22, 24, 26, or 28.

37. A viral vector comprising a nucleic acid sequence having at least 95% sequence identity to SEQ ID NOs: 1, 2, 3, 4, 9, 11, 13, 15, 16, 17, 18, 23, 25, 27, or 29.

38. The viral vector of claim 37, wherein the viral vector has a nucleic acid sequence having at least 99% sequence identity to SEQ ID NOs: 1, 2, 3, 4, 9, 11, 13, 15, 16, 17, 18, 23, 25, 27, or 29.

39. The viral vector of claim 37, wherein the viral vector has a nucleic acid sequence having the sequence shown in SEQ ID NOs: 1, 2, 3, 4, 9, 11, 13, 15, 16, 17, 18, 23, 25, 27, or 29.


SEQUENCE LISTING (29 sequences)

SEQ ID NO: 1 - DNA, 4080 bp, Artificial
  Synthetic construct
ggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggatgcaatgaagagagggctctgctgtgtgctgctgctgtgtggagcagtcttcgtttcggccagccagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaa

SEQ ID NO: 2 - DNA, 2400 bp, Artificial
  Synthetic construct
ctgcgcgctcgctcgctcactgaggccgcccgggcaaagcccgggcgtcgggcgacctttggtcgcccggcctcagtgagcgagcgagcgcgcagagagggagtggttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggatgcaatgaagagagggctctgctgtgtgctgctgctgtgtggagcagtcttcgtttcggccagcgtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttctaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgc

SEQ ID NO: 3 - DNA, 2500 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggatgcaatgaagagagggctctgctgtgtgctgctgctgtgtggagcagtcttcgtttcggccagcgtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttctaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaactta

SEQ ID NO: 4 - DNA, 4810 bp, Artificial
  Synthetic construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagcggccgcctaagctctagcgtttaaacgggccctctagatgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaaatcggatcttaagatacattgatgagtttggacaaaccacaactagaatgcagtgaaaaaaatgctttatttgtgaaatttgtgatgctattgctttatttgtaaccattataagctgcaataaacaagttctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttac

SEQ ID NO: 5 - protein, 1273 aa, Artificial
  Synthetic protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 32 aa, Artificial
  Synthetic polypeptide
IKRMKQIEDKIEEIESKQKKIENEIARIKKIK

SEQ ID NO: 7 - protein, 29 aa, Artificial
  Synthetic polypeptide
GSGYIPEAPRDGQAYVRKDGEWVLLSTFL

SEQ ID NO: 8 - protein, 43 aa, Artificial
  Synthetic polypeptide
GGTGGNGTGRMKQIEDKIENITSKIYNITNEIARIKKLIGNRT

SEQ ID NO: 9 - DNA, 7488 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagcggccgcctagatgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggaagcgccagtagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccaccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcactacacctaaatcggatcttaagatacattgatgagtttggacaaaccacaactagaatgcagtgaaaaaaatgctttatttgtgaaatttgtgatgctattgctttatttgtaaccattataagctgcaataaacaagttctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 10 - protein, 1273 aa, Artificial
  Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - DNA, 5100 bp, Artificial
  Synthetic construct
ctgcgcgctcgctcgctcactgaggccgcccgggcaaagcccgggcgtcgggcgacctttggtcgcccggcctcagtgagcgagcgagcgcgcagagagggagtggttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggatgcaatgaagagagggctctgctgtgtgctgctgctgtgtggagcagtcttcgtttcggccagcgtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttctaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagggatctgggccactccctct

SEQ ID NO: 12 - protein, 245 aa, Artificial
  Synthetic polypeptide
MDAMKRGLCCVLLLCGAVFVSASVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 13 - DNA, 6832 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggatgcaatgaagagagggctctgctgtgtgctgctgctgtgtggagcagtcttcgtttcggccagccagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 14 - protein, 695 aa, Artificial
  Synthetic polypeptide
MDAMKRGLCCVLLLCGAVFVSASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 15 - DNA, 7353 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcggccgcctagatgccaccatgttcgtcttcctggtcctgctgcctctggtctcctcacagtgcgtcaatttcacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgccaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcggcctgcctcagggcttcagcgccctggagcccctggtggatctgcctatcggcatcaacatcacccggtttcagacactgctggccctgcacataagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaacatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggggtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggaagcgccagtagcgtggcctcccagtctatcatcgcctataccatgtccctgggcgtcgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccaccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcactacacctaaatcggatctaataaaatatctttattttcattacatctgtgtgttggttttttgtgtgctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 16 - DNA, 7353 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcggccgcctagatgccaccatgttcgtcttcctggtcctgctgcctctggtctcctcacagtgcgtcaatttcacaaatcggactcagctgccaagtgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaattatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagtgagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcacgatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggggtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaactctcccggaagcgccagtagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccaccggaggcagaggtgcagatcgaccggctgatcaccggccggctccagagcctccagacctatgtgacacagcagctgatcagggccgccgagatcagggccagcgccaatctggcagcaatcaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccttcgtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcactacacctaaatcggatctaataaaatatctttattttcattacatctgtgtgttggttttttgtgtgctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 17 - DNA, 7471 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatacagctccaccgcacatgccaccctccggatatattcgtctcgagcaaatcacttggcagtctagcggcggccgcctagatgccaccatgttcgtcttcctggtcctgctgcctctggtctcctcacagtgcgtcaatttcacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgccaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcggcctgcctcagggcttcagcgccctggagcccctggtggatctgcctatcggcatcaacatcacccggtttcagacactgctggccctgcacataagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaacatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggggtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggaagcgccagtagcgtggcctcccagtctatcatcgcctataccatgtccctgggcgtcgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccaccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcactacacctaaatcggatctaataaaatatctttattttcattacatctgtgtgttggttttttgtgtgctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 18 - DNA, 7471 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatacagctccaccgcacatgccaccctccggatatattcgtctcgagcaaatcacttggcagtctagcggcggccgcctagatgccaccatgttcgtcttcctggtcctgctgcctctggtctcctcacagtgcgtcaatttcacaaatcggactcagctgccaagtgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaattatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagtgagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcacgatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggggtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaactctcccggaagcgccagtagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccaccggaggcagaggtgcagatcgaccggctgatcaccggccggctccagagcctccagacctatgtgacacagcagctgatcagggccgccgagatcagggccagcgccaatctggcagcaatcaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccttcgtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcactacacctaaatcggatctaataaaatatctttattttcattacatctgtgtgttggttttttgtgtgctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 19 - DNA, 15 bp, Artificial
  Synthetic oligonucleotide
gtgcagccaaccgag

SEQ ID NO: 20 - DNA, 16 bp, Artificial
  Synthetic oligonucleotide
acacctcgccaaatgg

SEQ ID NO: 21 - DNA, 16 bp, Artificial
  Synthetic oligonucleotide
tctatcgtgcgctttc

SEQ ID NO: 22 - protein, 1273 aa, Artificial; identical to SEQ ID NO: 5
  Synthetic polypeptide

SEQ ID NO: 23 - DNA, 7488 bp, Artificial
  Synthetic construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagcggccgcctagatgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccggaagcgccagtagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacccaccggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcactacacctaaatcggatcttaagatacattgatgagtttggacaaaccacaactagaatgcagtgaaaaaaatgctttatttgtgaaatttgtgatgctattgctttatttgtaaccattataagctgcaataaacaagttctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 24 - protein, 1273 aa, Artificial; identical to SEQ ID NO: 10
  Synthetic polypeptide

SEQ ID NO: 25 - DNA, 5100 bp, Artificial; identical to SEQ ID NO: 11
  Synthetic polypeptide

SEQ ID NO: 26 - protein, 245 aa, Artificial; identical to SEQ ID NO: 12
  Synthetic polypeptide

SEQ ID NO: 27 - DNA, 6832 bp, Artificial; identical to SEQ ID NO: 13
  Synthetic construct

SEQ ID NO: 28 - protein, 695 aa, Artificial; identical to SEQ ID NO: 14
  Synthetic polypeptide

SEQ ID NO: 29 - DNA, 7437 bp, Artificial
  Synthetic construct
ctagtttgcttgttaatcaataaaccgtttaattcgtttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttcgcgcgcttggcgtaatcatggtcatagctgtttcctgtgtgaaattccacagcctggggtgcctaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagaaataataaaaaagccggattaataatctggctttttatattctctctctagtatataaacgcagaaaggcccacccgaaggtgagccagtgtgactctagtattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgagacggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagctctagtaaaataataaaaaagccggattaataatctggctttttatattctctctctagtatataaacgcagaaaggcccacccgaaggtgagccagtgtgacggcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcggcgaattgggtaccgggccccccctcgaggtcgacggtatcgtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaaa